(19) United States
(12) Patent Application Publication
(10) Pub. No.: US 2024/0053339 A1
(43) Pub. Date: Feb. 15, 2024

(54) HIGH THROUGHPUT METHODS AND PRODUCTS FOR SARS-COV-2 SERO-NEUTRALIZATION ASSAY

(71) Applicants: THERAVECTYS, Paris (FR); INSTITUT PASTEUR, Paris Cedex 15 (FR)

(72) Inventors: Pierre CHARNEAU, Paris (FR); François ANNA, Paris (FR); Fabien NEVO, Paris (FR); Amandine NOIRAT, Paris (FR); Yves JACOB, Paris (FR); Phillipe SOUQUE, (US)

(21) Appl. No.: 18/245,987

(22) PCT Filed: Sep. 21, 2021
(86) PCT No.: PCT/EP2021/075970

§ 371 Date: Mar. 20, 2023

Related U.S. Application Data
(60) Provisional application No. 63/107,896, filed on Oct. 30, 2020.
(60) Provisional application No. 63/080,939, filed on Sep. 21, 2020.

(51) Int. Cl. G01N 33/569; C12N 15/86; C07K 14/165; C07K 16/10; C12N 9/12; G01N 33/58

(52) U.S. Cl. CPC G01N 33/56983; C12N 15/86; C07K 14/165; C07K 16/1003; C12N 9/12; G01N 33/58; C12Y 207/07; G01N 2333/165; G01N 2333/155; G01N 2469/20; C12N 2740/16043; C12N 2740/16022

(57) ABSTRACT

Pseudotyped lentiviral vector particles bearing a SARS-CoV-2 S protein and comprising a heterologous polynucleotide that encodes a label or a recombinase. Compositions or kits comprising the pseudotyped lentiviral vector particles and a mammalian cell expressing an Angiotensin-converting Enzyme 2 (ACE2) protein. Methods of assaying for the presence of neutralizing antibodies against a SARS-CoV-2 S protein in a sample comprising antibodies by providing pseudotyped lentiviral vector particles bearing a SARS-CoV-2 S protein and comprising a heterologous polynucleotide that encodes a label or a recombinase; providing mammalian cells expressing an ACE2 protein; contacting the mammalian cells expressing the ACE2 protein with the pseudotyped lentiviral vector particles bearing a SARS-CoV-2 S protein in the presence of a sample comprising antibodies; and assaying for the presence of a label in the mammalian cells.

### FIELD OF THE INVENTION

This disclosure relates generally to the fields of cells, kits, and methods relating to SARS-CoV-2.

### BACKGROUND OF THE INVENTION

About four months after the initial description of atypical pneumonia cases in Wuhan in December 2019, COVID-19 has become a major pandemic threat. As of Apr. 14, 2020, about half of the human population is under confinement, 2 million infections have been officially diagnosed, with 121,000 fatalities and 0.5 million recovered cases. COVID-19 is caused by SARS-CoV-2^{1,2}, a betacoronavirus displaying 80% nucleotide homology with Severe Acute Respiratory Syndrome virus (now termed SARS-CoV-1), that was responsible for an outbreak of 8,000 estimated cases in 2003.

PCR-based tests are widely used for COVID-19 diagnosis and for detection and quantification of SARS-CoV2 RNA^{3,4,5}. These virological assays are instrumental to monitor individuals with active infections. The average virus RNA load is 10⁵ copies per nasal or oropharyngeal swab at day 5 post symptom onsets and may reach 10⁸ copies⁶. A decline occurs after days 10-11, but viral RNA can be detected up to day 28 post-onset in recovered patients at a time when antibodies (Abs) are most often readily detectable^{6,7}. Disease severity correlates with viral loads, and elderly patients, who are particularly sensitive to infection, display higher viral loads^{6,7}.

Serological assays are also being implemented. Anti-Spike (S) and Nucleoprotein (N) humoral responses in COVID-19 patients are assessed, because the two proteins are highly immunogenic. The viral spike (S) protein allows viral binding and entry into target cells. S binding to a cellular receptor, angiotensin-converting enzyme 2 (ACE2) for SARS-CoV-1 and -CoV2, is followed by S cleavage and priming by the cellular protease TMPRSS2 or other endosomal proteases⁸. S genes from SARS-CoV and -CoV2 share 76% amino-acid similarity². One noticeable difference between the two viruses is the presence of a furin cleavage site in SARS-CoV2, which is suspected to enhance viral infectivity². The structures of S from SARS-CoV-1 and Co-V-2 in complex with ACE2 have been elucidated⁹⁻¹¹. S consists of three S1-S2 dimers, displaying different conformational changes upon virus entry leading to fusion^{9,10,12}. Some anti-S antibodies, including those targeting the receptor binding domain (RBD), display a neutralizing activity, but their relative frequency among the generated anti-SARS-CoV-2 antibodies during infection remains poorly characterized. The nucleoprotein N is highly conserved between SARS-CoV1 and -CoV2 (96% amino-acid homology). N plays a crucial role in subgenomic viral RNA transcription and viral replication and assembly.

Serological assays are currently being performed using in-house, pre-commercial versions or commercially available ELISA-based diagnostics tests^{6,7,13-15}. Other techniques, including point-of-care and auto-tests are also becoming available. In hospitalized patients, seroconversion is typically detected between 5-14 days post symptom onset, with a median time of 5-12 days for anti-S IgM and 14 days for IgG and IgA^{6,7,13-16}. The kinetics of anti-N response was described to be similar to that of anti-S, although N responses might appear earlier¹⁵⁻¹⁷. Anti-SARS-CoV-2 antibody titers correlate with disease severity, likely reflecting higher viral replication rates and/or immune activation in patients with severe outcome. Besides N and S, antibody responses to other viral proteins (mainly ORF9b and NSP5) were also identified by antibody microarray¹⁷.

Neutralization titers observed in individuals infected with other coronaviruses, such as MERS-CoV, are considered to be relatively low^{6,18}. With SARS-CoV-2, neutralizing antibodies (Nabs) have been detected in symptomatic individuals^{6,8,19,20}, and their potency seems to be associated with high levels of antibodies. Neutralization is assessed using plaque neutralization assays, microneutralization assays, or inhibition of infection with viral pseudotypes carrying the S protein^{6,8,19-21}. Of note, potent monoclonal NAbs that target RBD have been cloned from infected individuals²². Whether asymptomatic infections, which are currently often undocumented²³, and most likely represent the majority of SARS-CoV-2 cases, lead to protective immunity, and whether this immunity is mediated by NAbs, remain outstanding questions.

A need exists for antibody-based assays for SARS-CoV-2 infection, including seroneutralization assays. This disclosure meets these and other needs.

### SUMMARY OF THE INVENTION

It is of paramount importance to evaluate the prevalence of both asymptomatic and symptomatic cases of SARS-CoV-2 infection and to characterize their antibody response profile, including characterizing seroneutralization. To this end the inventors have developed methods and compositions useful to characterize seroneutralization of patients who are or have been infected with SARS-CoV-2.

Accordingly, in a first aspect this disclosure provides pseudotyped lentiviral vector particles bearing a SARS-CoV-2 S protein. In some embodiments the SARS-CoV-2 S protein has an amino acid sequence at least 95% identical to SEQ ID NO: 1. In some embodiments the SARS-CoV-2 S protein is expressed from a coding sequence having a nucleotide sequence at least 80% identical to SEQ ID NO: 2. In some embodiments the pseudotyped lentiviral vector particles further comprise a heterologous polynucleotide that encodes a label. In some embodiments the label is a fluorescent protein, such as green fluorescent protein. In some embodiments the label is an enzyme, such as luciferase or nano-luc.

In a second aspect this disclosure provides a composition or a kit comprising a pseudotyped lentiviral vector particle bearing a SARS-CoV-2 S protein, and a mammalian cell expressing an Angiotensin-converting Enzyme 2 (ACE2) protein. In some embodiments the SARS-CoV-2 S protein has an amino acid sequence at least 95% identical to SEQ ID NO: 1. In some embodiments the SARS-CoV-2 S protein is expressed from a coding sequence having a nucleotide sequence at least 80% identical to SEQ ID NO: 2. In some embodiments the pseudotyped lentiviral vector particles further comprise a heterologous polynucleotide that encodes a label. In some embodiments the label is a fluorescent protein, such as green fluorescent protein. In some embodiments the label is an enzyme, such as luciferase or nano-luc. In some embodiments the mammalian cell further expresses the serine protease TMPRSS2. In some embodiments the cell is a human cell. In some embodiments the human cell is a 293T cell or a HeLa cell. In some embodiments the ACE2 protein has an amino acid sequence at least 95% identical to SEQ ID NO: 3. In some embodiments the ACE2 protein is expressed from a coding sequence having a nucleotide sequence at least 80% identical to SEQ ID NO: 4. In some embodiments the composition or the kit further comprises a human serum. In some embodiments the composition or the kit further comprises a SARS-CoV-2 S protein binding agent. In some embodiments the composition or the kit further comprises an ACE2 binding agent. In some embodiments the composition or the kit further comprises reagents for visualizing the label.

In a third aspect this disclosure provides the use of a lentivector particle according to the first aspect and/or a composition, kit or system according to the second aspect, to detect the presence of neutralizing antibodies against SARS-CoV-2 in a sample comprising antibodies.

In a fourth aspect this disclosure provides a lentivector particle according to the first aspect and/or a composition, kit or system according to the second aspect for use in detecting the presence of neutralizing antibodies against SARS-CoV-2 in a sample comprising antibodies.

In a fifth aspect, this disclosure provides methods of assaying for the presence of neutralizing antibodies against a SARS-CoV-2 S protein in a sample comprising antibodies. The methods may comprise a) providing pseudotyped lentiviral vector particles bearing a SARS-CoV-2 S protein and comprising a heterologous polynucleotide that encodes a label; b) providing mammalian cells expressing an ACE2 protein; c) contacting the pseudotyped lentiviral vector particles bearing a SARS-CoV-2 S protein with a sample comprising antibodies; d) contacting the mammalian cells expressing an ACE2 protein with the pseudotyped lentiviral vector particles bearing a SARS-CoV-2 S protein; and e) assaying for the presence of the label in the mammalian cells. In some embodiments, c) and d) occur sequentially. The methods may comprise a) providing pseudotyped lentiviral vector particles bearing a SARS-CoV-2 S protein and comprising a heterologous polynucleotide that encodes a recombinase, and in particular a Cre recombinase, more particularly a Cre recombinase comprising or consisting of an amino acid sequence that is at least 60%, 70%, 80%, 85%, 90%, 91%, 92%, 93%, 94%, 95%, 96%, 97%, 98%, or 99% identical to SEQ ID NO: 14; b) providing mammalian cells expressing an ACE2 protein and comprising a nanolox nucleotide sequence comprising or consisting of a sequence that is at least 90%, 91%, 92%, 93%, 94%, 95%, 96%, 97%, 98%, or 99% identical to SEQ ID NO: 11; c) contacting the pseudotyped lentiviral vector particles bearing a SARS-CoV-2 S protein with a sample comprising antibodies; contacting the mammalian cells expressing an ACE2 protein with the pseudotyped lentiviral vector particles bearing a SARS-CoV-2 S protein and comprising the heterologous polynucleotide that encodes the recombinase; and e) assaying for the presence of the expression of the Nanoluc protein encoded in the Nanolox sequence in the mammalian cells. In some embodiments, c) and d) occur simultaneously. In some embodiments, c) comprises incubating the pseudotyped lentiviral vector particles bearing a SARS-CoV-2 S protein with the sample comprising antibodies for at least 15 minutes prior to performing d). In some embodiments, c) comprises incubating the pseudotyped lentiviral vector particles bearing a SARS-CoV-2 S protein with the sample comprising antibodies for from 30 to 60 minutes prior to performing d). In some embodiments, d) comprises incubating the mammalian cells expressing an ACE2 protein with the pseudotyped lentiviral vector particles bearing a SARS-CoV-2 S protein for from 48 to 72 hours. In some embodiments, the mammalian cells in d) are adhered to a solid support. In some embodiments, the mammalian cells in d) are in a suspension culture. In some embodiments, the SARS-CoV-2 S protein has an amino acid sequence at least 95% identical to SEQ ID NO: 1. In some embodiments, the SARS-CoV-2 S protein is expressed from a coding sequence having a nucleotide sequence at least 80% identical to SEQ ID NO: 2. In some embodiments, the label is a fluorescent protein, such as green fluorescent protein. In some embodiments, the label is an enzyme, such as luciferase or nano-luc. In some embodiments, the mammalian cells further express the serine protease TMPRSS2. In some embodiments, the cell is a human cell. In some embodiments, the human cell is a 293T cell or a HeLa cell. In some embodiments, the ACE2 protein has an amino acid sequence at least 95% identical to SEQ ID NO: 3. In some embodiments, the ACE2 protein is expressed from a coding sequence having a nucleotide sequence at least 80% identical to SEQ ID NO: 4. In some embodiments, the sample is a human serum. In some embodiments, assaying for the presence of the label in the mammalian cells comprises measuring the level of the label in the mammalian cells. In some embodiments, the level of the label is less than or equal to a pre-determined threshold or a measured control value, indicating the presence of neutralizing antibodies against a SARS-CoV-2 S protein in the sample. In some embodiments, the level of the label is equal to or greater than a pre-determined threshold or a measured control value, indicating the absence of neutralizing antibodies against a SARS-CoV-2 S protein in the sample.

### BRIEF DESCRIPTION OF THE DRAWINGS

FIG. 1. Generation of Pseudotyped Lentiviral Vector Particles Harboring SARS-CoV-2 Spike and Coding for Luciferase or GFP Reporter Genes. An embodiment of making pseudotyped lentiviral vector particles harboring SARS-CoV-2 spike and coding for luciferase or GFP reporter genes is shown. Lentiviral vector particles harboring the CV2 spike are replication deficient and can be handled in BSL2 cell culture facilities.

FIG. 2. Method of Detection and Quantitation of Neutralizing Antibodies specific to SARS-2 CoV2. An embodiment of a method of detection and quantitation of neutralizing antibodies specific to SARS-2 CoV2 is shown. This simple sero-neutralization assay can be rapidly provided for millions of tests. It can be automated for high throughput analysis of patient's sera to predict their immune protection.

FIG. 3. Generation of a Stable 293T-hACE2 Cell Line. Transduction efficiency of unmodified 293T (293T WT) cells or stable hACE-2 expressing 293T cells (293T hACE2) with a lentiviral vector pseutodyptes with either SARS-CoV-2 spike envelope protein (S) or Vesicular Stomatitis Virus Glycoprotein (VSF-G). 2×10⁴ cells were transduced with 5 μl of native vector production supernatant (black bars) or heat-inactivated at 72° C. for 10 minutes (white bars). Luciferase expression was monitored 48 h after transduction. (Columns represent mean of two experiments.)

FIG. 4. Analysis of the Transduction Capacity of Different Lentiviral Pseudo-Particles Pseudotyped With SARS-CoV-2 Protein S in Model Line 293T-hACE2. Viral particles pseudotyped with the SARS-CoV-2 protein S. ILV luc: Second generation lentiviral vector expressing Firefly luciferase. rMLV: Recombinant Moloney Virus. rHIV: Recombinant HIV Virus. NC: lentiviral vector ILV luc not concentrated. UF: Lentiviral vector ILV luc concentrated by ultra-filtration (1/100th).

Controls (Pseudotyped with the amphotropic envelope VSV-G). GFP VSV-G: Second generation lentiviral vector expressing GFP. Luc VSV-G: Second generation lentiviral vector.

These data indicate that the lentiviral vector is superior to the retroviral recombinant vectors; that the quantity of envelope plasmid has little effect on the capacity of vector transduction; and that ultrafiltration is not possible for this type of envelope.

FIG. 5. Comparison of the permissiveness of different lineages over-expressing the hACE2 receptor to lentiviral particles pseudotyped with the protein S of SARS-CoV-2. The cells potentially permissive to infection by SARS-CoV-2 (Vero, A549, and Caco2) have been transduced with an integrative lentiviral vector over-expressing the hACE2 receptor. The permissiveness of these cells to transduction by different lentiviral vectors pseudotyped with the S protein of SARS-CoV-2 was then evaluated: PS-102/5 and AN85 express the firefly luciferase under the control of a CMV promoter while the AN87 vector expressed it under the control of a UBC promoter. A vector pseudotyped with the VSV-G envelope serves as a control. Conclusion: The 293T-hACE2 lineage remains as the best model of transduction by a vector pseudotyped by the S protein.

FIG. 6. Development of a HELA-hACE2 Cell Line and Validation For Seroneutralization Assays. Results are comparable with the 293T lineage with the same resolution of 3 logs between a negative serum and a fully neutralizing positive serum. An advantage of the HELA lineage is the ease of its use in a 384 well plate format compared to 293T cells

FIG. 7. Analysis of CORSER Cohort and Correlation With Others Tests.

FIG. 8. Analysis of the capacity of seroneutralisation of seras of the CORSER cohort and determination of the FRNT 50. Experiment using 20,000 293-hACE2 cells with 0.5 μl of the CMV-Luc (Firefly) vector pseudotyped Spike SARS-CoV-2. Reading of the luminescense at 48 hours.

FIG. 9. Analysis of Bichat-1 and Bichat-2 Cohorts.

FIG. 10. HEK293T-hACE2-GFP cell line generation. (A) Screening of clones for constitutive GFP expression and Firefly expression after transduction with S pseudotyped Lentiviral vector. Arrow indicates selected clone (n° 15). (B) Correlation between GFP fluorescence and number of cell per well (C) Viability assay: Distribution of GFP fluorescence in 30 wells plated with 30 000 cells after 72 h of culture at 37° C. 5% CO2. 2 treated wells (8 μM of aphidicolin preventing cell division) are indicated as control.

FIG. 11. Complementary Analysis of Bichat Cohort.

FIGS. 12A and 12B. (A) Example of FRNT50 calculation of human sera and evaluation of cross-reactivity of anti-S Sars-Cov-1 polyclonal antibodies. (B) Validation of the effect of hydroxychloroquine on the capacity of fusion of viral particles pseudotyped with the S Sars-Cov-2 protein.

FIGS. 13A and 13B. (A) Schematic Workflow of Two-Step Revelation protocol. (B) Schematic Workflow of One-Step Revelation protocol.

FIG. 14. Comparison between Pseudovirus neutralization assay and Microneutralisation (MNT) assay.

FIG. 15. Pseudovirus neutralization assay. (A) Correlation between Pseudovirus neutralization assay and other serologicals assays. (B) Correlation between symptom onset and % of Neutralization illustrating humoral response maturation.

FIG. 16. Serological responses to SARS-CoV-2 among Institut Curie workers using PNT assay. Sera from prepandemic samples from a blood bank, prepandemic patients (Breast Cancer), COVID-19 patients (RT-PCR positive) and Institut Curie Workers were evaluated using a pseudoneutralization (PNT) assay of this disclosure. For PNT assay, values after ID₅₀ calculation are represented (See FIGS. 20 and 21 for calculation details and 17 for raw values). Negative sera are represented with an ID₅₀ below detection limit (40).

FIG. 17. PNT raw data for cohorts analysis. Serum dilution factor: 1/40. Positive samples are below the threshold level. Threshold is calculated on raw values of prepandemic sera from a blood bank (see FIGS. 20 and 21). ID₅₀ was determined on positive samples (see FIGS. 16 and 21).

FIG. 18. Temporal distribution of symptoms appearance and serology correlates with COVID-19 outbreak in France. Temporal distribution of serological test result according to first symptom onset. Individual test results for the PNT assay are plotted.

FIG. 19A-19C. Serological profile follow-up overtime. Serum from seropositive workers for pseudo-neutralization activity (A-C) at the first blood sampling (t₀) were reassessed 6-8 weeks later (t₁). A: Test values according to delay between symptom reporting and the two sera analysis: t₀ (blue dots) and t₁ (red dots). Linear regression is plotted. Coefficient of determination and associated p value are indicated. B: Whisker-plots summarizing test value for both tests (t₀ and T1). Statistical significance was determined using a Wilcoxon test (****:p<0.0001). C: individual follow-up of seropositive workers with a decreasing value. Variation of mean (t₀/t₁) is indicated in %.

FIG. 20A-20B. (A) Control of hACE2 expression in 293T::hACE2 reporter cell line by FACS. To verify the expression and membrane location of hACE2 protein, parental cell line (293T WT) and hACE2 expressing clone (293T::hACE2) were first labelled polyclonal antibodies anti-hACE2 (AF933, R&D) then a PE-coupled secondary antibody (A32849, Invitrogen). Samples were acquired on a Attune FACS (ThermoFisher). (B) Control of 293T::hACE2 permissivity et specificity to S-pseudotyped lentiviral vector transduction. Parental cell line and hACE2 clone were transduced with S-pseudotyped lentiviral vector (or VSV-G, an amphotropic envelope as control). To assess that signal is specific to vector particles, equivalent heat-inactivated samples (70° C. 30 min in a water bath) were also tested.

FIG. 21A-21C. (A) Threshold determination on different serum collections. To setup up the experimentation, Min-max values are determined on untransduced cells and prepandemic serum (dilution 1/40) respectively, Covid-19 patients are used as positive controls. To define positiveness threshold with a confidence index >99%, this value is set at Mean (prepandemic)−3 Standard deviation. During sample analysis, all samples are firstly evaluated for positiveness at dilution 1/40. If the value is below threshold, then ID50 is determined as described below in a second experiment. (B-C) Dilution curves and ID50 determination. First, raw datas (grey points) (B) are transformed into percentage of neutralization (C). This percentage is determined according to mean of prepandemic serums (0%) and Untransduced cells (100%) values. Second, a non-linear regression is performed (plotted line in graph G) to determine the theorical dilution that give a 50% inhibition (ID50). A prepandemic sample (Black point) is shown as negative example. Detection limit was set at 40 considering the maximum reached value by prepandemic samples.

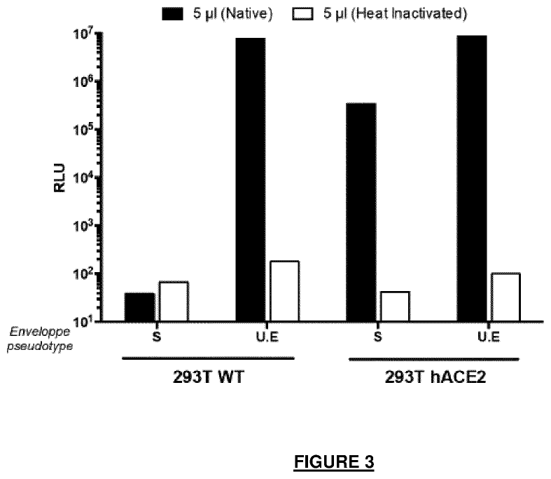

FIG. 22A to 22I: Correlation of neutralizing activity of serum and mucosal antibodies to SARS-CoV-2.
A. Example of delay between symptom onset and neutralizing activity. Neutralizing activity of 52 sera (dilution 1:40) from 38 SARS-CoV-2 infected patients was determined by pseudovirus neutralisation assay. Orange curve represents significant sigmoidal interpolation (p=0.0082). Grey dotted lines represent 95% confidence intervals curves.
B. Inhibitory dilution curves of 18 sera measured by pseudovirus neutralization assay at different indicated dilutions of serum. Samples used for this analysis were collected between day 6 (Light Blue) and 24 (Dark Blue) after symptoms onset.
C. Neutralizing activity of purified IgG was measured at indicated concentrations from 18 sera collected between day 6 and day 24 post-symptoms onset. Curves were drawn according to non-linear regression. Color code as above.
D. Neutralizing activity of purified IgA from paired FIG. 3C samples, analysed as in FIG. 3C. Curves corresponding to samples endowed with low IgA neutralization potential are light blue.
E. Paired purified IgA and IgG IC50 values in samples tested in FIGS. 3C and D. P value was calculated using Wilcoxon test (* p<0.05).
F. Comparison of serum anti-RBD IgA (main panel) or IgG (insert) levels measured by photonic ring immunoassay with neutralizing capacity of corresponding purified isotypes measured by pseudovirus neutralization assay. Spearman coefficient (r) and p value (p) are indicated.
G. Neutralizing activity of bronchoalveolar lavages (BAL) collected in 10 SARS-CoV-2 patients between day 4 and 23 after symptoms onset (clinical characteristics are detailed in Table S5). Indicated BAL dilutions were tested using pseudovirus neutralization assay. Bronchoalveolar lavages obtained from SARS-CoV-2 negative patients (n=3) showed no neutralization activity (dotted grey lines). Each colored line represent one patient.
H. Neutralizing activity and anti-RBD IgA levels (both tested at dilution 1:4) of saliva collected in 10 SARS-CoV-2 patients between day 49 and 73 after symptoms onset. Spearman coefficient (r) and p value (p) are indicated.
I. Anti-RBD levels in paired saliva and serum from patients tested in FIG. 3H. P value was calculated using Wilcoxon test (** p<0.01).

FIG. 23: Correlation of neutralizing activity measured by Pseudotyped Neutralisation Assay and a real Neutralisation assay (S-Fuse). Example shows correlation for both serum, purified IgA and Purified IgG.

FIG. 24: Method of Detection and Quantitation of Neutralizing Antibodies specific to SARS CoV2. An embodiment of a method of detection and quantitation of neutralizing antibodies specific to SARS CoV2 is shown. This simple sero-neutralization assay can be rapidly provided for millions of tests. It can be automated for high throughput analysis of patient's sera to predict their immune protection.

FIG. 25: Cloning of the 293T::hACE2::Nanolox cell line. (A) Induction level of the Nanoluc expression by 293T cells transduced with different MOI of LV UBC-nanolox vector and then activated with a pseudotyped Spike vector expressing the CRE recombinase. (B-C) Cloning of the population identified in A and evaluation of the induction level by the pseudotyped Spike vector expressing the CRE recombinase for each clone. (B) absolute value with (Spike-Cre) or without (−) the vector. (C) fold variation of the ratio with vector (Spike-Cre)/without vector (−).

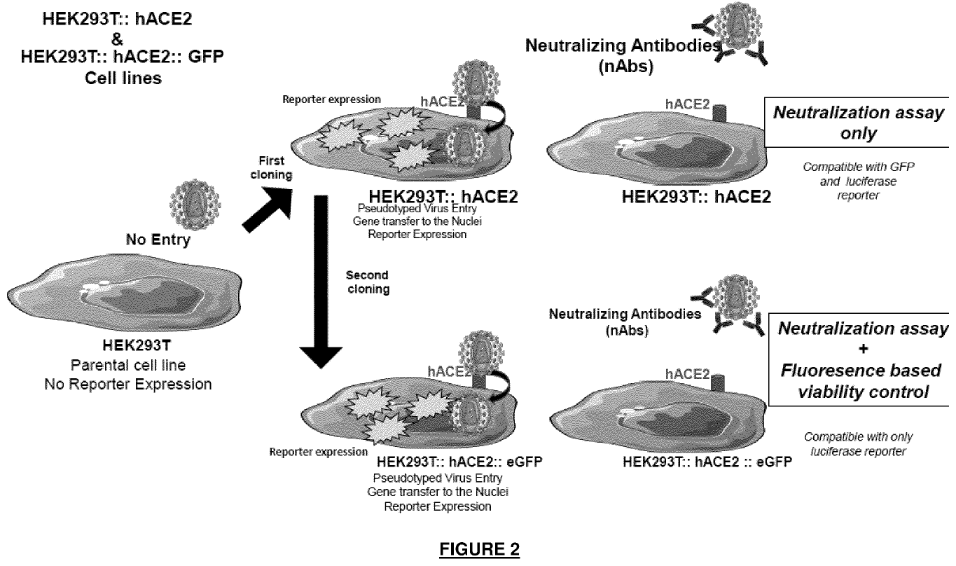

FIG. 26: Seroneutralization test using either a monoclonal antibody having a high affinity for Spike (high) or a monoclonal antibody having a low affinity for Spike (low) implementing either the system of FIG. 24 (Spike-Cre:Low-Cre or High-Cre) or the system of FIG. 2 (Spike-Luciferase:Low-Luc or High-Luc). (A) Comparison of the raw values of RLU/s between the two systems (either Cre-Nanolox (Cre) system or the Luciferase Firefly (Luc) system). (B) Comparison of the adjusted values of RLU/s between the two systems (either Cre-Nanolux (Cre) system or the Luciferase Firefly (Luc) system).

FIG. 27 is a Schematic representation of pTRIPΔU3.hUBC-hACE2.

FIG. 28 is a Schematic representation of pTRIPΔU3.CMV-LucF-WPREm.

FIG. 29 is a Schematic representation of pCMV.S High

### DETAILED DESCRIPTION OF THE INVENTION

It is of paramount importance to evaluate the prevalence of both asymptomatic and symptomatic cases of SARS-CoV-2 infection and their antibody response profile, including seroconversion. The examples describe a novel format of seroconversion test for SARS-CoV-2. In certain embodiments the test is based on the one hand on a lentiviral vector pseudotyped by the envelope of SARS-CoV-2 (protein S or Spike) and expressing a reporter gene, Luciferase or nanoLuc, and on the one hand of an optimized target cell line, stably expressing the ACE2 receptor. The rate of seroneutralization may be determined after incubation of the serum with the pseudotypes, transduction of the ACE2 target cells and reading of the luminescence. The S pseudotypes are non-replicative and can be handled in BSL2 confinement. The production of the pseudotypes and of the target cell line is not limiting and can be ensured up to several million tests per week. A simple protocol has been developed and adapted on a robotic platform, which can enable a work-flow on the order of 50 to 100,000 tests per week/robot.

### SARS-CoV-2 S Protein and Nucleic Acid

Various aspects of this disclosure incorporate a SARS-CoV-2 S Protein. In a preferred embodiment the SARS-CoV-2 S Protein comprises or consists of the following amino acid sequence (UniProtKB—P0DTC2 (SPIKE_SARS2); SEQ ID NO: 1):

MFVFLVLLPLVSSQCVNLITRIQLPPAYTNSFTRGVYYPD
KVFRSSVLHSTQDLFLPFFSNVTWFHAIHVSGINGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNERV
QPTESIVRFPNITNLCPFGEVENATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGENCYFPLQSYGFQPTNGVGYQPYRVVVLSFELLHA
PATVCGPKKSTNLVKNKCVNFNFNGLTGTGVLTESNKKEL
PFQQFGRDIADITDAVRDPQTLEILDITPCSFGGVSVITP
GINTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPINFTI
SVTTEILPVSMTKTSVDCTMYICGDSTECSNLLLQYGSFC
TQLNRALTGIAVEQDKNTQEVFAQVKQIYKTPPIKDFGGE
NFSQILPDPSKPSKRSFIEDLLENKVTLADAGFIKQYGDC
LGDIAARDLICAQKFNGLTVLPPLLTDEMIAQYTSALLAG
TITSGWTFGAGAALQIPFAMQMAYRENGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVIQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHI
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT

In some embodiments the SARS-CoV-2 S Protein comprises or consists of an amino acid sequence that is at least 90%, 91%, 92%, 93%, 94%, 95%, 96%, 97%, 98%, or 99% identical to SEQ ID NO: 1.

In some embodiments the SARS-CoV-2 S Protein comprises or consists of an amino acid sequence that has 1, 2, 3, 4, 5, 6, 7, 8, 9 or 10 amino acid changes relative to SEQ ID NO: 1. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In a preferred embodiment the SARS-CoV-2 S Protein is encoded by a nucleotide sequence that comprises or consists of the following nucleotide sequence (SEQ ID NO: 2):

ATGTTCGTGTTTCTGGTGCTGCTGCCACTGGTGTCCAGTC
AGTGCGTGAACCTGACCACACGAACACAGCTGCCACCAGC
CTACACCAATAGCTTCACCCGCGGAGTGTACTACCCCGAC
AAGGTGTTCCGCAGCAGCGTGCTGCATAGCACCCAGGATC
TGTTTCTGCCCTTCTTCAGCAACGTGACCTGGTTCCACGC
CATCCACGTGTCCGGCACCAATGGCACCAAGCGCTTCGAT
AATCCCGTGCTGCCCTTCAACGATGGCGTGTACTTTGCCA
GCACCGAGAAGTCCAATATCATCCGCGGCTGGATCTTCGG
CACCACACTGGATAGCAAGACCCAGAGCCTGCTGATCGTG
AACAACGCCACCAACGTGGTCATCAAAGTGTGCGAGTTCC
AGTTCTGCAACGACCCCTTCCTGGGCGTCTACTACCACAA
GAACAACAAGAGCTGGATGGAAAGCGAGTTCCGCGTGTAC
AGCAGCGCCAACAACTGCACCTTCGAGTACGTGTCCCAGC
CATTCCTGATGGACCTGGAAGGCAAGCAGGGCAACTTCAA
GAACCTGCGCGAGTTCGTGTTCAAGAACATCGACGGCTAC
TTCAAAATCTACAGCAAGCACACCCCAATCAACCTCGTGC
GCGATCTGCCACAGGGATTCAGTGCTCTGGAACCCCTGGT
GGATCTGCCCATCGGCATCAACATTACCCGCTTTCAGACA
CTGCTGGCCCTGCACCGCAGTTACTTGACACCAGGCGATA
GCAGCAGTGGATGGACAGCTGGTGCCGCCGCTTACTACGT
TGGATATCTGCAGCCACGCACCTTTCTGCTGAAGTACAAC
GAGAACGGCACCATCACCGACGCCGTGGATTGTGCTCTCG
ATCCCCTGAGCGAGACAAAGTGCACCCTGAAGTCCTTCAC
CGTCGAGAAGGGCATCTACCAGACCAGCAATTTCCGCGTG
CAGCCCACCGAGAGCATCGTGCGCTTCCCCAATATCACCA
ATCTGTGCCCCTTCGGCGAGGTGTTCAATGCCACACGCTT
TGCCTCCGTGTACGCCTGGAATCGCAAGCGCATTAGCAAC
TGCGTGGCCGACTACTCCGTGCTGTACAATAGCGCCAGCT
TCAGCACCTTCAAGTGCTACGGCGTGTCACCCACCAAGCT
GAACGACCTGTGCTTCACCAATGTGTACGCCGACAGCTTC
GTGATCCGCGGAGATGAAGTGCGACAGATTGCCCCAGGCC
AGACCGGCAAGATCGCCGACTACAATTACAAGCTGCCCGA
CGACTTCACCGGCTGCGTGATCGCCTGGAACAGCAACAAC
CTGGATTCCAAAGTCGGCGGCAACTACAACTACCTGTACC
GCCTGTTCCGCAAGAGCAATCTGAAGCCCTTCGAGCGCGA
CATCAGCACCGAAATCTACCAGGCCGGAAGCACCCCATGC
AACGGCGTGGAAGGCTTCAACTGCTACTTCCCACTGCAGT
CCTACGGATTTCAGCCCACAAATGGCGTGGGCTACCAGCC
ATATCGAGTGGTGGTGCTGAGCTTCGAACTGCTGCATGCT
CCAGCTACCGTGTGCGGCCCCAAGAAGAGTACCAACCTGG
TCAAGAACAAATGCGTGAACTTCAACTTCAACGGCCTGAC
CGGAACCGGCGTGCTGACCGAGAGTAACAAGAAGTTCCTG
CCATTCCAGCAGTTTGGCCGCGACATTGCCGATACAACCG
ATGCCGTTCGCGATCCCCAGACCTTGGAGATCCTGGATAT
TACCCCATGCTCCTTCGGCGGCGTGTCCGTGATTACACCA
GGCACCAATACCAGCAACCAGGTGGCCGTTCTGTACCAGG
ATGTGAATTGCACAGAGGTGCCCGTGGCCATTCACGCCGA
TCAATTGACACCAACATGGCGCGTGTACTCCACCGGCAGC
AATGTGTTTCAAACCCGCGCTGGATGCCTGATTGGAGCCG
AGCACGTGAACAATAGCTACGAGTGCGATATCCCCATCGG
AGCCGGAATCTGCGCCTCCTATCAGACCCAGACCAATAGT
CCACGACGAGCCCGAAGTGTGGCCAGCCAGAGCATCATTG
CCTATACCATGAGCCTGGGCGCCGAGAATAGCGTGGCCTA
CTCCAACAACAGCATTGCTATCCCCACCAACTTCACCATC
AGCGTGACCACCGAGATCCTGCCAGTGTCCATGACCAAGA
CCAGCGTGGACTGCACCATGTACATCTGCGGAGATAGCAC
CGAGTGCAGCAACCTGCTGCTGCAGTACGGAAGTTTCTGC
ACCCAGCTGAATCGCGCCCTGACAGGCATTGCCGTGGAAC
AGGATAAGAACACCCAAGAGGTGTTCGCCCAAGTGAAGCA
AATCTACAAGACCCCACCAATCAAGGATTTCGGCGGCTTC
AATTTCAGCCAGATTCTGCCCGATCCAAGCAAGCCCAGCA
AGCGCAGCTTCATCGAGGACCTGCTGTTCAACAAAGTGAC
ACTGGCCGACGCCGGATTCATCAAGCAGTATGGCGATTGC
CTGGGCGATATTGCCGCACGCGATCTGATTTGCGCCCAGA
AGTTTAACGGACTGACCGTCCTGCCACCACTGCTGACAGA
TGAGATGATCGCCCAGTACACAAGTGCCCTGCTGGCCGGA
ACCATTACCAGCGGATGGACATTTGGAGCCGGTGCCGCTC
TGCAGATTCCCTTCGCTATGCAGATGGCCTACCGCTTCAA
TGGCATTGGCGTGACCCAGAATGTGCTGTACGAGAACCAG
AAGCTGATCGCCAACCAGTTCAACAGCGCCATCGGCAAGA
TTCAGGACAGCCTGAGTAGTACCGCCAGCGCTCTGGGAAA
GCTGCAGGATGTGGTCAACCAGAACGCTCAGGCCCTGAAC
ACCCTGGTTAAGCAGCTGAGCAGCAACTTCGGCGCCATCA
GTAGCGTGCTGAACGATATCCTGAGCCGCCTGGATAAGGT
GGAAGCCGAGGTGCAGATCGATCGCCTGATTACCGGACGC
CTGCAGTCCCTGCAGACCTATGTGACACAGCAGCTGATCC
GAGCCGCCGAGATTCGAGCTAGTGCTAATCTGGCCGCCAC
CAAGATGAGCGAATGTGTGCTGGGACAGAGCAAGCGCGTG
GACTTTTGCGGCAAGGGATACCACCTGATGAGCTTCCCAC
AGAGTGCTCCACACGGCGTGGTGTTTCTGCATGTGACCTA
CGTGCCCGCTCAAGAGAAGAATTTCACCACCGCTCCAGCC
ATCTGCCACGACGGAAAGGCCCATTTTCCACGCGAGGGCG
TGTTCGTTAGCAACGGCACTCATTGGTTCGTCACCCAGCG
CAACTTCTACGAGCCCCAGATCATCACCACCGACAACACC
TTCGTCAGCGGCAACTGCGACGTCGTGATCGGCATTGTGA
ACAACACCGTGTACGATCCACTGCAGCCCGAGCTGGACAG
CTTCAAAGAGGAACTGGACAAGTACTTTAAGAACCACACA
AGCCCCGACGTGGACCTGGGAGACATTAGCGGAATCAACG
CCAGCGTGGTCAACATCCAGAAAGAGATTGACCGCCTGAA
CGAGGTGGCCAAGAATCTGAACGAGAGCCTGATCGACCTG
CAAGAACTGGGCAAATACGAGCAGTACATTAAGTGGCCCT
GGTACATCTGGCTGGGCTTCATTGCCGGACTGATTGCCAT
CGTGATGGTCACCATTATGCTGTGCTGCATGACCAGTTGC
TGCAGCTGCCTGAAGGGATGCTGCAGTTGCGGAAGCTGCT
GCAAGTTCGACGAGGATGATAGCGAGCCAGTGCTGAAGGG
CGTCAAGCTGCACTACACC

In some embodiments the SARS-CoV-2 S Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 2.

In some embodiments the SARS-CoV-2 S Protein is encoded by a nucleotide sequence that is codon-optimized, such as a codon optimized variant of SEQ ID NO: 2.

### Angiotensin-converting Enzyme 2 (ACE2) Protein and Nucleic Acid

Various aspects of this disclosure incorporate a ACE2 protein. In a preferred embodiment the ACE2 Protein comprises or consists of the following amino acid sequence (UniProtKB—Q9BYF1 (ACE2_HUMAN); SEQ ID NO: 3):

MSSSSWLLLSLVAVTAAQSTIEEQAKTFLDKFNHEAEDLF
YQSSLASWNYNINITEENVQNMNNAGDKWSAFLKEQSTLA
QMYPLQEIQNLTVKLQLQALQQNGSSVLSEDKSKRINTIL
NTMSTIYSTGKVCNPDNPQECLLLEPGLNEIMANSLDYNE
RLWAWESWRSEVGKQLRPLYEEYVVLKNEMARANHYEDYG
DYWRGDYEVNGVDGYDYSRGQLIEDVEHTFEEIKPLYEHL
HAYVRAKLMNAYPSYISPIGCLPAHLLGDMWGRFWTNLYS
LTVPFGQKPNIDVTDAMVDQAWDAQRIFKEAEKFFVSVGL
PNMTQGFWENSMLTDPGNVQKAVCHPTAWDLGKGDFRILM
CTKVTMDDFLTAHHEMGHIQYDMAYAAQPFLLRNGANEGF
HEAVGEIMSLSAATPKHLKSIGLLSPDFQEDNETEINFLL
KQALTIVGTLPFTYMLEKWRWMVFKGEIPKDQWMKKWWEM
KREIVGVVEPVPHDETYCDPASLFHVSNDYSFIRYYTRTL
YQFQFQEALCQAAKHEGPLHKCDISNSTEAGQKLFNMLRL
GKSEPWTLALENVVGAKNMNVRPLLNYFEPLFTWLKDQNK
NSFVGWSTDWSPYADQSIKVRISLKSALGDKAYEWNDNEM
YLFRSSVAYAMRQYFLKVKNQMILFGEEDVRVANLKPRIS
ENFFVTAPKNVSDIIPRTEVEKAIRMSRSRINDAFRLNDN
SLEFLGIQPTLGPPNQPPVSIWLIVFGVVMGVIVVGIVIL
IFTGIRDRKKKNKARSGENPYASIDISKGENNPGFQNTDD
VQTSF

In some embodiments the ACE2 Protein comprises or consists of an amino acid sequence that is at least 90%, 91%, 92%, 93%, 94%, 95%, 96%, 97%, 98%, or 99% identical to SEQ ID NO: 3.

In some embodiments the ACE2 Protein comprises or consists of an amino acid sequence that has 1, 2, 3, 4, 5, 6, 7, 8, 9 or 10 amino acid changes relative to SEQ ID NO: 3. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 3.

In a preferred embodiment the ACE2 is encoded by a nucleotide sequence that comprises or consists of the following nucleotide sequence (SEQ ID NO: 4):

ATGTCAAGCTCTTCCTGGCTCCTTCTCAGCCTTGTTGCTG
TAACTGCTGCTCAGTCCACCATTGAGGAACAGGCCAAGAC
ATTTTTGGACAAGTTTAACCACGAAGCCGAAGACCTGTTC
TATCAAAGTTCACTTGCTTCTTGGAATTATAACACCAATA
TTACTGAAGAGAATGTCCAAAACATGAATAATGCTGGGGA
CAAATGGTCTGCCTTTTTAAAGGAACAGTCCACACTTGCC
CAAATGTATCCACTACAAGAAATTCAGAATCTCACAGTCA
AGCTTCAGCTGCAGGCTCTTCAGCAAAATGGGTCTTCAGT
GCTCTCAGAAGACAAGAGCAAACGGTTGAACACAATTCTA
AATACAATGAGCACCATCTACAGTACTGGAAAAGTTTGTA
ACCCAGATAATCCACAAGAATGCTTATTACTTGAACCAGG
TTTGAATGAAATAATGGCAAACAGTTTAGACTACAATGAG
AGGCTCTGGGCTTGGGAAAGCTGGAGATCTGAGGTCGGCA
AGCAGCTGAGGCCATTATATGAAGAGTATGTGGTCTTGAA
AAATGAGATGGCAAGAGCAAATCATTATGAGGACTATGGG
GATTATTGGAGAGGAGACTATGAAGTAAATGGGGTAGATG
GCTATGACTACAGCCGCGGCCAGTTGATTGAAGATGTGGA
ACATACCTTTGAAGAGATTAAACCATTATATGAACATCTT
CATGCCTATGTGAGGGCAAAGTTGATGAATGCCTATCCTT
CCTATATCAGTCCAATTGGATGCCTCCCTGCTCATTTGCT
TGGTGATATGTGGGGTAGATTTTGGACAAATCTGTACTCT
TTGACAGTTCCCTTTGGACAGAAACCAAACATAGATGTTA
CTGATGCAATGGTGGACCAGGCCTGGGATGCACAGAGAAT
ATTCAAGGAGGCCGAGAAGTTCTTTGTATCTGTTGGTCTT
CCTAATATGACTCAAGGATTCTGGGAAAATTCCATGCTAA
CGGACCCAGGAAATGTTCAGAAAGCAGTCTGCCATCCCAC
AGCTTGGGACCTGGGGAAGGGCGACTTCAGGATCCTTATG
TGCACAAAGGTGACAATGGACGACTTCCTGACAGCTCATC
ATGAGATGGGGCATATCCAGTATGATATGGCATATGCTGC
ACAACCTTTTCTGCTAAGAAATGGAGCTAATGAAGGATTC
CATGAAGCTGTTGGGGAAATCATGTCACTTTCTGCAGCCA
CACCTAAGCATTTAAAATCCATTGGTCTTCTGTCACCCGA
TTTTCAAGAAGACAATGAAACAGAAATAAACTTCCTGCTC
AAACAAGCACTCACGATTGTTGGGACTCTGCCATTTACTT
ACATGTTAGAGAAGTGGAGGTGGATGGTCTTTAAAGGGGA
AATTCCCAAAGACCAGTGGATGAAAAAGTGGTGGGAGATG
AAGCGAGAGATAGTTGGGGTGGTGGAACCTGTGCCCCATG
ATGAAACATACTGTGACCCCGCATCTCTGTTCCATGTTTC
TAATGATTACTCATTCATTCGATATTACACAAGGACCCTT
TACCAATTCCAGTTTCAAGAAGCACTTTGTCAAGCAGCTA
AACATGAAGGCCCTCTGCACAAATGTGACATCTCAAACTC
TACAGAAGCTGGACAGAAACTGTTCAATATGCTGAGGCTT
GGAAAATCAGAACCCTGGACCCTAGCATTGGAAAATGTTG
TAGGAGCAAAGAACATGAATGTAAGGCCACTGCTCAACTA
CTTTGAGCCCTTATTTACCTGGCTGAAAGACCAGAACAAG
AATTCTTTTGTGGGATGGAGTACCGACTGGAGTCCATATG
CAGACCAAAGCATCAAAGTGAGGATAAGCCTAAAATCAGC
TCTTGGAGATAAAGCATATGAATGGAACGACAATGAAATG
TACCTGTTCCGATCATCTGTTGCATATGCTATGAGGCAGT
ACTTTTTAAAAGTAAAAAATCAGATGATTCTTTTTGGGGA
GGAGGATGTGCGAGTGGCTAATTTGAAACCAAGAATCTCC
TTTAATTTCTTTGTCACTGCACCTAAAAATGTGTCTGATA
TCATTCCTAGAACTGAAGTTGAAAAGGCCATCAGGATGTC
CCGGAGCCGTATCAATGATGCTTTCCGTCTGAATGACAAC
AGCCTAGAGTTTCTGGGGATACAGCCAACACTTGGACCTC
CTAACCAGCCCCCTGTTTCCATATGGCTGATTGTTTTTGG
AGTTGTGATGGGAGTGATAGTGGTTGGCATTGTCATCCTG
ATCTTCACTGGGATCAGAGATCGGAAGAAGAAAAATAAAG
CAAGAAGTGGAGAAAATCCTTATGCCTCCATCGATATTAG
CAAAGGAGAAAATAATCCAGGATTCCAAAACACTGATGAT
GTTCAGACCTCCTTT

In some embodiments the ACE2 Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 4.

In some embodiments the ACE2 Protein is encoded by a nucleotide sequence that is codon-optimized, such as a codon optimized variant of SEQ ID NO: 4.

### Pseudotyped Lentiviral Vector Particles

This disclosure provides pseudotyped lentiviral vector particles bearing a SARS-CoV-2 S protein according to this disclosure. The lentivector can be integrative or non-integrative. The lentiviral vectors are pseudotyped lentiviral vectors (i.e. “lentiviral vector particles”) bearing a SARS-CoV-2 S protein.

The sequences of the original lentivirus encoding the lentiviral proteins may be essentially deleted from the genome of the vector or, when present, are modified, and particularly prevent expression of biologically active Pol antigen and optionally of further structural and/or accessory and/or regulatory proteins of the lentivirus.

Within the context of this disclosure, a “lentiviral vector” means a non-replicating vector for the transduction of a host cell with a transgene comprising cis-acting lentiviral RNA or DNA sequences, and requiring lentiviral proteins (e.g., Gag, Pol, and/or Env) that are provided in trans. The lentiviral vector contains cis-acting packaging sequences, but lacks expression of functional Gag, Pol, and Env proteins. The lentiviral vector may be present in the form of an RNA or DNA molecule, depending on the stage of production or development of the vector.

The lentiviral vector can be in the form of a recombinant DNA molecule, such as a plasmid. The lentiviral vector can be in the form of a lentiviral particle vector, such as an RNA molecule(s) within a complex of lentiviral and other proteins. Typically, lentiviral particle vectors, which correspond to modified or recombinant lentivirus particles, comprise a genome which is composed of two copies of single-stranded RNA. These RNA sequences can be obtained by transcription from a double-stranded DNA sequence inserted into a host cell genome (proviral vector DNA) or can be obtained from the transient expression of plasmid DNA (plasmid vector DNA) in a transformed host cell.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene.

The “vector genome” of the vector particles also comprises a polynucleotide or transgene that encodes a SARS-CoV-2 S protein.

In a particular embodiment, the transgene is also devoid of a polynucleotide encoding biologically active POL proteins. A biologically active POL antigen comprises the viral enzymes protease (RT), reverse transcriptase (RT and RNase H) and integrase (IN) produced by cleavage of the GAG-POL polyprotein. The POL antigen is not biologically active when the biological activity of at least one of these enzymes is not enabled. The biological activity is described with these enzymes in Fields (Virology—Vol 2 Chapter 60, pages 1889-1893 Edition 1996). In a particular embodiment, the polynucleotide or transgene in the vector genome is devoid of the functional pol gene, and especially does not contain a complete pol gene.

The vector genome as defined herein contains, apart from the polynucleotide or transgene that encodes a SARS-CoV-2 S protein placed under control of regulatory sequences, the sequences of the lentiviral genome which are non-coding regions, and are necessary to provide recognition signals for DNA or RNA synthesis and processing. These sequences are cis-acting sequences. The structure and composition of the vector genome used to prepare the lentiviral vectors of the invention are based on the principles described in the art. Examples of such lentiviral vectors are disclosed in (Zennou et al, 2000; Firat H. et al, 2002; VandenDriessche T. et al). Especially, minimum lentiviral gene delivery vectors can be prepared from a vector genome, which only contain, apart from the heterologous polynucleotide of therapeutic interest under control of regulatory sequences, the sequences of the lentiviral genome which are non-coding regions of the genome, necessary to provide recognition signals for DNA or RNA synthesis and processing. Hence, a vector genome can be a replacement vector in which all the viral protein coding sequences between the 2 long terminal repeats (LTRs) have been replaced by the polynucleotide of interest.

In a particular embodiment, the vector genome is defective for the expression of biologically functional Gag, and advantageously for biologically functional Pol and Env proteins. The 5′ LTR and 3′ LTR sequences of the lentivirus can be used in the vector genome. Preferably, the 3′-LTR is modified with respect to the 3′LTR of the original lentivirus, particularly in the U3 region. The 5′LTR can also be modified, particularly in its promoter region.

In a particular embodiment, the 3′ LTR sequence of the lentiviral vector genome is devoid of at least the activator (enhancer), and preferably also the promoter of the U3 region. In another particular embodiment, the 3′ LTR region is devoid of the U3 region (delta U3). In this respect, reference is made to WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator.

In a particular embodiment the vector genome is devoid of the coding sequences for Vif-, Vpr, Vpu- and Nef-accessory genes (for HIV-1 lentiviral vectors), or of their complete or functional genes.

In a particular embodiment, the vector genome of the lentiviral vector particles comprises, as an inserted cis-acting fragment, at least one polynucleotide consisting of or comprising the DNA flap. In a particular embodiment, the DNA flap is inserted upstream of the coding sequence for the label. Preferably, the DNA flap is located in an approximate central position in the vector genome. A DNA flap suitable for the invention can be obtained from a retrovirus, especially from a lentivirus, in particular a human lentivirus, or from a retrovirus-like organism such as retrotransposon. It can be alternatively obtained from the CAEV (Caprine Arthritis Encephalitis Virus) virus, the EIAV (Equine Infectious Anaemia Virus) virus, the Visna virus, the SIV (Simian Immunodeficiency Virus) virus or the FIV (Feline Immunodeficiency Virus) virus. The DNA flap can be prepared synthetically (chemical synthesis) or by amplification of the DNA, such as by polymerase chain reaction (PCR). In a more preferred embodiment, the DNA flap is obtained from an HIV retrovirus, for example HIV-1 or HIV-2 virus including any isolate of these two types.

The DNA flap (defined in Zennou V. et al., 2000, Cell vol 101, 173-185 or in WO 99/55892 and WO 01/27304, which are hereby incorporated by reference), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000).

Sequences of DNA flaps are well-known in the art, for example, in the above cited patent applications. They are preferably inserted as fragment comprising the DNA Flap into the vector genome in a position which is preferentially near the center of the vector genome. Alternatively, they can be inserted immediately upstream from the promoter controlling the expression of the polynucleotide of the invention. The fragments comprising the DNA flap, inserted in the vector genome can have a sequence of about 80 to about 200 bp, depending on its origin and preparation. According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it can be inserted as a longer sequence, especially when it is prepared as a PCR fragment. A particular appropriate polynucleotide comprising the structure providing the DNA flap is a 178-base pair polymerase chain reaction (PCR) fragment encompassing the cPPT and CTS regions of the HIV-1 DNA (Zennou et al 2000).

This PCR fragment can especially be derived from infective DNA clone of HIV-1 LAI, especially pLA13 of HIV1, as a fragment corresponding to the sequence from nucleotide 4793 to 4971. If appropriate, restriction sites are added to one or both extremities of the obtained fragment, for cloning. For example, Nar I restriction sites can be added to the 5′ extremities of primers used to perform the PCR reaction.

The DNA flap used in the genome vector and the Gag and Pol polyproteins of the lentiviral vector particles should originate from the same lentivirus sub-family or from the same retrovirus-like organism. Preferably, the other cis-activating sequences of the genome vector also originate from the same lentivirus or retrovirus-like organism, as the one providing the DNA flap.

In some embodiments, compositions are provided comprising at least 10⁵, 5×10⁵, 10⁶, 5×10⁶, 10⁷, 5×10⁷, 10⁸, 5×10⁸, 10⁹, 5×10⁹, or 10¹⁰ TU/ml of lentiviral vector particles pseudotyped with the SARS-CoV-2 S protein.

In a preferred embodiment the SARS-CoV-2 S Protein comprises or consists of the amino acid sequence of SEQ ID NO: 1. In some embodiments the SARS-CoV-2 S Protein comprises or consists of an amino acid sequence that is at least 90%, 91%, 92%, 93%, 94%, 95%, 96%, 97%, 98%, or 99% identical to SEQ ID NO: 1. In some embodiments the SARS-CoV-2 S Protein comprises or consists of an amino acid sequence that has 1, 2, 3, 4, 5, 6, 7, 8, 9 or 10 amino acid changes relative to SEQ ID NO: 1. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In some embodiments the SARS-CoV-2 S Protein is encoded by a nucleotide sequence that comprises or consists of SEQ ID NO: 2. In a some embodiments the SARS-CoV-2 S Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 2. In some embodiments the SARS-CoV-2 S Protein is encoded by a nucleotide sequence that is codon-optimized, such as a codon optimized variant of SEQ ID NO: 2.

In some embodiments the pseudotyped lentiviral vector particles further comprise a heterologous polynucleotide that encodes a label. In some embodiments the label is a protein that may be directly detected, such as a fluorescent protein. Examples of fluorescent proteins include green fluorescent protein (GFP), enhanced GFP (EGFP), superfolder GFP (sfGFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet). In some embodiments the label is an enzyme, which catalyses conversion of a substrate into a product that may be directly detected. Examples include luciferase and nano-luc. Skilled artisans will appreciate that many alternatives may be used, including gene products that are detected by other means.

In some embodiments the pseudotyped lentiviral vector particles further comprise a heterologous polynucleotide that encodes a recombinase, and in particular a Cre recombinase (Bacteriophage P1). In a preferred embodiment the Cre recombinase protein comprises or consists of SEQ ID NO: 14 (MSNLLTVHQNLPALPVDATSDEVRKNLMDMFRDRQAFSEHTWKMLLSVCRSWAAW CKLNNRKWFPAEPEDVRDYLLYLQARGLAVKTIQQHLGQLNMLHRRSGLPRPSDSNA VSLVMRRIRKENVDAGERAKQALAFERTDFDQVRSLMENSDRCQDIRNLAFLGIAYNT LLRIAEIARIRVKDISRTDGGRMLIHIGRTKTLVSTAGVEKALSLGVTKLVERWISVSGVA DDPNNYLFCRVRKNGVAAPSATSQLSTRALEGIFEATHRLIYGAKDDSGQRYLAWSGH SARVGAARDMARAGVSIPEIMQAGGWTNVNIVMNYIRNLDSETGAMVRLLEDGD). In some embodiments, the Cre recombinase protein is an amino acid sequence that is at least 60%, 70%, 80%, 85%, 90%, 91%, 92%, 93%, 94%, 95%, 96%, 97%, 98%, or 99% identical to SEQ ID NO: 14.

In a particular embodiment, the lentiviral vector particle is obtained by a method that includes using:
pCMV.SSARS-COV2, which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5608;

and one of the two following plasmids:
pTRIPdeltaU3-CMV-LucF-WPREm, which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5607 or
pTRIPdeltaU3.CMV.CRE, which was deposited at the Collection Nationale de Cultures de Microrganismes (CNCM), located at Institut Pasteur, 25-28 Rue du Docteur Roux 75724 Paris CEDEX 15 FRANCE on Sep. 15, 2021, under Reference Number CNCM I-5747.

In a particular embodiment, the lentiviral vector particle is obtained by a method that includes using one or both of the plasmids:
pCMV.SSARS-COV2, which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5608; and
pTRIPdeltaU3-CMV-LucF-WPREm, which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5607.

In a particular embodiment, the lentiviral vector particle is obtained by a method that includes using one or both, and in particular both, of the plasmids;
pCMV.SSARS-COV2, which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5608; and
pTRIPdeltaU3.CMV.CRE, which was deposited at the Collection Nationale de Cultures de Microrganismes (CNCM), located at Institut Pasteur, 25-28 Rue du Docteur Roux 75724 Paris CEDEX 15 FRANCE on Sep. 15, 2021, under Reference Number CNCM I-5747.

### Cells Expressing ACE2 Protein

In an aspect this disclosure provides mammalian cells expressing an ACE2 protein.

In a preferred embodiment the ACE2 Protein comprises or consists of the amino acid sequence of SEQ ID NO: 3. In some embodiments the ACE2 Protein comprises or consists of an amino acid sequence that is at least 90%, 91%, 92%, 93%, 94%, 95%, 96%, 97%, 98%, or 99% identical to SEQ ID NO: 3.

In some embodiments the ACE2 Protein comprises or consists of an amino acid sequence that has 1, 2, 3, 4, 5, 6, 7, 8, 9 or 10 amino acid changes relative to SEQ ID NO: 3. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 3.

In a preferred embodiment the ACE2 protein is encoded by a nucleotide sequence that comprises or consists of SEQ ID NO: 4. In some embodiments the ACE2 Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 4. In some embodiments the ACE2 Protein is encoded by a nucleotide sequence that is codon-optimized, such as a codon optimized variant of SEQ ID NO: 4.

A skilled artisan will appreciate that in certain embodiments any suitable mammalian cells known in the art may be used. In preferred embodiments the cells are human cells. In one embodiment the cells are 293T cells. In one embodiment the cells are HEK293T cells. In a preferred embodiment the 293T cells are from ATCC (ATCC® CRL-3216™).

Suitable methods of growing and maintaining 293T cells are well known in the art. In a nonlimiting example, 293T cells may be split every 2-3 days using DMEM medium supplemented with 10% fetal calf serum and 1% Penicillin streptomycin (complete medium).

In one embodiment the cells are HeLa cells.

In some embodiments the ACE2 protein is expressed from an endogenous gene in the genome of the mammalian cells.

In some embodiments the ACE2 protein is expressed from a heterologous coding sequence present on a plasmid in the cell. In some embodiments the ACE2 protein is expressed from a heterologous coding sequence integrated into the genome of the cell.

In some embodiments the ACE2 protein is expressed from a coding sequence present in a lentiviral vector that is integrated into the genome of the cell. In some embodiments the lentiviral vector is a pLV-Puro vector.

In a preferred embodiment, the cell line stably expressing hACE2 receptor (293T-hACE2) is the cell line deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5609.

In a preferred embodiment, cell line stably expressing hACE2 receptor expresses hACE2 receptor and GFP (HEK 293T_hACE2_eGFP) and is the cell line deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5611.

In some embodiments the cell line is obtained by a process that includes utilizing the lentiviral vector (pTRIPdeltaU3.hUBC-hACE2), which was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5610.

In a particular embodiment, cell line stably expressing hACE2 receptor further expresses nanolox (293T::hACE2::Nanolox) and is the cell line deposited at the Collection Nationale de Cultures de Microrganismes (CNCM), located at Institut Pasteur, 25-28 Rue du Docteur Roux 75724 Paris CEDEX 15 FRANCE on Sep. 15, 2021 under the Reference Number CNCM I-5746.

Nanolox is a nucleotide sequence consisting in the inverted nucleotide sequence of Nanoluc, flanked by inverted LOXP sequences (sequences SEQ ID NO: 12, or a nucleotide sequence that is at least 90%, 91%, 92%, 93%, 94%, 95%, 96%, 97%, 98%, or 99% identical to SEQ ID NO: 12, and SEQ ID NO: 13, or a nucleotide sequence that is at least 90%, 91%, 92%, 93%, 94%, 95%, 96%, 97%, 98%, or 99% identical to SEQ ID NO: 13). In particular, Nanolox comprises or consists of SEQ ID NO: 11. In some embodiments, the Nanolox nucleotide sequence comprises or consists of a nucleotide sequence that is at least 90%, 91%, 92%, 93%, 94%, 95%, 96%, 97%, 98%, or 99% identical to SEQ ID NO: 11.

In some embodiments, the Nanoluc protein comprises or consists of SEQ ID NO: 10:

MVFTLEDFVGDWRQTAGYNLDQVLEQGGVSSLFQNLGVSV
TPIQRIVLSGENGLKIDIHVIIPYEGLSGDQMGQIEKIFK
VVYPVDDHHFKVILHYGTLVIDGVTPNMIDYFGRPYEGIA
VFDGKKITVTGTLWNGNKIIDERLINPDGSLLFRVTINGV
TGWRLCERILA.

In some embodiments, the Nanoluc protein comprises or consists of an amino acid sequence that is at least 90%, 91%, 92%, 93%, 94%, 95%, 96%, 97%, 98%, or 99% identical to SEQ ID NO: 10.

In some embodiments the cell line is obtained by a process that includes utilizing the lentiviral vector (pTRIPdeltaU3.UBC.nanoluc LoxP), which was deposited at the Collection Nationale de Cultures de Microrganismes (CNCM), located at Institut Pasteur, 25-28 Rue du Docteur Roux 75724 Paris CEDEX 15 FRANCE on Sep. 15, 2021, under Reference Number CNCM I-5748.

### Compositions and Kits

In another aspect this disclosure provides compositions and kits. The composition or a kit may comprise a pseudotyped lentiviral vector particle bearing a SARS-CoV-2 S protein, according to this disclosure; and a mammalian cell expressing an Angiotensin-converting Enzyme 2 (ACE2) protein, according to this disclosure.

In some embodiments the SARS-CoV-2 S Protein comprises or consists of SEQ ID NO: 1. In some embodiments the SARS-CoV-2 S Protein comprises or consists of an amino acid sequence that is at least 90%, 91%, 92%, 93%, 94%, 95%, 96%, 97%, 98%, or 99% identical to SEQ ID NO: 1.

In some embodiments the SARS-CoV-2 S Protein comprises or consists of an amino acid sequence that has 1, 2, 3, 4, 5, 6, 7, 8, 9 or 10 amino acid changes relative to SEQ ID NO: 1. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In a preferred embodiment the SARS-CoV-2 S Protein is encoded by a nucleotide sequence that comprises or consists of SEQ ID NO: 2. In some embodiments the SARS-CoV-2 S Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 2. In some embodiments the SARS-CoV-2 S Protein is encoded by a nucleotide sequence that is codon-optimized, such as a codon optimized variant of SEQ ID NO: 2.

In some embodiments the pseudotyped lentiviral vector particles further comprise a heterologous polynucleotide that encodes a label. In some embodiments the label is a protein that may be directly detected, such as a fluorescent protein. Examples of fluorescent proteins include green fluorescent protein (GFP), enhanced GFP (EGFP), superfolder GFP (sfGFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet). In some embodiments the label is an enzyme, which catalyses conversion of a substrate into a product that may be directly detected. Examples include luciferase and nano-luc. Skilled artisans will appreciate that many alternatives may be used, including gene products that are detected by other means.

In some embodiments the pseudotyped lentiviral vector particles further comprise a heterologous polynucleotide that encodes a recombinase, and in particular a Cre recombinase (Bacteriophage P1). In a preferred embodiment the Cre recombinase protein comprises or consists of SEQ ID NO: 14 (MSNLLTVHQNLPALPVDATSDEVRKNLMDMFRDRQAFSEHTWKMLLSVCRSWAAW CKLNNRKWFPAEPEDVRDYLLYLQARGLAVKTIQQHLGQLNMLHRRSGLPRPSDSNA VSLVMRRIRKENVDAGERAKQALAFERTDFDQVRSLMENSDRCQDIRNLAFLGIAYNT LLRIAEIARIRVKDISRTDGGRMLIHIGRTKTLVSTAGVEKALSLGVTKLVERWISVSGVA DDPNNYLFCRVRKNGVAAPSATSQLSTRALEGIFEATHRLIYGAKDDSGQRYLAWSGH SARVGAARDMARAGVSIPEIMQAGGWTNVNIVMNYIRNLDSETGAMVRLLEDGD). In some embodiments, the Cre recombinase protein is an amino acid sequence that is at least 60%, 70%, 80%, 85%, 90%, 91%, 92%, 93%, 94%, 95%, 96%, 97%, 98%, or 99% identical to SEQ ID NO: 14.

In a preferred embodiment the ACE2 Protein comprises or consists of the amino acid sequence of SEQ ID NO: 3. In some embodiments the ACE2 Protein comprises or consists of an amino acid sequence that is at least 90%, 91%, 92%, 93%, 94%, 95%, 96%, 97%, 98%, or 99% identical to SEQ ID NO: 3.

In some embodiments the ACE2 Protein comprises or consists of an amino acid sequence that has 1, 2, 3, 4, 5, 6, 7, 8, 9 or 10 amino acid changes relative to SEQ ID NO: 3. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 3.

In a preferred embodiment the ACE2 protein is encoded by a nucleotide sequence that comprises or consists of SEQ ID NO: 4. In some embodiments the ACE2 Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 4. In some embodiments the ACE2 Protein is encoded by a nucleotide sequence that is codon-optimized, such as a codon optimized variant of SEQ ID NO: 4.

A skilled artisan will appreciate that in certain embodiments any suitable mammalian cells known in the art may be used. In preferred embodiments the cells are human cells. In one embodiment the cells are 293T cells. In one embodiment the cells are HEK293T cells. In a preferred embodiment the 293T cells are from ATCC (ATCC® CRL-3216™).

Suitable methods of growing and maintaining 293T cells are well known in the art. In a nonlimiting example, 293T cells may be split every 2-3 days using DMEM medium supplemented with 10% fetal calf serum and 1% Penicillin streptomycin (complete medium).

In one embodiment the cells are HeLa cells.

In some embodiments the ACE2 protein is expressed from an endogenous gene in the genome of the mammalian cells.

In some embodiments the ACE2 protein is expressed from a heterologous coding sequence present on a plasmid in the cell. In some embodiments the ACE2 protein is expressed from a heterologous coding sequence integrated into the genome of the cell.

In some embodiments the ACE2 protein is expressed from a coding sequence present in a lentiviral vector that is integrated into the genome of the cell. In some embodiments the lentiviral vector is a pLV-Puro vector.

In some embodiments the mammalian cells expressing an ACE2 protein are human cells. In one embodiment the cells are 293T cells. In one embodiment the cells are HEK293T cells. In a preferred embodiment the 293T cells are from ATCC (ATCC® CRL-3216™).

Suitable methods of growing and maintaining 293T cells are well known in the art. In a nonlimiting example, 293T cells may be split every 2-3 days using DMEM medium supplemented with 10% fetal calf serum and 1% Penicillin streptomycin (complete medium).

In one embodiment the cells are HeLa cells.

In some embodiments the ACE2 protein is expressed from an endogenous gene in the genome of the mammalian cells.

In some embodiments the ACE2 protein is expressed from a heterologous coding sequence present on a plasmid in the cell. In some embodiments the ACE2 protein is expressed from a heterologous coding sequence integrated into the genome of the cell.

In some embodiments the ACE2 protein is expressed from a coding sequence present in a lentiviral vector that is integrated into the genome of the cell. In some embodiments the lentiviral vector is a pLV-Puro vector.

In some embodiments the mammalian cell further expresses the serine protease TMPRSS2.

In some embodiments, a composition, kit or system according to the invention, comprises:
a pseudotyped lentiviral vector particle bearing a SARS-CoV-2 S protein, in particular a SARS-CoV-2 S protein having an amino acid sequence at least 95% identical to SEQ ID NO: 1, the pseudotyped lentiviral vector particle further comprising a heterologous polynucleotide that encodes a recombinase, and in particular a Cre recombinase (Bacteriophage P1), more particularly a Cre recombinase protein comprising or consisting of a sequence that is at least 90%, 91%, 92%, 93%, 94%, 95%, 96%, 97%, 98%, or 99% identical to SEQ ID NO: 14; and
a mammalian cell expressing an Angiotensin-converting Enzyme 2 (ACE2) protein and comprising a nanolox nucleotide sequence, in particular a nanolox nucleotide sequence comprising or consisting of a sequence that is at least 90%, 91%, 92%, 93%, 94%, 95%, 96%, 97%, 98%, or 99% identical to SEQ ID NO: 11.

In some embodiments the composition or the kit further comprises a human serum. The human serum may be from a patient having a known SARS-CoV-2 infection. The human serum may be from a patient suspected of having a SARS-CoV-2 infection. The human serum may be from a patient who has a SARS-CoV-2 infection and manifests symptoms of Covid19. The human serum may be from a patient who has a SARS-CoV-2 infection and does not manifest symptoms of Covid19. The human serum may be from a patient who had a SARS-CoV-2 infection and manifested symptoms of Covid19 but has since recovered.

In some embodiments the composition or the kit further comprises a SARS-CoV-2 S protein binding agent and/or an ACE2 binding agent.

The binding agent may be an antibody.

The antibody may be an antibody generated by a patient's immune system following infection with SARS-CoV-2. Alternatively, the antibody may be an antibody from any other source known in the art. In some embodiments the antibody is generated by introducing (e.g., by injection) a SARS-CoV-2 S protein or antigenic fragment thereof, or an ACE2 protein or antigenic fragment thereof into a mammal.

In some embodiments the antibody is a polyclonal antibody. In some embodiments the antibody is a monoclonal antibody. In some embodiments the antibody is an IgG antibody. In some embodiments the antibody is an IgM antibody.

In some embodiments the antibody is a chimeric antibody and/or fragment of an antibody (Fab, Fv, scFv) directed against the SARS-CoV-2 S protein or the ACE2 protein.

For the purposes of the present disclosure, the expression chimeric antibody is understood to mean, in relation to an antibody of a particular animal species or of a particular class of antibody, an antibody of a given animal species and/or class of antibody comprising all or part of a heavy chain and/or of a light chain of an antibody of another animal species and/or of another class of antibody.

In some embodiments, purified proteins are used to produce antibodies by conventional techniques. In some embodiments, recombinant or synthetic proteins or peptides are used to produce antibodies by conventional techniques.

Antibodies can be synthetic, semi-synthetic, monoclonal, or polyclonal and can be made by techniques well known in the art. Such antibodies specifically bind to proteins and polypeptides via the antigen-binding sites of the antibody (as opposed to non-specific binding). Purified or synthetic proteins and peptides can be employed as immunogens in producing antibodies immunoreactive therewith. The proteins and peptides contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes can be identified by any of the methods known in the art. Such epitopes or variants thereof can be produced using techniques well known in the art such as solid-phase synthesis, chemical or enzymatic cleavage of a polypeptide, or using recombinant DNA technology.

Antibodies are defined to be specifically binding if they bind proteins or polypeptides with a Ka of greater than or equal to about 10⁷ M⁻¹. Affinities of binding partners or antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, alpaca, camels, rabbits, mice, or rats, using procedures that are well known in the art. In general, a purified protein or polypeptide that is appropriately conjugated is administered to the host animal typically through parenteral injection. The immunogenicity can be enhanced through the use of an adjuvant, for example, Freund's complete or incomplete adjuvant. Following booster immunizations, small samples of serum are collected and tested for reactivity to proteins or polypeptides. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980.

For example, the host animals, such as mice, can be injected intraperitoneally at least once and preferably at least twice at about 3 week intervals with isolated and purified proteins or conjugated polypeptides, for example a peptide comprising or consisting of the specific amino acids set forth above. Mouse sera are then assayed by conventional dot blot technique or antibody capture (ABC) to determine which animal is best to fuse. Approximately two to three weeks later, the mice are given an intravenous boost of the protein or polypeptide. Mice are later sacrificed, and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as a labeled protein or polypeptide, is added to each well followed by incubation. Positive wells can be subsequently detected. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of such antibodies, which can be produced by conventional techniques, are also encompassed by the present invention. Examples of such fragments include, but are not limited to, Fab and F(ab′)2 fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies can be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806.

Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Publication No. WO 87/02671; Akira, et al. European Patent Application 0184187; Taniguchi, M., European Patent Application 0171496; Morrison et al. European Patent Application 0173494; Neuberger et al. PCT International Publication No. WO 86/01533; Cabilly et al. U.S. Pat. No. 4,816,567; Cabilly et al. European Patent Application 0125023; Better et al., Science 240:1041 1043, 1988; Liu et al., PNAS 84:3439 3443, 1987; Liu et al., J. Immunol. 139:3521 3526, 1987; Sun et al. PNAS 84:214 218, 1987; Nishimura et al., Canc. Res. 47:999 1005, 1987; Wood et al., Nature 314:446 449, 1985; and Shaw et al., J. Natl. Cancer Inst. 80:1553 1559, 1988); Morrison, S. L., Science 229:1202 1207, 1985; Oi et al., BioTechniques 4:214, 1986; Winter U.S. Pat. No. 5,225,539; Jones et al., Nature 321:552 525, 1986; Verhoeyan et al., Science 239:1534, 1988; and Beidler et al., J. Immunol. 141:4053 4060, 1988.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

In one embodiment, the invention encompasses single-domain antibodies (sdAb), also known as NANOBODIES. A sdAb is a fragment consisting of a single monomeric variable antibody domain that can bind selectively to a specific antigen.

In one embodiment, the sdAbs are from heavy-chain antibodies found in camelids (VHH fragments), or cartilaginous fishes (VNAR fragments), or are obtained by splitting dimeric variable domains into monomers.

In some embodiments the antibody is a patient antibody. In some embodiments the antibody is present in isolated patient serum.

In some embodiments the antibody is labeled.

Preferably, label is selected from a chemiluminescent label, an enzyme label, a fluorescence label, and a radioactive (e.g., iodine) label. In a preferred embodiment the secondary antibody is a labeled antibody or antibody fragment that binds to human immunoglobulins.

Preferred labels include a fluorescent label, such as FITC, a chromophore label, an affinity-ligand label, an enzyme label, such as alkaline phosphatase, horseradish peroxidase, or p galactosidase, an enzyme cofactor label, a hapten conjugate label, such as digoxigenin or dinitrophenyl, a Raman signal generating label, a magnetic label, a spin label, an epitope label, such as the FLAG or HA epitope, a luminescent label, a heavy atom label, a nanoparticle label, an electrochemical label, a light scattering label, a spherical shell label, semiconductor nanocrystal label, wherein the label can allow visualization with or without a secondary detection molecule.

Preferred labels include suitable enzymes such as horseradish peroxidase, alkaline phosphatase, beta-galactosidase, or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, as well as others known in the art; a luminescent material such as luminol; light scattering or plasmon resonant materials such as gold or silver particles or quantum dots; or radioactive material include ¹⁴C, ¹²³I, ¹²⁴I, ¹²⁵I, ³²P, ³³P, ³⁵S, or ³H.

In one embodiment, the secondary antibody or an antibody fragment that binds to human immunoglobulins binds specifically to IgG, IgA, and IgM. In one embodiment, the antibody or an antibody fragment that binds to human immunoglobulins binds specifically to IgG, IgA, or IgM.

The term “antibody” or “antibodies” is meant to include polyclonal antibodies, monoclonal antibodies, fragments thereof, such as F(ab′)2 and Fab fragments, single-chain variable fragments (scFvs), single-domain antibody fragments (VHHs or Nanobodies), bivalent antibody fragments (diabodies), as well as any recombinantly and synthetically produced binding partners.

In some embodiments, the antibody is a camelid VHH.

In a preferred embodiment, the antibody is an alpaca VHH.

In some embodiments the composition or the kit further comprises reagents for visualizing the label encoded by the lentiviral vector particle.

Uses of the compositions and kits are also provided.

The use of a lentivector particle according to this disclosure and/or a composition, kit or system according to this disclosure, to detect the presence of neutralizing antibodies against SARS-CoV-2 in a sample comprising antibodies is provided.

A lentivector particle according to this disclosure and/or a composition, kit or system according to this disclosure for use in detecting the presence of neutralizing antibodies against SARS-CoV-2 in a sample comprising antibodies is provided.

### Methods of Assaying for the Presence of Neutralizing Antibodies Against a SARS-CoV-2 S Protein

In another aspect, this disclosure provides methods of assaying for the presence of neutralizing antibodies against a SARS-CoV-2 S protein in a sample comprising antibodies. In some embodiments the sample is a human serum sample. In some embodiments the method further comprises fractionating the sample, such as a serum sample, and performing the assay using a fraction of the sample.

The sample may be from a patient having a known SARS-CoV-2 infection. The sample may be from a patient suspected of having a SARS-CoV-2 infection. The sample may be from a patient who has a SARS-CoV-2 infection and manifests symptoms of Covid19. The sample may be from a patient who has a SARS-CoV-2 infection and does not manifest symptoms of Covid19. The sample may be from a patient who had a SARS-CoV-2 infection and manifested symptoms of Covid19 but has since recovered.

The methods may comprise a) providing pseudotyped lentiviral vector particles bearing a SARS-CoV-2 S protein and comprising a heterologous polynucleotide that encodes a label; b) providing mammalian cells expressing an ACE2 protein; c) contacting the pseudotyped lentiviral vector particles bearing a SARS-CoV-2 S protein with a sample comprising antibodies; d) contacting the mammalian cells expressing an ACE2 protein with the pseudotyped lentiviral vector particles bearing a SARS-CoV-2 S protein; and e) assaying for the presence of the label in the mammalian cells. Detecting the presence of the label indicates that the coding sequence for the label has been delivered to the mammalian cell by the lentiviral vector particle following binding of the SARS-CoV-2 S protein on the particle with the ACE2 protein on the surface of the mammalian cell. In this way, detecting the presence of the label is an indication of this binding. If a sample comprising antibodies contains neutralizing antibodies against the SARS-CoV-2 S protein the antibodies will disrupt this interaction and reduce or even eliminate the presence of the label in the cells in the assay.

In some embodiments, c) and d) occur sequentially. It is understood that because the pseudotyped lentiviral vector particles bearing a SARS-CoV-2 S protein are contacted in c) with a sample comprising antibodies, the antibodies may still be present when d) is performed at a later time in a sequential method. In some embodiments unbound antibodies are removed between c) and the start of d). In a preferred embodiment they are not. In some embodiments, c) and d) occur simultaneously. However, in a preferred embodiment c) occurs before d).

In some embodiments, c) comprises incubating the pseudotyped lentiviral vector particles bearing a SARS-CoV-2 S protein with the sample comprising antibodies for at least 15 minutes prior to performing d). In some embodiments, c) comprises incubating the pseudotyped lentiviral vector particles bearing a SARS-CoV-2 S protein with the sample comprising antibodies for from 30 to 60 minutes prior to performing d). In some embodiments, c) comprises incubating the pseudotyped lentiviral vector particles bearing a SARS-CoV-2 S protein with the sample comprising antibodies for at least 5, 10, 15, 20, 25, 30, 45, 60, 90, 120, or 180 minutes prior to performing d). In some embodiments, c) comprises incubating the pseudotyped lentiviral vector particles bearing a SARS-CoV-2 S protein with the sample comprising antibodies for from 10 to 20 minutes, from 15 to 30 minutes, from 20 to 60 minutes, from 30 to 90 minutes, or from 60 to 180 minutes prior to performing d).

In some embodiments, d) comprises incubating the mammalian cells expressing an ACE2 protein with the pseudotyped lentiviral vector particles bearing a SARS-CoV-2 S protein for from 48 to 72 hours. In some embodiments, d) comprises incubating the mammalian cells expressing an ACE2 protein with the pseudotyped lentiviral vector particles bearing a SARS-CoV-2 S protein for from 12 to 24 hours, from 18 to 36 hours, from 24 to 48 hours, from 48 to 72 hours or from 48 to 96 hours.

In some embodiments, the mammalian cells in d) are adhered to a solid support.

In some embodiments, the mammalian cells in d) are in a suspension culture.

In another embodiment, the methods of the invention may comprise a) providing pseudotyped lentiviral vector particles bearing a SARS-CoV-2 S protein and comprising a heterologous polynucleotide that encodes a recombinase, and in particular a Cre recombinase, more particularly a Cre recombinase comprising or consisting of an amino acid sequence that is at least 60%, 70%, 80%, 85%, 90%, 91%, 92%, 93%, 94%, 95%, 96%, 97%, 98%, or 99% identical to SEQ ID NO: 14; b) providing mammalian cells expressing an ACE2 protein and comprising a nanolox nucleotide sequence, the nanolox sequence being in particular a nanolox nucleotide sequence comprising or consisting of a sequence that is at least 90%, 91%, 92%, 93%, 94%, 95%, 96%, 97%, 98%, or 99% identical to SEQ ID NO: 11; c) contacting the pseudotyped lentiviral vector particles bearing a SARS-CoV-2 S protein with a sample comprising antibodies; d) contacting the mammalian cells expressing an ACE2 protein with the pseudotyped lentiviral vector particles bearing a SARS-CoV-2 S protein and comprising the heterologous polynucleotide that encodes the recombinase; and e) assaying for the presence of the expression of the Nanoluc protein encoded by the Nanolox sequence in the mammalian cells. Detecting the presence of the expression of Nanoluc indicates that the Cre recombinase gene has been delivered to the mammalian cell by the lentiviral vector particle following binding of the SARS-CoV-2 S protein on the particle with the ACE2 protein on the surface of the mammalian cell. In this way, detecting the presence of Nanoluc in the cell is an indication of this binding. If a sample comprising antibodies contains neutralizing antibodies against the SARS-CoV-2 S protein the antibodies will disrupt this interaction and reduce or even eliminate the presence of the Nanoluc in the cells in the assay. Indeed, as the sequence encoding the Nanoluc is reversed in the Nanolox sequence, and accordingly in the mammalian cell, the Nanoluc is not expressed. However, when the Cre recombinase is delivered to the mammalian cell encoding the Nanolox sequence by the lentiviral vector particle, the recombinase will recognize the inverted LOXP sequences flanking the reversed sequence of the Nanoluc and will reverse the Nanolox, allowing the sequence coding for the Nanoluc to be in the appropriate direction, which will allow its expression in the mammalian cells.

By “the sequence encoding the Nanoluc is reversed”, it is intended to mean that the nanoluc ORF (Open Reading Frame) sequence is reversed compared to the direction into which the promotor controlling expression of the sequence performs transcription.

By “inverted LOXP sequences flanking the reversed sequence of the Nanoluc” it is intended LOXP sequences being in opposite directions. Indeed, the Cre recombinase catalyzes the site specific recombination event between the two loxP sites. Both 13 bp repeat sequences on a single loxP site are recognized and bound by a Cre protein, forming a dimer. The two loxP sites, which are in opposite direction, then align in a parallel orientation, allowing the four Cre proteins to form a tetramer. A double-strand DNA break occurs within the core spacer of each loxP site and the two strands are ligated, resulting in the inversion of the reversed Nanoluc sequence comprised between the two loxP sites, i.e. the Nanoluc sequence can be transcribed by its promoter.

c) and d) may occur sequentially. It is understood that because the pseudotyped lentiviral vector particles bearing a SARS-CoV-2 S protein are contacted in c) with a sample comprising antibodies, the antibodies may still be present when d) is performed at a later time in a sequential method. In some embodiments unbound antibodies are removed between c) and the start of d). In a preferred embodiment they are not. In some embodiments, c) and d) occur simultaneously. However, c) may occur before d).

In some embodiments, c) comprises incubating the pseudotyped lentiviral vector particles bearing a SARS-CoV-2 S protein with the sample comprising antibodies for at least 15 minutes prior to performing d). In some embodiments, c) comprises incubating the pseudotyped lentiviral vector particles bearing a SARS-CoV-2 S protein with the sample comprising antibodies for from 30 to 60 minutes prior to performing d). In some embodiments, c) comprises incubating the pseudotyped lentiviral vector particles bearing a SARS-CoV-2 S protein with the sample comprising antibodies for at least 5, 10, 15, 20, 25, 30, 45, 60, 90, 120, or 180 minutes prior to performing d). In some embodiments, c) comprises incubating the pseudotyped lentiviral vector particles bearing a SARS-CoV-2 S protein with the sample comprising antibodies for from 10 to 20 minutes, from 15 to 30 minutes, from 20 to 60 minutes, from 30 to 90 minutes, or from 60 to 180 minutes prior to performing d).

In some embodiments, d) comprises incubating the mammalian cells expressing an ACE2 protein with the pseudotyped lentiviral vector particles bearing a SARS-CoV-2 S protein for from 48 to 72 hours. In some embodiments, d) comprises incubating the mammalian cells expressing an ACE2 protein with the pseudotyped lentiviral vector particles bearing a SARS-CoV-2 S protein for from 12 to 24 hours, from 18 to 36 hours, from 24 to 48 hours, from 48 to 72 hours or from 48 to 96 hours.

In some embodiments, the mammalian cells in d) are adhered to a solid support.

In some embodiments, the mammalian cells in d) are in a suspension culture.

The method implementing the CRE-Nanolox system unexpectedly demonstrated a superior signal/noise ratio compared to a method implementing pseudotyped lentiviral vector particles bearing a SARS-CoV-2 S protein and comprising a heterologous polynucleotide that encodes a label, and in particular that encodes a luciferase when a seroneutralisation test is performed as described above.

In some embodiments the SARS-CoV-2 S Protein comprises or consists of SEQ ID NO: 1. In some embodiments the SARS-CoV-2 S Protein comprises or consists of an amino acid sequence that is at least 90%, 91%, 92%, 93%, 94%, 95%, 96%, 97%, 98%, or 99% identical to SEQ ID NO: 1.

In some embodiments the SARS-CoV-2 S Protein comprises or consists of an amino acid sequence that has 1, 2, 3, 4, 5, 6, 7, 8, 9 or 10 amino acid changes relative to SEQ ID NO: 1. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In a preferred embodiment the SARS-CoV-2 S Protein is encoded by a nucleotide sequence that comprises or consists of SEQ ID NO: 2. In some embodiments the SARS-CoV-2 S Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 2. In some embodiments the SARS-CoV-2 S Protein is encoded by a nucleotide sequence that is codon-optimized, such as a codon optimized variant of SEQ ID NO: 2.

In some embodiments the pseudotyped lentiviral vector particles further comprise a heterologous polynucleotide that encodes a label. In some embodiments the label is a protein that may be directly detected, such as a fluorescent protein. Examples of fluorescent proteins include green fluorescent protein (GFP), enhanced GFP (EGFP), superfolder GFP (sfGFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet). In some embodiments the label is an enzyme, which catalyses conversion of a substrate into a product that may be directly detected. Examples include luciferase and nano-luc. Skilled artisans will appreciate that many alternatives may be used, including gene products that are detected by other means.

In a preferred embodiment the ACE2 Protein comprises or consists of the amino acid sequence of SEQ ID NO: 3. In some embodiments the ACE2 Protein comprises or consists of an amino acid sequence that is at least 90%, 91%, 92%, 93%, 94%, 95%, 96%, 97%, 98%, or 99% identical to SEQ ID NO: 3.

In some embodiments the ACE2 Protein comprises or consists of an amino acid sequence that has 1, 2, 3, 4, 5, 6, 7, 8, 9 or 10 amino acid changes relative to SEQ ID NO: 3. In some embodiments the SARS-CoV-2 S Protein comprises or consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 3.

In a preferred embodiment the ACE2 protein is encoded by a nucleotide sequence that comprises or consists of SEQ ID NO: 4. In some embodiments the ACE2 Protein is encoded by a nucleotide sequence that is at least 60%, 70%, 80%, 85%, 90%, 91%, 92%, 93%, 94%, 95%, 96%, 97%, 98%, or 99% identical to SEQ ID NO: 4. In some embodiments the ACE2 Protein is encoded by a nucleotide sequence that is codon-optimized, such as a codon optimized variant of SEQ ID NO: 4.

A skilled artisan will appreciate that in certain embodiments any suitable mammalian cells known in the art may be used. In preferred embodiments the cells are human cells. In one embodiment the cells are 293T cells. In one embodiment the cells are HEK293T cells. In a preferred embodiment the 293T cells are from ATCC (ATCC® CRL-3216™).

Suitable methods of growing and maintaining 293T cells are well known in the art. In a nonlimiting example, 293T cells may be split every 2-3 days using DMEM medium supplemented with 10% fetal calf serum and 1% Penicillin streptomycin (complete medium).

In one embodiment the cells are HeLa cells.

In some embodiments the ACE2 protein is expressed from an endogenous gene in the genome of the mammalian cells.

In some embodiments the ACE2 protein is expressed from a heterologous coding sequence present on a plasmid in the cell. In some embodiments the ACE2 protein is expressed from a heterologous coding sequence integrated into the genome of the cell.

In some embodiments the ACE2 protein is expressed from a coding sequence present in a lentiviral vector that is integrated into the genome of the cell. In some embodiments the lentiviral vector is a pLV-Puro vector.

In some embodiments the mammalian cells expressing an ACE2 protein are human cells. In one embodiment the cells are 293T cells. In one embodiment the cells are HEK293T cells. In a preferred embodiment the 293T cells are from ATCC (ATCC® CRL-3216™).

Suitable methods of growing and maintaining 293T cells are well known in the art. In a nonlimiting example, 293T cells may be split every 2-3 days using DMEM medium supplemented with 10% fetal calf serum and 1% Penicillin streptomycin (complete medium).

In one embodiment the cells are HeLa cells.

In some embodiments the ACE2 protein is expressed from an endogenous gene in the genome of the mammalian cells.

In some embodiments the ACE2 protein is expressed from a heterologous coding sequence present on a plasmid in the cell. In some embodiments the ACE2 protein is expressed from a heterologous coding sequence integrated into the genome of the cell.

In some embodiments the ACE2 protein is expressed from a coding sequence present in a lentiviral vector that is integrated into the genome of the cell. In some embodiments the lentiviral vector is a pLV-Puro vector.

In some embodiments the mammalian cell further expresses the serine protease TMPRSS2.

In some embodiments assaying for the presence of the label in the mammalian cells comprises measuring the level of the label in the mammalian cells. In some embodiments this is done by a process including measuring, directly or indirectly, the total label present in a population of cells, such as a population of cells growing in a well of a plate or in a defined area of a well of a plate. In some embodiments this is done by a process including measuring the proportion of cells among a population of cells that express at least a threshold level of the label.

In some embodiments the level of the label is less than or equal to a pre-determined threshold or a measured control value, indicating the presence of neutralizing antibodies against a SARS-CoV-2 S protein in a sample comprising antibodies. In some embodiments the level of the label is equal to or greater than a pre-determined threshold or a measured control value, indicating the absence of neutralizing antibodies against a SARS-CoV-2 S protein in a sample comprising antibodies.

### EXAMPLES

### Example 1. Materials and Methods

A. Cohorts

Pre-epidemic sera originated from two pre-epidemic healthy donors' sources: 200 sera from the Diagmicoll cohort collection of ICAReB platform29 approved by CPP Ile-de-France I, sampled before November 2019. 200 anonymized samples from blood donors recruited in March 2017 at the Val d'Oise sites of Etablissement Français du Sang (EFS, the French blood agency). The ICAReB platform (BRIF code no BB-0033-00062) of Institut Pasteur collects and manages bioresources following ISO 9001 and NF S 96-900 quality standards 29.

COVID-19 cases were from included at Hôpital Bichat-Claude-Bernard in the French COVID-19 cohort. Some of the patients have been previously described 24. Each participant provided written consent to participate to the study, which was approved by the regional investigational review board (IRB; Comité de Protection des Personnes Ile-de-France VII, Paris, France) and performed according to the European guidelines and the Declaration of Helsinki.

Pauci-symptomatic individuals: On Feb. 24, 2020, a patient from Crepy-en-Valois (Oise region, northern France) was admitted to a hospital in Paris with confirmed SARS-CoV-2 infection. As part of an epidemiological investigation around this case, a cluster of COVID-19 cases, based around a high school with an enrolment of 1200 pupils, was identified. On March 3-4, students at the high school, their parents, teachers and staff (administrative staff, cleaners, catering staff) were invited to participate to the investigation. A 5 mL blood sample was taken from 209 individuals who reported fever or mild respiratory symptoms (cough or dyspnea) since mid-January 2020. The median age was 18 years (interquartile range: 17-45), and 65% were female.

Samples from blood donors were collected by EFS (Lille, France) in Clermont (Oise) on March 20 and Noyon (Oise) on March 24, both cities are located at 60 kilometers from Crepy-en-Valois.

All sera were heat-inactivated 30-60 min at 56° C., aliquoted and conserved at 4° C. for short term use or frozen.

B. ELISA-N

A codon-optimized nucleotide fragment encoding full length nucleoprotein was synthetized and cloned into pETM11 expression vector (EMBL). The His-tagged SARS-CoV-2 N protein was bacterially expressed in E. coli BL21 (DE3) and purified as a soluble dimeric protein by affinity purification using a Ni-NTA Protino column (Macherey Nagel) and gel filtration using a Hiload 16/60 superdex 200 pg column (HE Healthcare). 96-well ELISA plates were coated overnight with N in PBS (50 ng/well in 50 μl). After washing 4 times with PBS-0.1% Tween 20 (PBST), 100 μl of diluted sera (1:200) in PBST-3% milk were added and incubated 1 h at 37° C. After washing 3 times with PBST, plates were incubated with 8,000-fold diluted peroxydase-conjugated goat anti-human IgG (Southern Biotech) for 1 h. Plates were revealed by adding 100 μl of HRP chromogenic substrate (TMB, Eurobio Scientific) after 3 washing steps in PBST. After 30 min incubation, optical densities were measured at 405 nm (OD 405). OD measured at 620 nm was subtracted from values at 405 nm for each sample.

C. ELISA Tri-S

A codon-optimized nucleotide fragment encoding a stabilized version of the SARS-CoV-2 S ectodomain (amino acid 1 to 1208) followed by a foldon trimerization motif and tags (8×HisTag, StrepTag, and AviTag) was synthetized and cloned into pcDNA™3.1/Zeo(+) expression vector (Thermo Fisher Scientific). Trimeric S (tri-S) glycoproteins were produced by transient co-transfection of exponentially growing Freestyle™ 293-F suspension cells (Thermo Fisher Scientific, Waltham, MA) using polyethylenimine (PEI)-precipitation method as previously described³⁰. Recombinant tri-S proteins were purified by affinity chromatography using the Ni Sepharose® Excel Resin according to manufacturer's instructions (ThermoFisher Scientific). Protein purity was evaluated by in-gel protein silver-staining using Pierce® Silver Stain kit (ThermoFisher Scientific) following SDS-PAGE in reducing and non-reducing conditions using NuPAGE™ 3-8% Tris-Acetate gels (Life Technologies). High-binding 96-well ELISA plates (Costar, Corning) were coated overnight with 125 ng/well of purified tri-S proteins in PBS. After washings with PBS-0.1% Tween 20 (PBST), plate wells were blocked with PBS-1% Tween 20-5% sucrose-3% milk powder for 2 h. After PBST washings, 1:100-diluted sera in PBST-1% BSA and 7 consecutive 1:4 dilutions were added and incubated 2 h. After PBST washings, plates were incubated with 1,000-fold diluted peroxydase-conjugated goat anti-human IgG/IgM/IgA (Immunology Jackson ImmunoReseach, 0.8 μg/ml final) for 1 h. Plates were revealed by adding 100 μl of HRP chromogenic substrate (ABTS solution, Euromedex) after PBST washings. Optical densities were measured at 405 nm (OD₄₀₅ₙₘ) following a 30 min incubation. Experiments were performed in duplicate at room temperature and using HydroSpeed™ microplate washer and Sunrise™ microplate absorbance reader (Tecan Minnedorf, Switzerland). Area under the curve (AUC) values were determined by plotting the log₁₀ of the dilution factor values (x axis) required to obtain OD₄₀₅ₙₘ values (y axis). AUC calculation and Receiving Operating Characteristics (ROC) analyses were performed using GraphPad Prism software (v8.4.1, GraphPad Prism Inc.).

D. S-Flow Assay

HEK293T (referred as 293T) cells were from ATCC (ATCC® CRL-3216™) and tested negative for Mycoplasma. Cells were split every 2-3 days using DMEM medium supplemented with 10% fetal calf serum and 1% Penicillin streptomycin (complete medium). A codon optimized version of the SARS-Cov-2 S gene (GenBank: QHD43416.1)¹, was transferred into the phCMV backbone (GenBank: AJ318514), by replacing the VSV-G gene. 293T Cells were transfected with S or a control plasmid using Lipofectamine 2000 (Life technologies). One day after, transfected cells were detached using PBS-EDTA and transferred into U-bottom 96-well plates (50,000 cell/well). Cell were incubated at 4° C. for 30 min with sera (1:300 dilution, unless otherwise specified) in PBS containing 0.5% BSA and 2 mM EDTA, washed with PBS, and stained using either anti-IgG AF647 (ThermoFisher) or Anti-IgM (PE by Jackson ImmunoResearch or AF488 by ThermoFisher). Cells were washed with PBS and fixed 10 min using 4% PFA. Data were acquired on an Attune Nxt instrument (Life Technologies). In less than 0.5% of the samples tested, we detected a signal in control 293T cells, likely corresponding to antibodies binding to other human surface antigens. Specific binding was calculated with the formula: 100×(% binding on 293T-S−binding on control cells)/(100−binding on control cells). We generated stably-expressing 293T S cells during completion of this study, which yielded similar results.

A 293T genetically modified cell line, named 293T-S, genetically modified with a pLV-SARS-cov-2 S-Puro vector, was deposited with the Collection Nationale de Cultures de Microorganismes on May 5, 2020, under registration number CNCM I-5509.

A 293T genetically modified cell line, named 293T-CTRL, genetically modified with a pLV-Empty-Puro vector, was deposited with the Collection Nationale de Cultures de Microorganismes on May 5, 2020, under registration number CNCM I-5508.

Representative procedures for the assay are as follows.

Reagents:
293T-S cells (293T cells expressing the Spike protein of SARS-cov-2),
293T-CTLR cells (293T cells expressing a Empty transgene),
Complete medium: DMEM (Gibco)+10% FCS+1% PenStrep (Gibco),
PBS-EDTA: PBS (Gibco)+2 mM EDTA (Sigma),
U-Bottom 96 well plate,
Staining Buffer: PBS (Gibco)+0.5% BSA (Sigma)+2 mM EDTA (Sigma),
PBS (Gibco),
anti-Hu IgG Alexafluor 647 antibody (ref: A21445, Invitrogen), and
PFA 2%: dilution 1:1 of PFA 4% (ref: J61899, Alfa Aesar) and PBS (Gibco).

Step-by-step protocol:
1) prepare the 293T-S and 293T-CTLR cells:
   a) remove the medium from the culture flask,
   b) wash with 10 mL of PBS EDTA,
   c) remove PBS-EDTA and leave the flask in the incubator for 5 min to detach cells,
   d) Recover cells with 5 mL of Complete medium, re-seed 1 mL (in 12 mL final) of cells to maintain the culture and keep 4 mL for the assay, and
   e) Count the cells;
2) seed 50,000 cells per well of a 96 well plate. Each samples must be interrogated using a well of 293T-S and separate well of 293-E cells;
3) Spin 2 min@2000 rpm;
4) Remove the supernatant using a multi-channel;
5) Incubate 30 min at 4° C. with 50 uL of serum diluted 1:300 in Staining Buffer;
6) add 150 uL of PBS;
7) Spin 2 min@2000 rpm;
8) Remove the supernatant using a multi-channel;
9) Incubate 30 min at 4° C. with 35 uL of anti-Hu AlexaFluor 647 antibody diluted 1:600 in Staining Buffer;
10) add 150 uL of PBS;
11) Spin 2 min@2000 rpm;
12) Remove the supernatant using a multi-channel;
13) add 40 uL of PFA 2% and incubate 15 min at room temperature;
14) add 120 uL of PBS;
15) acquired 90 uL using a cytometer within 24 h;
16) To analyze the data, normalized the % or S positive cells observed in the 293-S using the frequency of % or S positive cells observed in the 293-E using the formula:

(((% of S+293T-S)−(% of S+293T-CTLR))/(100−(% of S+293T-CTLR)))*100.

E. LIPS Assay

Ten recombinant antigens were designed based on the viral genome sequence of the SARS-CoV-2 strain France/IDF0372/2020 (accession no EPI_ISL_406596) obtained from GISAID database³¹. Five targeted different domains of S: Full S1 sub-unit (residues 1-698), N-terminal domain of S1 (S1-NTD, residues 1-305), domain connecting the S1-NTD to the RBD (S1-CD, residues 307-330 and 529-700 connected by a GGGSGG linker), Full S2 sub-unit (residues 686-1208), and S441-685. For constructs that did not contain an endogenous signal peptide (residues 1-14) i.e. S1-CD and S2 constructs, an exogenous signal peptide coming from a human kappa light chain (METDTLLLWVLLLWVPGSTG) was added to ensure efficient protein secretion into the media. Five additional recombinant antigens, targeting overlapping domains of N, were designed: Full N (residues 1-419), N-terminal domain (residues 1-209), C-terminal domain (residues 233-419), N120-419 and N111-419. The LIPS assay was designed as described³² with minor modifications. Expression vectors were synthesized by GenScript Company, using as backbone the pcDNA3.1(+) plasmid, with codon usage optimized for human cells. HEK-293F cells were grown in suspension and transfected with PolyEthylenImine (PEI-25 kDa, Polyscience Inc., USA). Valproic acid (2.2 mM) was added at day 1 to boost expression. Recombinant proteins were harvested at day 3 in supernatants or crude cell lysates. Luciferase activity was quantified with a Centro XS³ LB 960 luminometer (Berthold Technologies, France). 10⁸ LU of antigens were engaged per reaction. S1 and C-terminal domain (residues 233-419) were selected for analysing the cohorts. To increase sensitivity, the cohorts were tested at a final dilution of 1:10 of sera.

F. Production of Lenti S Pseutotypes

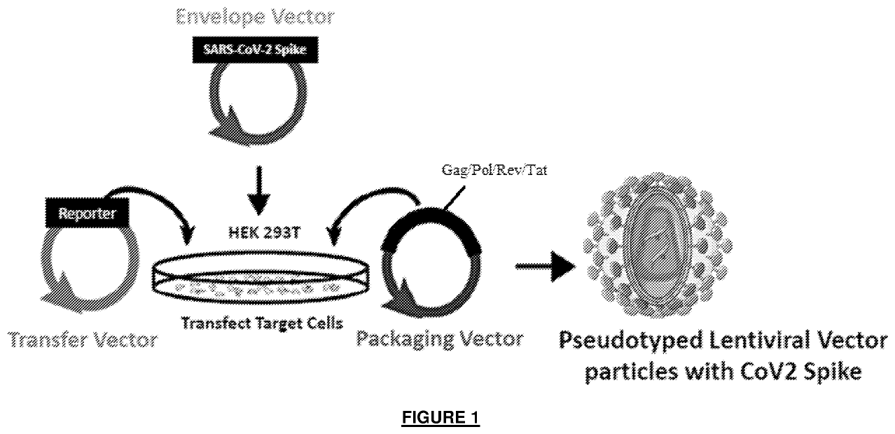

Pseudotyped viruses were produced by transfection of 293T cells as previously described 33. Briefly, cells were co-transfected with a packaging plasmid encoding for lentiviral proteins, a GFP reporter (or luciferase or NanoLuc), and a plasmid expressing the spike protein (S or Spike) under its wild type form, or the VSV-G plasmid as a control. Pseudotyped virions were harvested at days 2-3 post-transfection. Production efficacy was assessed by measuring infectivity or p24 concentration. (FIG. 1.) These pseudoviruses are non-infectious and can be manipulated under a BSL2 confinement.

G. Seroneutralization Test

1. Pseudo-Virus Production and Permissive Cell Line Generation

Pseudo-typed vectors were produced and titrated as previously described. (Iglesias M C, Mollier K, Beignon A S, et al. Lentiviral vectors encoding HIV-1 polyepitopes induce broad CTL responses in vivo. Mol Ther 2007; 15:1203-10.) Adaptation of the protocol are: cells were co-transfected with calcium-phosphate precipitation protocol with 10 μg of packaging plasmid encoding for gag-pol-tat-rev proteins (p8.74), 10 μg of vector plasmid (pTrip-CMV-lucF-Wm) expressing luciferase Firefly reporter and 5 μg of envelop plasmid expressing a codon-optimized full-length S SARS-Cov-2 (UniProtKB ID: PODTC2) sequence amplified by PCR from pMK-RQ_S-2019-nCoV with adaptative primers and introduced by BamHI/XhoI restriction/ligation in a pCMV plasmid. Pseudo-typed vectors were harvested at day 2 post-transfection. Functional titer (TU) was determined by SYBRgreen qPCR after transduction of a stable HEK 293T-hACE2 cell line using two couples of primers to either quantify vector genome (forward CMV: ACT GCC AAA ACC GCA TCA CC reverse CMV: AAT GAC GGT AAA TGG CCC GC) or cell genome (forward GADPH: TCT CCT CTG ACT TCA ACA GC reverse GADPH: CCC TGC ACT TTT TAA GAG CC). To generate the permissive cell line 293T::hACE2, HEK 293T cells were transduced at MOI 20 with an integrative lentiviral vector expressing cDNA human ACE2 (UniProtKB ID: Q9BYF1) codon-optimized gene (Eurofins) under the control of human UBC promoter. Clones were generated by limiting dilution and selected for their permissivity to SARS-CoV-2 S pseudo-typed lentiviral vector transduction with a Luciferase Assay System (Promega).

2. Pseudo-Neutralization Assay Protocol

First, sera were decomplemented at 56° C. during 30 min in a water bath. To determine EC50 serum dilutions (from 1/40 to 1/40960 by successive 4-fold dilutions) are mixed and co-incubated with 300 TU of pseudo-typed vector at room temperature during 30 minutes under agitation. Both, serum and vector are diluted in culture medium DMEM-glutamax (Gibco)+10% FCS (Gibco)+Pen/Strep (Gibco). Mix is then plated in tissue culture treated black 96-well plate clear bottom (Costar) with 20 000 293T::hACE2 cells in suspension. To prepare the cell suspension, the cell flask is washed with DPBS twice (Gibco) and cells are individualized with DPBS+0.1% EDTA (Sigma-Aldrich) to preserve hACE2 protein. After 48 h incubation at 37° C. 5% CO₂, the medium is completely removed by aspiration and bioluminescence is measured using a Luciferase Assay System (Promega) on an EnSpire plate reader (PerkinElmer).

3. Pseudo-Neutralization Analysis

Threshold determination on different serum collections. To setup up the experimentation, Min-max values are determined on untransduced cells and prepandemic serum (dilution 1/40) respectively, Covid-19 patients are used as positive controls. To define positiveness threshold with a confidence index >99%, this value is set at Mean (prepandemic)−3 Standard deviation. During sample analysis, all samples are firstly evaluated for positiveness at dilution 1/40. If the value is below threshold, then ID50 is determined as described below in a second experiment.

Dilution curves and ID50 determination. First, raw datas are transformed into percentage of neutralization. This percentage is determined according to mean of prepandemic serums (0%) and Untransduced cells (100%) values. Second, a non-linear regression is performed to determine the theorical dilution that give a 50% inhibition (ID50). Detection limit was set at 40 considering the maximum reached value by prepandemic samples.

4. Vectors

pTRIPΔU3.hUBC-hACE2

FIG. 27 presents a Schematic representation of pTRIPΔU3.hUBC-hACE2

Nucleotide Sequence of BamHI-XhoI DNA Insert (hACE2) (SEQ ID NO: 5)

GGATCCGCCACCATGTCAAGCTCTTCCTGGCTCCTTCTCA
GCCTTGTTGCTGTAACTGCTGCTCAGTCCACCATTGAGGA
ACAGGCCAAGACATTTTTGGACAAGTTTAACCACGAAGCC
GAAGACCTGTTCTATCAAAGTTCACTTGCTTCTTGGAATT
ATAACACCAATATTACTGAAGAGAATGTCCAAAACATGAA
TAATGCTGGGGACAAATGGTCTGCCTTTTTAAAGGAACAG
TCCACACTTGCCCAAATGTATCCACTACAAGAAATTCAGA
ATCTCACAGTCAAGCTTCAGCTGCAGGCTCTTCAGCAAAA
TGGGTCTTCAGTGCTCTCAGAAGACAAGAGCAAACGGTTG
AACACAATTCTAAATACAATGAGCACCATCTACAGTACTG
GAAAAGTTTGTAACCCAGATAATCCACAAGAATGCTTATT
ACTTGAACCAGGTTTGAATGAAATAATGGCAAACAGTTTA
GACTACAATGAGAGGCTCTGGGCTTGGGAAAGCTGGAGAT
CTGAGGTCGGCAAGCAGCTGAGGCCATTATATGAAGAGTA
TGTGGTCTTGAAAAATGAGATGGCAAGAGCAAATCATTAT
GAGGACTATGGGGATTATTGGAGAGGAGACTATGAAGTAA
ATGGGGTAGATGGCTATGACTACAGCCGCGGCCAGTTGAT
TGAAGATGTGGAACATACCTTTGAAGAGATTAAACCATTA
TATGAACATCTTCATGCCTATGTGAGGGCAAAGTTGATGA
ATGCCTATCCTTCCTATATCAGTCCAATTGGATGCCTCCC
TGCTCATTTGCTTGGTGATATGTGGGGTAGATTTTGGACA
AATCTGTACTCTTTGACAGTTCCCTTTGGACAGAAACCAA
ACATAGATGTTACTGATGCAATGGTGGACCAGGCCTGGGA
TGCACAGAGAATATTCAAGGAGGCCGAGAAGTTCTTTGTA
TCTGTTGGTCTTCCTAATATGACTCAAGGATTCTGGGAAA
ATTCCATGCTAACGGACCCAGGAAATGTTCAGAAAGCAGT
CTGCCATCCCACAGCTTGGGACCTGGGGAAGGGCGACTTC
AGGATCCTTATGTGCACAAAGGTGACAATGGACGACTTCC
TGACAGCTCATCATGAGATGGGGCATATCCAGTATGATAT
GGCATATGCTGCACAACCTTTTCTGCTAAGAAATGGAGCT
AATGAAGGATTCCATGAAGCTGTTGGGGAAATCATGTCAC
TTTCTGCAGCCACACCTAAGCATTTAAAATCCATTGGTCT
TCTGTCACCCGATTTTCAAGAAGACAATGAAACAGAAATA
AACTTCCTGCTCAAACAAGCACTCACGATTGTTGGGACTC
TGCCATTTACTTACATGTTAGAGAAGTGGAGGTGGATGGT
CTTTAAAGGGGAAATTCCCAAAGACCAGTGGATGAAAAAG
TGGTGGGAGATGAAGCGAGAGATAGTTGGGGTGGTGGAAC
CTGTGCCCCATGATGAAACATACTGTGACCCCGCATCTCT
GTTCCATGTTTCTAATGATTACTCATTCATTCGATATTAC
ACAAGGACCCTTTACCAATTCCAGTTTCAAGAAGCACTTT
GTCAAGCAGCTAAACATGAAGGCCCTCTGCACAAATGTGA
CATCTCAAACTCTACAGAAGCTGGACAGAAACTGTTCAAT
ATGCTGAGGCTTGGAAAATCAGAACCCTGGACCCTAGCAT
TGGAAAATGTTGTAGGAGCAAAGAACATGAATGTAAGGCC
ACTGCTCAACTACTTTGAGCCCTTATTTACCTGGCTGAAA
GACCAGAACAAGAATTCTTTTGTGGGATGGAGTACCGACT
GGAGTCCATATGCAGACCAAAGCATCAAAGTGAGGATAAG
CCTAAAATCAGCTCTTGGAGATAAAGCATATGAATGGAAC
GACAATGAAATGTACCTGTTCCGATCATCTGTTGCATATG
CTATGAGGCAGTACTTTTTAAAAGTAAAAAATCAGATGAT
TCTTTTTGGGGAGGAGGATGTGCGAGTGGCTAATTTGAAA
CCAAGAATCTCCTTTAATTTCTTTGTCACTGCACCTAAAA
ATGTGTCTGATATCATTCCTAGAACTGAAGTTGAAAAGGC
CATCAGGATGTCCCGGAGCCGTATCAATGATGCTTTCCGT
CTGAATGACAACAGCCTAGAGTTTCTGGGGATACAGCCAA
CACTTGGACCTCCTAACCAGCCCCCTGTTTCCATATGGCT
GATTGTTTTTGGAGTTGTGATGGGAGTGATAGTGGTTGGC
ATTGTCATCCTGATCTTCACTGGGATCAGAGATCGGAAGA
AGAAAAATAAAGCAAGAAGTGGAGAAAATCCTTATGCCTC
CATCGATATTAGCAAAGGAGAAAATAATCCAGGATTCCAA
AACACTGATGATGTTCAGACCTCCTTTTAACTCGAG

pTRIPΔU3.CMV-LucF-WPREm

FIG. 28 presents a Schematic representation of pTRIPΔU3.CMV-LucF-WPREm

### Nucleotide Sequence of BamHI-XhoI DNA Insert (Luciferase FireFly) (SEQ ID NO: 6)

GGATCCGCCACCATGGAAGACGCCAAAAACATAAAGAAAG
GCCCGGCGCCATTCTATCCGCTGGAAGATGGAACCGCTGG
AGAGCAACTGCATAAGGCTATGAAGAGATACGCCCTGGTT
CCTGGAACAATTGCTTTTACAGATGCACATATCGAGGTGG
ACATCACTTACGCTGAGTACTTCGAAATGTCCGTTCGGTT
GGCAGAAGCTATGAAACGATATGGGCTGAATACAAATCAC
AGAATCGTCGTATGCAGTGAAAACTCTCTTCAATTCTTTA
TGCCGGTGTTGGGCGCGTTATTTATCGGAGTTGCAGTTGC
GCCCGCGAACGACATTTATAATGAACGTGAATTGCTCAAC
AGTATGGGCATTTCGCAGCCTACCGTGGTGTTCGTTTCCA
AAAAGGGGTTGCAAAAAATTTTGAACGTGCAAAAAAAGCT
CCCAATCATCCAAAAAATTATTATCATGGATTCTAAAACG
GATTACCAGGGATTTCAGTCGATGTACACGTTCGTCACAT
CTCATCTACCTCCCGGTTTTAATGAATACGATTTTGTGCC
AGAGTCCTTCGATAGGGACAAGACAATTGCACTGATCATG
AACTCCTCTGGATCTACTGGTCTGCCTAAAGGTGTCGCTC
TGCCTCATAGAACTGCCTGCGTGAGATTCTCGCATGCCAG
AGATCCTATTTTTGGCAATCAAATCATTCCGGATACTGCG
ATTTTAAGTGTTGTTCCATTCCATCACGGTTTTGGAATGT
TTACTACACTCGGATATTTGATATGTGGATTTCGAGTCGT
CTTAATGTATAGATTTGAAGAAGAGCTGTTTCTGAGGAGC
CTTCAGGATTACAAGATTCAAAGTGCGCTGCTGGTGCCAA
CCCTATTCTCCTTCTTCGCCAAAAGCACTCTGATTGACAA
ATACGATTTATCTAATTTACACGAAATTGCTTCTGGTGGC
GCTCCCCTCTCTAAGGAAGTCGGGGAAGCGGTTGCCAAGA
GGTTCCATCTGCCAGGTATCAGGCAAGGATATGGGCTCAC
TGAGACTACATCAGCTATTCTGATTACACCCGAGGGGGAT
GATAAACCGGGCGCGGTCGGTAAAGTTGTTCCATTTTTTG
AAGCGAAGGTTGTGGATCTGGATACCGGGAAAACGCTGGG
CGTTAATCAAAGAGGCGAACTGTGTGTGAGAGGTCCTATG
ATTATGTCCGGTTATGTAAACAATCCGGAAGCGACCAACG
CCTTGATTGACAAGGATGGATGGCTACATTCTGGAGACAT
AGCTTACTGGGACGAAGACGAACACTTCTTCATCGTTGAC
CGCCTGAAGTCTCTGATTAAGTACAAAGGCTATCAGGTGG
CTCCCGCTGAATTGGAATCCATCTTGCTCCAACACCCCAA
CATCTTCGACGCAGGTGTCGCAGGTCTTCCCGACGATGAC
GCCGGTGAACTTCCCGCCGCCGTTGTTGTTTTGGAGCACG
GAAAGACGATGACGGAAAAAGAGATCGTGGATTACGTCGC
CAGTCAAGTAACAACCGCGAAAAAGTTGCGCGGAGGAGTT
GTGTTTGTGGACGAAGTACCGAAAGGTCTTACCGGAAAAC
TCGACGCAAGAAAAATCAGAGAGATCCTCATAAAGGCCAA
GAAGGGCGGAAAGATCGCCGTGTAATGCTCGAG

Nucleotide sequence of XhoI-KpbI DNA Insert (WPREm) (SEQ ID NO: 7)

CTCGAGAATTCCCGATAATCAACCTCTGGATTACAAAATT
TGTGAAAGATTGACTGGTATTCTTAACTATGTTGCTCCTT
TTACGCTATGTGGATACGCTGCTTTAATGCCTTTGTATCA
TGCTATTGCTTCCCGTATGGCTTTCATTTTCTCCTCCTTG
TATAAATCCTGGTTGCTGTCTCTTTATGAGGAGTTGTGGC
CCGTTGTCAGGCAACGTGGCGTGGTGTGCACTGTGTTTGC
TGACGCAACCCCCACTGGTTGGGGCATTGCCACCACCTGT
CAGCTCCTTTCCGGGACTTTCGCTTTCCCCCTCCCTATTG
CCACGGCGGAACTCATCGCCGCCTGCCTTGCCCGCTGCTG
GACAGGGGCTCGGCTGTTGGGCACTGACAATTCCGTGGTG
TTGTCGGGGAAGCTGACGTCCTTTCCGCGGCTGCTCGCCT
GTGTTGCCACCTGGATTCTGCGCGGGACGTCCTTCTGCTA
CGTCCCTTCGGCCCTCAATCCAGCGGACCTTCCTTCCCGC
GGCCTGCTGCCGGCTCTGCGGCCTCTTCCGCGTCTTCGCC
TTCGCCCTCAGACGAGTCGGATCTCCCTTTGGGCCGCCTC
CCCGCATCGGGGGTACC

pCMV-SSARS-COV2

FIG. 29 presents a Schematic representation of pCMV.S High

Nucleotide sequence of BamHI-XHoI DNA Insert (Spike S High) (SEQ ID NO: 8)

CGATCCCGTACGGCCACCATGTTCGTGTTTCTGGTGCTGC
TGCCACTGGTGTCCAGTCAGTGCGTGAACCTGACCACACG
AACACAGCTGCCACCAGCCTACACCAATAGCTTCACCCGC
GGAGTGTACTACCCCGACAAGGTGTTCCGCAGCAGCGTGC
TGCATAGCACCCAGGATCTGTTTCTGCCCTTCTTCAGCAA
CGTGACCTGGTTCCACGCCATCCACGTGTCCGGCACCAAT
GGCACCAAGCGCTTCGATAATCCCGTGCTGCCCTTCAACG
ATGGCGTGTACTTTGCCAGCACCGAGAAGTCCAATATCAT
CCGCGGCTGGATCTTCGGCACCACACTGGATAGCAAGACC
CAGAGCCTGCTGATCGTGAACAACGCCACCAACGTGGTCA
TCAAAGTGTGCGAGTTCCAGTTCTGCAACGACCCCTTCCT
GGGCGTCTACTACCACAAGAACAACAAGAGCTGGATGGAA
AGCGAGTTCCGCGTGTACAGCAGCGCCAACAACTGCACCT
TCGAGTACGTGTCCCAGCCATTCCTGATGGACCTGGAAGG
CAAGCAGGGCAACTTCAAGAACCTGCGCGAGTTCGTGTTC
AAGAACATCGACGGCTACTTCAAAATCTACAGCAAGCACA
CCCCAATCAACCTCGTGCGCGATCTGCCACAGGGATTCAG
TGCTCTGGAACCCCTGGTGGATCTGCCCATCGGCATCAAC
ATTACCCGCTTTCAGACACTGCTGGCCCTGCACCGCAGTT
ACTTGACACCAGGCGATAGCAGCAGTGGATGGACAGCTGG
TGCCGCCGCTTACTACGTTGGATATCTGCAGCCACGCACC
TTTCTGCTGAAGTACAACGAGAACGGCACCATCACCGACG
CCGTGGATTGTGCTCTCGATCCCCTGAGCGAGACAAAGTG
CACCCTGAAGTCCTTCACCGTCGAGAAGGGCATCTACCAG
ACCAGCAATTTCCGCGTGCAGCCCACCGAGAGCATCGTGC
GCTTCCCCAATATCACCAATCTGTGCCCCTTCGGCGAGGT
GTTCAATGCCACACGCTTTGCCTCCGTGTACGCCTGGAAT
CGCAAGCGCATTAGCAACTGCGTGGCCGACTACTCCGTGC
TGTACAATAGCGCCAGCTTCAGCACCTTCAAGTGCTACGG
CGTGTCACCCACCAAGCTGAACGACCTGTGCTTCACCAAT
GTGTACGCCGACAGCTTCGTGATCCGCGGAGATGAAGTGC
GACAGATTGCCCCAGGCCAGACCGGCAAGATCGCCGACTA
CAATTACAAGCTGCCCGACGACTTCACCGGCTGCGTGATC
GCCTGGAACAGCAACAACCTGGATTCCAAAGTCGGCGGCA
ACTACAACTACCTGTACCGCCTGTTCCGCAAGAGCAATCT
GAAGCCCTTCGAGCGCGACATCAGCACCGAAATCTACCAG
GCCGGAAGCACCCCATGCAACGGCGTGGAAGGCTTCAACT
GCTACTTCCCACTGCAGTCCTACGGATTTCAGCCCACAAA
TGGCGTGGGCTACCAGCCATATCGAGTGGTGGTGCTGAGC
TTCGAACTGCTGCATGCTCCAGCTACCGTGTGCGGCCCCA
AGAAGAGTACCAACCTGGTCAAGAACAAATGCGTGAACTT
CAACTTCAACGGCCTGACCGGAACCGGCGTGCTGACCGAG
AGTAACAAGAAGTTCCTGCCATTCCAGCAGTTTGGCCGCG
ACATTGCCGATACAACCGATGCCGTTCGCGATCCCCAGAC
CTTGGAGATCCTGGATATTACCCCATGCTCCTTCGGCGGC
GTGTCCGTGATTACACCAGGCACCAATACCAGCAACCAGG
TGGCCGTTCTGTACCAGGATGTGAATTGCACAGAGGTGCC
CGTGGCCATTCACGCCGATCAATTGACACCAACATGGCGC
GTGTACTCCACCGGCAGCAATGTGTTTCAAACCCGCGCTG
GATGCCTGATTGGAGCCGAGCACGTGAACAATAGCTACGA
GTGCGATATCCCCATCGGAGCCGGAATCTGCGCCTCCTAT
CAGACCCAGACCAATAGTCCACGACGAGCCCGAAGTGTGG
CCAGCCAGAGCATCATTGCCTATACCATGAGCCTGGGCGC
CGAGAATAGCGTGGCCTACTCCAACAACAGCATTGCTATC
CCCACCAACTTCACCATCAGCGTGACCACCGAGATCCTGC
CAGTGTCCATGACCAAGACCAGCGTGGACTGCACCATGTA
CATCTGCGGAGATAGCACCGAGTGCAGCAACCTGCTGCTG
CAGTACGGAAGTTTCTGCACCCAGCTGAATCGCGCCCTGA
CAGGCATTGCCGTGGAACAGGATAAGAACACCCAAGAGGT
GTTCGCCCAAGTGAAGCAAATCTACAAGACCCCACCAATC
AAGGATTTCGGCGGCTTCAATTTCAGCCAGATTCTGCCCG
ATCCAAGCAAGCCCAGCAAGCGCAGCTTCATCGAGGACCT
GCTGTTCAACAAAGTGACACTGGCCGACGCCGGATTCATC
AAGCAGTATGGCGATTGCCTGGGCGATATTGCCGCACGCG
ATCTGATTTGCGCCCAGAAGTTTAACGGACTGACCGTCCT
GCCACCACTGCTGACAGATGAGATGATCGCCCAGTACACA
AGTGCCCTGCTGGCCGGAACCATTACCAGCGGATGGACAT
TTGGAGCCGGTGCCGCTCTGCAGATTCCCTTCGCTATGCA
GATGGCCTACCGCTTCAATGGCATTGGCGTGACCCAGAAT
GTGCTGTACGAGAACCAGAAGCTGATCGCCAACCAGTTCA
ACAGCGCCATCGGCAAGATTCAGGACAGCCTGAGTAGTAC
CGCCAGCGCTCTGGGAAAGCTGCAGGATGTGGTCAACCAG
AACGCTCAGGCCCTGAACACCCTGGTTAAGCAGCTGAGCA
GCAACTTCGGCGCCATCAGTAGCGTGCTGAACGATATCCT
GAGCCGCCTGGATAAGGTGGAAGCCGAGGTGCAGATCGAT
CGCCTGATTACCGGACGCCTGCAGTCCCTGCAGACCTATG
TGACACAGCAGCTGATCCGAGCCGCCGAGATTCGAGCTAG
TGCTAATCTGGCCGCCACCAAGATGAGCGAATGTGTGCTG
GGACAGAGCAAGCGCGTGGACTTTTGCGGCAAGGGATACC
ACCTGATGAGCTTCCCACAGAGTGCTCCACACGGCGTGGT
GTTTCTGCATGTGACCTACGTGCCCGCTCAAGAGAAGAAT
TTCACCACCGCTCCAGCCATCTGCCACGACGGAAAGGCCC
ATTTTCCACGCGAGGGCGTGTTCGTTAGCAACGGCACTCA
TTGGTTCGTCACCCAGCGCAACTTCTACGAGCCCCAGATC
ATCACCACCGACAACACCTTCGTCAGCGGCAACTGCGACG
TCGTGATCGGCATTGTGAACAACACCGTGTACGATCCACT
GCAGCCCGAGCTGGACAGCTTCAAAGAGGAACTGGACAAG
TACTTTAAGAACCACACAAGCCCCGACGTGGACCTGGGAG
ACATTAGCGGAATCAACGCCAGCGTGGTCAACATCCAGAA
AGAGATTGACCGCCTGAACGAGGTGGCCAAGAATCTGAAC
GAGAGCCTGATCGACCTGCAAGAACTGGGCAAATACGAGC
AGTACATTAAGTGGCCCTGGTACATCTGGCTGGGCTTCAT
TGCCGGACTGATTGCCATCGTGATGGTCACCATTATGCTG
TGCTGCATGACCAGTTGCTGCAGCTGCCTGAAGGGATGCT
GCAGTTGCGGAAGCTGCTGCAAGTTCGACGAGGATGATAG
CGAGCCAGTGCTGAAGGGCGTCAAGCTGCACTACACCTGA
TAACGAGCGCGCCTCGAG

### Nucleotide Sequence of Kanamycin (SEQ ID NO: 9)

ATGATTGAACAAGATGGATTGCACGCAGGTTCTCCGGCCG
CTTGGGTGGAGAGGCTATTCGGCTATGACTGGGCACAACA
GACAATCGGCTGCTCTGATGCCGCCGTGTTCCGGCTGTCA
GCGCAGGGGCGCCCGGTTCTTTTTGTCAAGACCGACCTGT
CCGGTGCCCTGAATGAACTGCAAGACGAGGCAGCGCGGCT
ATCGTGGCTGGCCACGACGGGCGTTCCTTGCGCAGCTGTG
CTCGACGTTGTCACTGAAGCGGGAAGGGACTGGCTGCTAT
TGGGCGAAGTGCCGGGGCAGGATCTCCTGTCATCTCACCT
TGCTCCTGCCGAGAAAGTATCCATCATGGCTGATGCAATG
CGGCGGCTGCATACGCTTGATCCGGCTACCTGCCCATTCG
ACCACCAAGCGAAACATCGCATCGAGCGAGCACGTACTCG
GATGGAAGCCGGTCTTGTCGATCAGGATGATCTGGACGAA
GAGCATCAGGGGCTCGCGCCAGCCGAACTGTTCGCCAGGC
TCAAGGCGAGCATGCCCGACGGCGAGGATCTCGTCGTGAC
CCATGGCGATGCCTGCTTGCCGAATATCATGGTGGAAAAT
GGCCGCTTTTCTGGATTCATCGACTGTGGCCGGCTGGGTG
TGGCGGACCGCTATCAGGACATAGCGTTGGCTACCCGTGA
TATTGCTGAAGAGCTTGGCGGCGAATGGGCTGACCGCTTC
CTCGTGCTTTACGGTATCGCCGCTCCCGATTCGCAGCGCA
TCGCCTTCTATCGCCTTCTTGACGAGTTCTTCTGA

5. Deposited Vectors and Cell Lines

Plasmids used to produce lentiviral vector particles pseudotyped with the CoV2 S protein:

pCMV.SSARS-COV2 was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5608; and

pTRIPdeltaU3-CMV-LucF-WPREm was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5607.

The cell line stably expressing hACE2 receptor (293T-hACE2) was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5609.

The lentiviral vector used to obtain this stable cell line (pTRIPdeltaU3.hUBC-hACE2) was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5610.

The cell line stably expressing hACE2 receptor and GFP (HEK 293T_hACE2_eGFP) was deposited at the Collection Nationale de Cultures de Microrganismes (CNCM), located at Institut Pasteur, 25-28 Rue du Docteur Roux 75724 Paris CEDEX 15 FRANCE on Oct. 13, 2020, under Reference Number CNCM I-5611.

Deposit forms and receipts providing additional details regarding the deposited material are submitted herewith.

### Example 2. Description of Additional Serological Tests

Four tests were used to assess the levels of anti-SARS-CoV-2 antibodies in human sera.

ELISAs. The two ELISAs are classical tests, using as target antigens the full-length N protein (ELISA N) or the extracellular domain of S in a trimerized form (ELISA tri-S). The two recombinant antigens were produced in E. coli (N) or in human cells (S).

The ELISA N assay is a classical indirect test for the detection of total immunoglobulins, using plates coated with a purified His-tagged SARS-CoV 2 N protein. Titration curves of sera from 22 COVID-19 patients and 4 pre-pandemic sera initially led to the determination that a dilution of 1:200 was of optimal sensitivity and specificity, and was later used for testing of large cohorts.

The ELISA tri-S, for trimeric S, allows for the detection of IgG antibodies directed against the SARS-CoV-2 Spike. This ELISA uses as antigen a purified, recombinant and tagged form of the S glycoprotein ectodomain, which was stabilized and trimerized using a foldon motif. Serum IgG from pre-epidemic (n=100), pauci-symptomatic (n=209), and hospitalized individuals (n=159) were titrated using serum dilutions ranging from 1:100 to 1:1,638,400. Receiving-operating characteristic analyses using either total area under the curve or single optical density measurements indicated that the 1:400 dilution provides the best sensitivity and specificity values and was therefore used in subsequent analyses. Of note, the tri-S ELISA also permitted the titration of anti-S IgM and IgA antibodies in human sera.

S-Flow. The third assay, termed S-Flow, is based on the recognition of the S protein expressed at the surface of 293T cells (293T-S cells). In-situ expression of S will allow detection of antibodies binding to various conformations and domains of the viral glycoprotein. S was functionally active, as verified by mixing 293T-S cells with target cells expressing ACE2. Large and numerous syncytia were detected, indicating that S binds to its receptor and performs fusion (not shown). In the S-Flow assay 293T-S cells are incubated with dilutions of sera to be tested. Antibody binding is detected by adding a fluorescent secondary antibody (anti-IgG or anti-IgM). The signal is measured by flow-cytometry using an automated 96-well plate holder. The background signal is measured in 293T cells lacking S and subtracted in order to define a specific signal and a cut-off for positivity.

To establish the specificity of the assay, a series of 40 sera collected before 2019, from the Institut Pasteur biobank (ICAReB), was analyzed. All sera were negative, strongly suggesting that antibodies against other coronaviruses circulating in France were not detected. The sensitivity of the assay was measured by assessing the reactivity of sera from Covid-19 patients hospitalized at Hôpital Bichat. Serial dilutions allowed for the determination of a titer, which reached a value of 24,600 and 2,700 for B1 and B2, respectively. Of note, the median fluorescence intensity (MFI) of the signal decreased with the dilution, indicating that MFI, in addition to the % of positive cells, provides a quantitative measurement of the levels of specific antibodies. A single dilution (1:300) was selected to analyze large numbers of samples. Samples from 9 patients (B1-B9) were analyzed. An increase of the IgG response over time, with positivity appearing 6 days after symptoms onset was observed. Serial dilutions indicated that antibody titers raised over time. Similar patterns with the IgM and IgG responses were observed. The absence of an earlier IgM response may be due to the lower sensitivity of the secondary anti-IgM antibodies tested or because of a short delay between the two responses, which has been already observed in COVID-19 patients. Addressing this question will require the analysis of a higher number of individuals. A secondary anti-whole Ig antibody was tested, but it did not prove more sensitive than the anti-IgG. Thus, the different cohorts were tested with the secondary anti-IgG.

LIPS. The fourth assay, termed LIPS (Luciferase Immunoprecipitation Assay) is based on the use of antigens made of viral proteins (or domains) fused to nanoluciferase (nanoluc). The objective was to develop an assay that is able to test large diverse cohorts and evaluate the range of antibody responses against a set of viral proteins or domains. This opens the possibility to select the best antigens for high throughput binding assays. Each antigen is used at the same molar concentration, based on a standardization by luciferase activity of the amount of Ag engaged in each reaction. This allows for easy direct comparison of the Ab responses (amplitude and kinetic) against each antigen. A panel of 10 different S and N-derived antigens were first evaluated with a set of 34 pre-epidemic human sera were along with those of with 6 COVID hospitalized patients. Two patients were sampled at 3 different time points. The strongest signals in COVID patients' sera compared to background of pre-epidemic sera were identified with S1, S2 and N (C-term part) antigens. Additional investigations on a limited panel of sera sampled in pauci-symptomatic patients showed that S2 responses were, regarding the diagnostic sensitivity and quantitative responses, similar to full S responses evaluated by S-Flow. To avoid redundancy, LIPS analysis was focused to N, selecting it for its sensitivity regarding an intracellular viral protein not targeted by NAbs and S1 as it is described as a target of most NAbs. To establish the specificity of the assay, the same series of 40 sera used for S-Flow were analyzed and found all of the sera to be negative. The kinetic of apparition of antibodies in the same longitudinal samples from 5 patients was also analyzed. An increase of response overtime, with positivity appearing 7-10 days after symptoms onset was observed. Of note, the protein A/G beads used for precipitation of the immune complexes do not bind efficiently to IgM or IgA. Protein L, which has a higher affinity binding to IgA, has not yet been tested.

### Example 3. Description of the Groups

Different cohorts were screened to evaluate the performance of the four assays and corresponding antigens. Sera from up to 491 pre-epidemic individuals was used, collected before 2019, to assess the specificity of the tests. Antibody levels in 51 hospitalized COVID-19 patients from Hôpital Bichat (Paris) were used, to determine the sensitivity of the tests and analyze the kinetics of seroconversion. The clinical and virological characteristics of four of these patients have been recently described²⁴. The prevalence of SARS-CoV-2 positive individuals was studied in a cohort of pauci-symptomatic individuals in Crepy-en-Valois, a city of 15,000 inhabitants in Oise. On 24 Feb. 2020, a staff member from a high school in Crepy-en-Valois was admitted to an hospital in Paris with confirmed SARS-CoV-2 infection. On March 3-4, students from the high school, parents of the students, teachers and staff were invited to participate to an epidemiological investigation around this case. 209 blood samples were collected from individuals reporting mild signs compatible with COVID-19 (fever, cough or dyspnea). Finally, 200 sera from blood donors from the Etablissement Français du Sang (EFS) in Lille (France) was texted. The blood samples were donated in two cities, Clermont (10,000 inhabitants) on March 20 and Noyon (13,000 inhabitants) on March 24, each located at about 60 kilometers from Crepy-en-Valois.

### Example 4. Virus Neutralisation Assays: Microneutralisation (MNT) and Pseudovirus Neutralisation

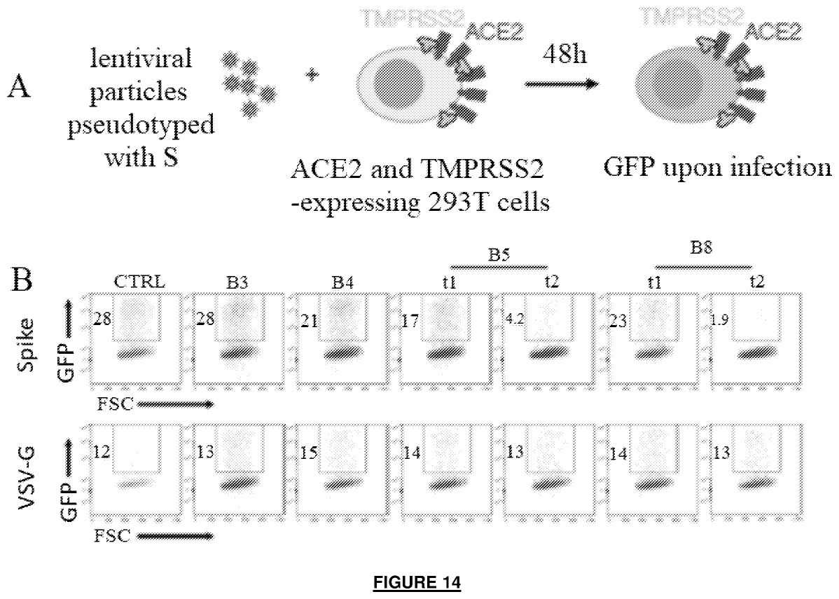

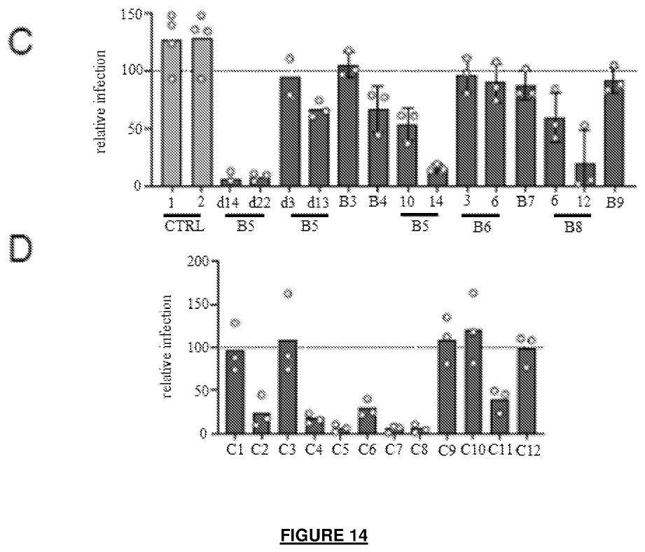

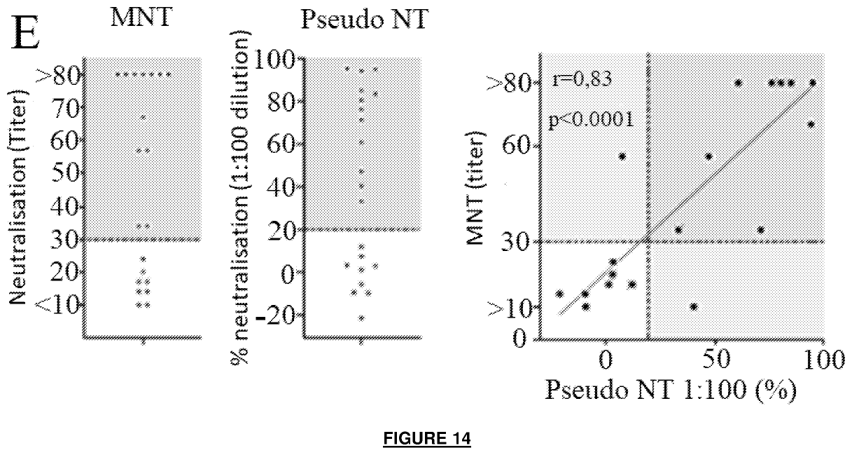

Various tests have already been established to evaluate the presence of NAbs in the sera of infected individuals^{6,8,19,21}. We focused on two new tests. The first is a microneutralisation (MNT) assay using infectious SARS-CoV-2. This reference method is based on virus incubation with serial dilutions of the sera, and evaluation of titers on Vero-E6 cells. We also developed a lentiviral-based pseudotype assay, as outlined FIG. 14A. Lentiviral particles coated with S and encoding for a reporter gene (GFP) are pre-treated with dilutions of the sera to be tested, incubated with target cells (293T cells transiently expressing ACE2 and the TMPRSS2 protease) and the signal was measured after 48 h. A pilot experiment with sera from hospitalized patients demonstrated a strong neutralizing activity with some of the samples (FIGS. 14B and 14C). As a control, we used lentiviral particles coated with an irrelevant viral protein (VSV-G), and they were insensitive to the same sera (FIG. 14C). We also tested as a proof of concept the neutralization activity of the first 12 sera of the cohort of pauci-symptomatic individuals (FIG. 14D). A strong correlation was observed between MNT and neutralization of pseudoviruses (FIG. 14E). Of note, with the pseudovirus assay, similar neutralization results were obtained when target cells transiently transfected with ACE2 and TMPRSS2 were replaced by stable 293T-ACE2 cells, or when luciferase was used as a readout instead of GFP.

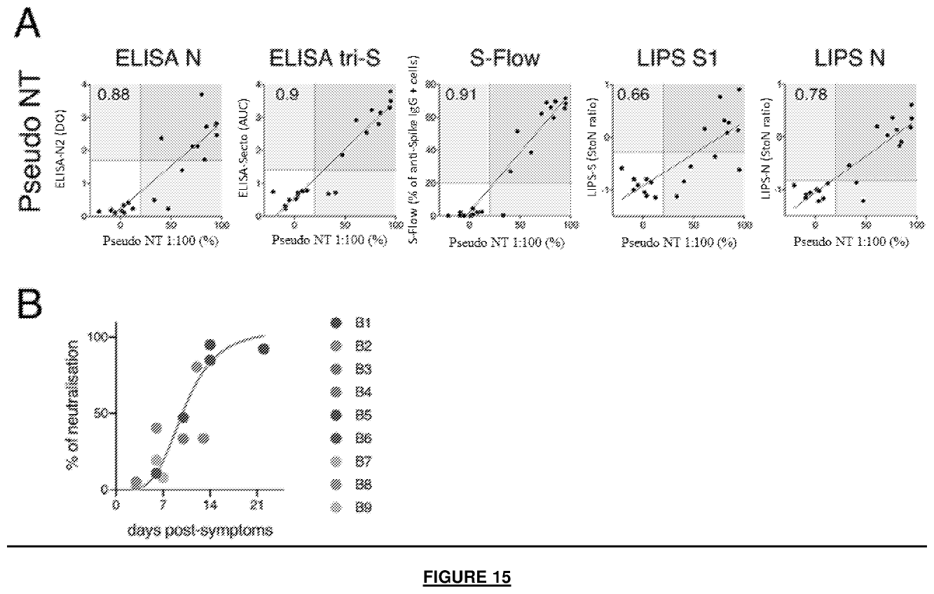

The reference MNT assay is labor-intensive and requires access to a BSL3 facility. We thus performed a pilot correlative analysis between the four serological tests and the pseudovirus assay (FIG. 15A). This analysis was performed with samples from 9 hospitalized patients and 12 pauci-symptomatic individuals. A strong correlation was observed with the ELISA N, ELISA tri-S, S-Flow and LIPS-N, with a similar but less marked trend with the LIPS-S1 assay. We also determined by linear regression the association between the intensity of antibody binding and pseudovirus neutralization. A neutralization activity >80% was associated with the following signals: ELISA N (>2.37), ELISA tri-S (>2.9) S-Flow (>60% of positive cells) and LIPS-N (>0.049). With this level of neutralization, LIPS S1 mainly gave positive responses and a few responses below the cut-off. In 9 hospitalized patients, the neutralization activity increased over time, being detectable at day five and reaching 50% and 80-100% at days 7-14 and 14-21, respectively (FIG. 15B).

### Example 5. Linearity of the Seroneutralization Assay

As demonstrated by the Experiment shown in FIG. 3, the seroneutralization test is specific for cells expressing ACE2, showing a very weak background noise and a linearity of reading over 3 logs according to the type of luciferase used. U.E is an amphotrophic envelope Glycoprotein as control. (VSV-G). Heat inactivated vector demonstrate that signal is specific to vector transduction ability.

### Example 6. Comparison with Alternative Pseudotypes

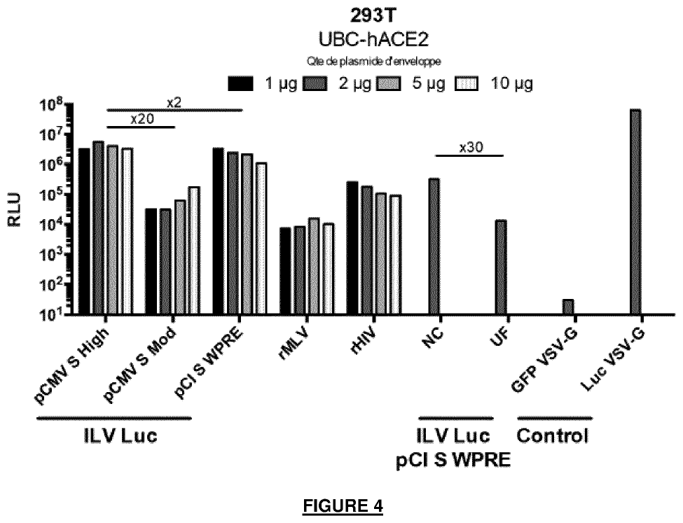

As shown in FIG. 4, the lentiviral vectors used for the production of pseudotypes allow for a better transduction and therefore a better signal than alternative vectors, such as the retroviral vectors derived from MoMLV. Moreover, the signal is stronger because a plasmid of optimized expression is used for the expression of the S protein. NC and UF show functional titer of Non-concentrated Vector and concentrated by Ultrafiltration respectively: LENTI.S pseudotypes are not suitable for ultrafiltration.

### Example 7. Comparison of Different Host Cells

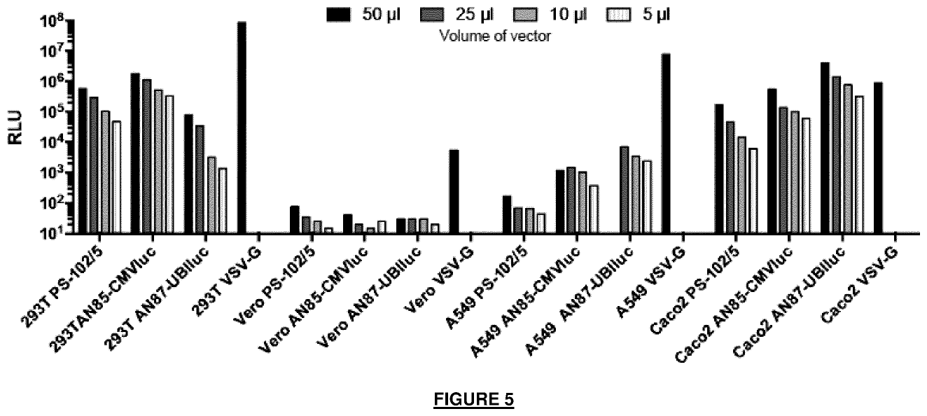

Different target cellular lineages, stably transduced by a vector expressing ACE2 have been tested for their transducibility by LENTI.S pseudotypes. The 293T-ACE2 cells show the best permissiveness. (FIG. 5). CMVluc and UBluc refers to promoter controlling reporter expression (ieCMV and hUBC respectively).

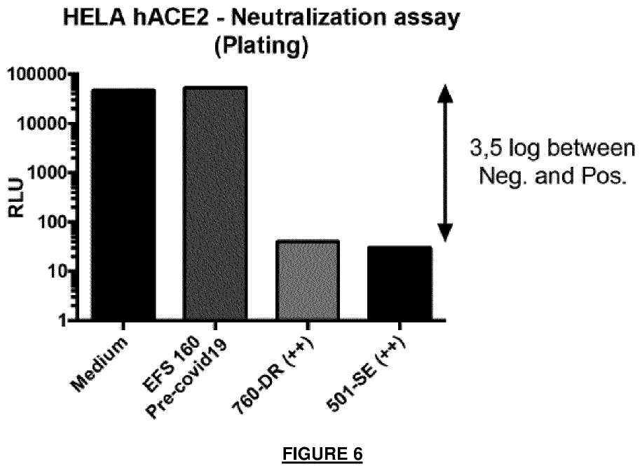

A HeLa-ACE2 line has been tested and shows an equivalent performance to 293. (FIG. 6.) This HeLa-ACE2 lineage may offer certain advantages in the context of an automated test platform.

### Example 8. Analysis of the CORSER Cohort (Crepy en Valois)

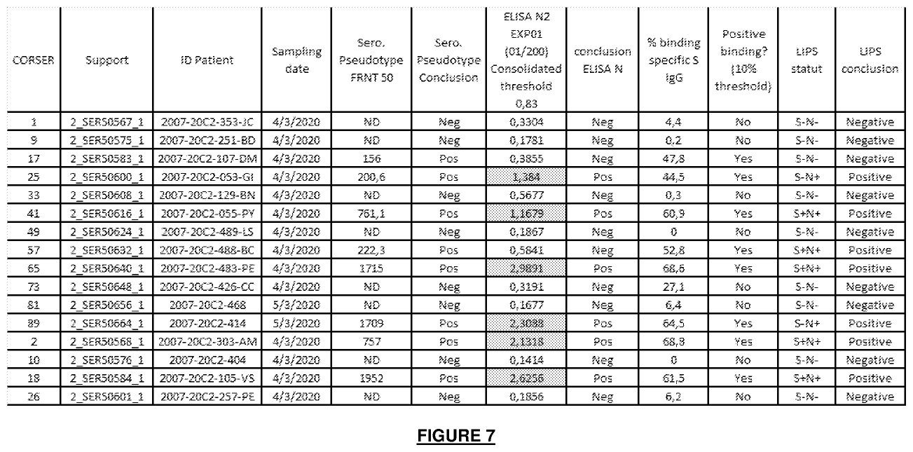

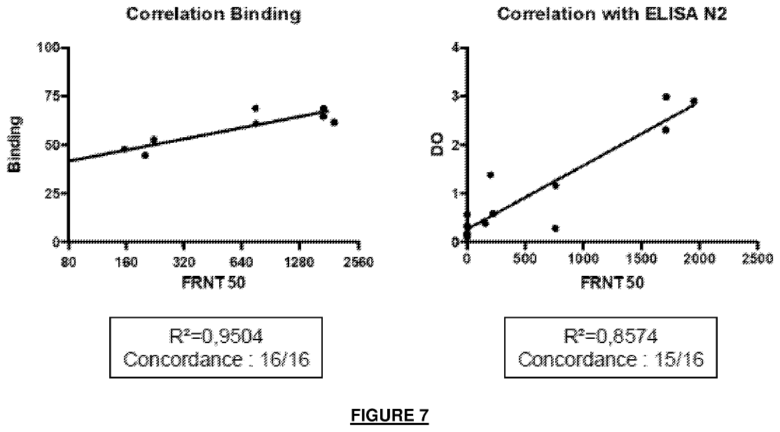

The LENTI.S test was compared to the Binding, ELISA N, and LIPS tests. As shown in FIG. 7, the LENTI.S test shows a perfect correlation with the binding test. The correlation is not absolute with the other tests.

### Example 9. Individual Analysis of the CORSER Cohort

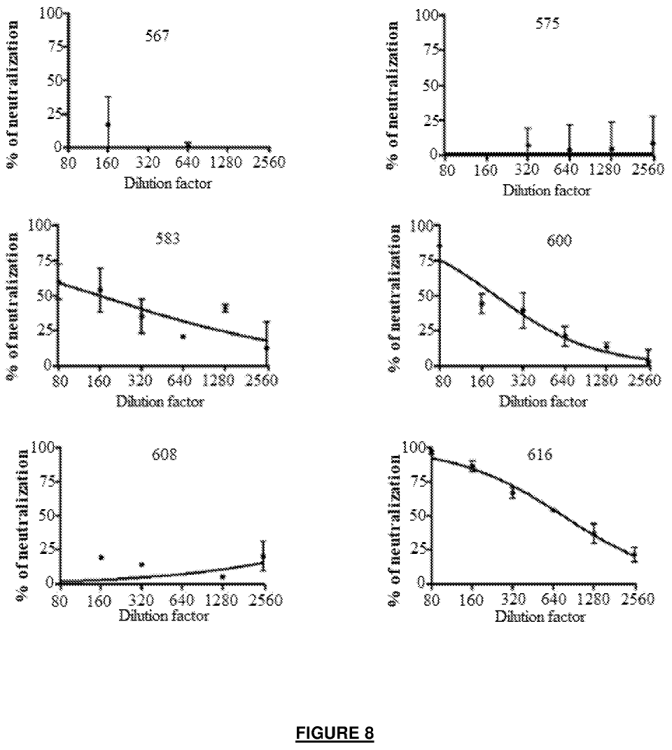

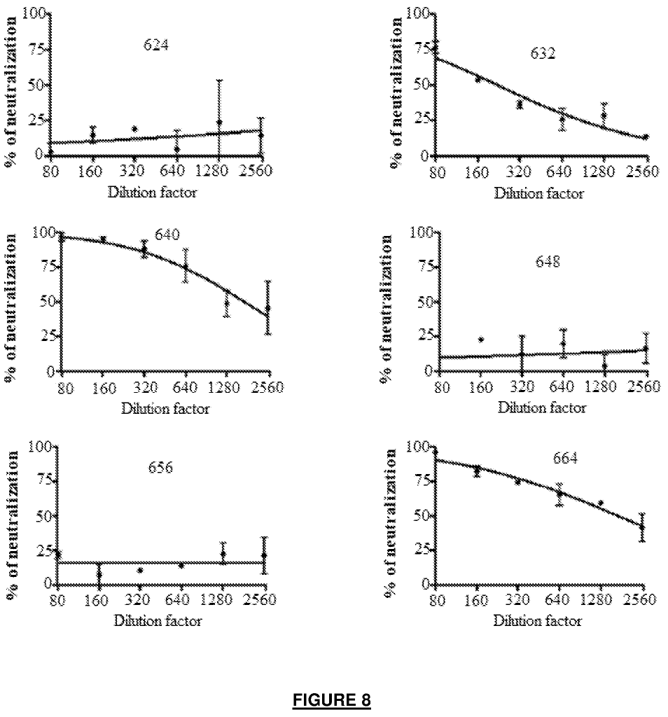

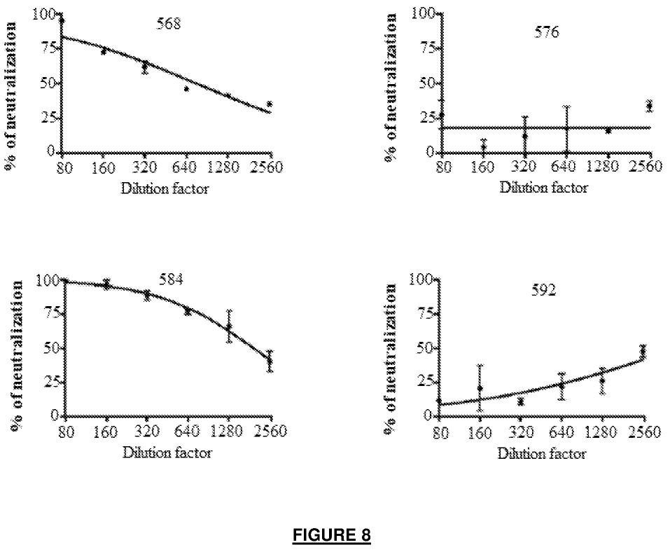

Inhibitory dilution curves for each sample of the CORSER cohort at Crepy en Valois (FIG. 8).

### Example 10. Study of Hospitalized Patients: Bichat 1 and 2

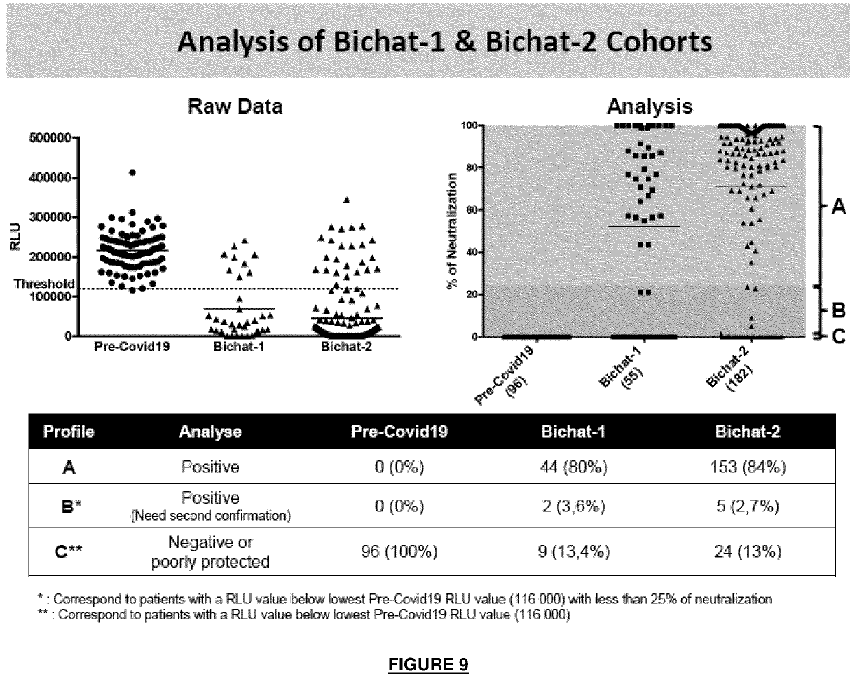

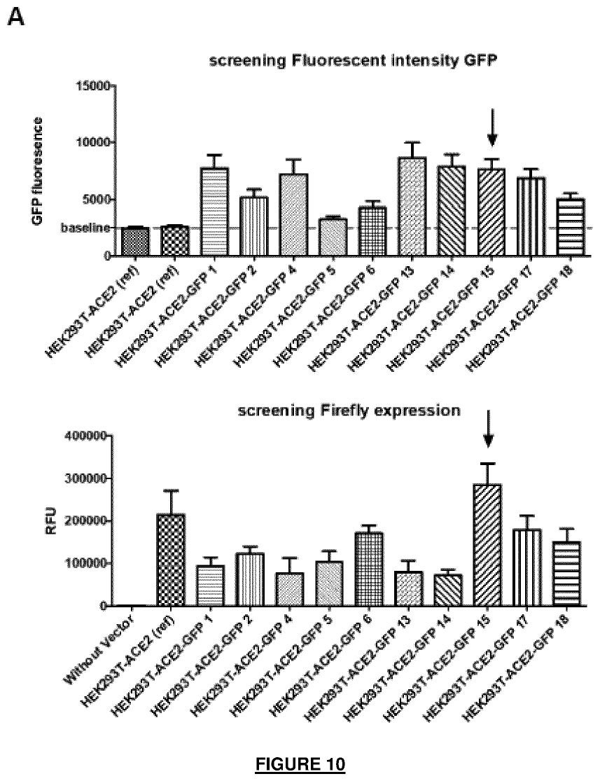

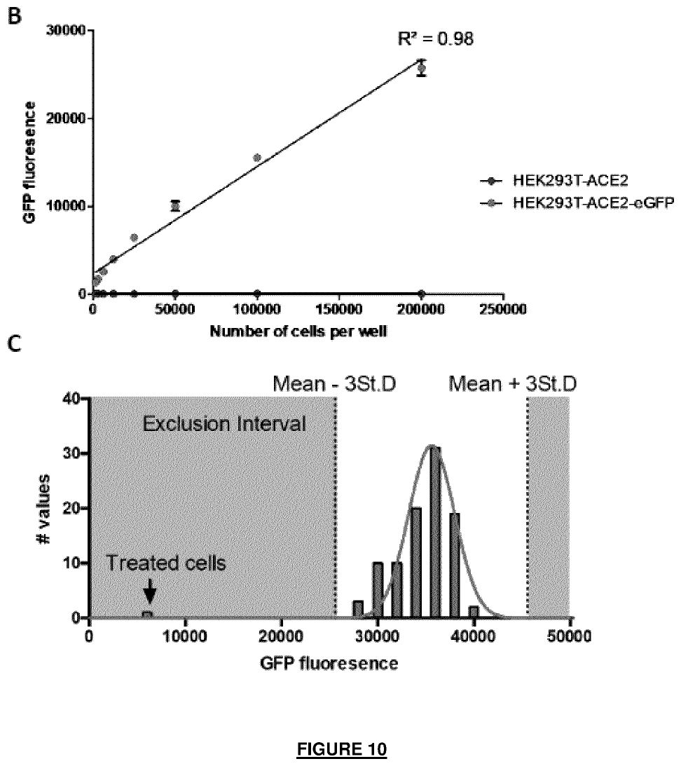

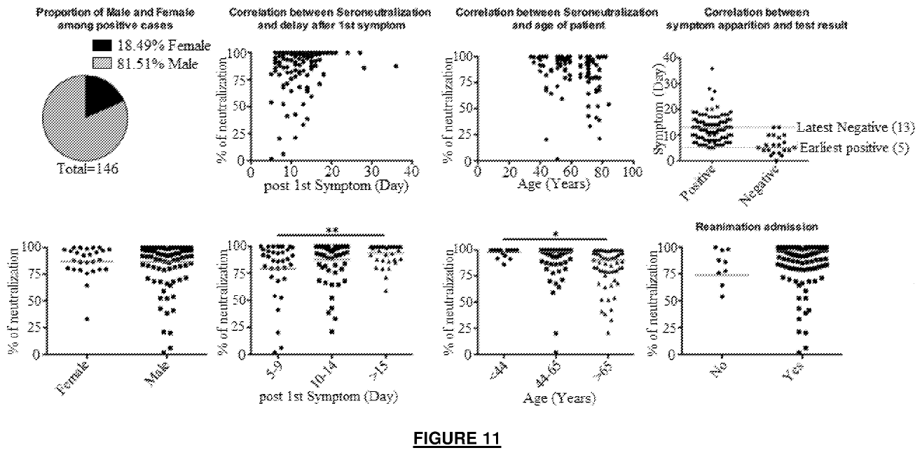

Ranking of the seras in strength, means, and non-neutralizers (FIG. 9).

### Example 11. Analysis and Interpretation of the Bichat Cohort

The activity of seroneutralization appears in a gradual manner, with a plateau after 15 days post-symptoms. (FIG. 11, left part.) Adults show a better rate of neutralization than the elderly (over 65 years old). (FIG. 11, middle part). Patients that needed to go into intensive care show a better neutralization than patients that had less severe symptoms. (FIG. 11, right part.)

### Example 12. Specificity of the Test

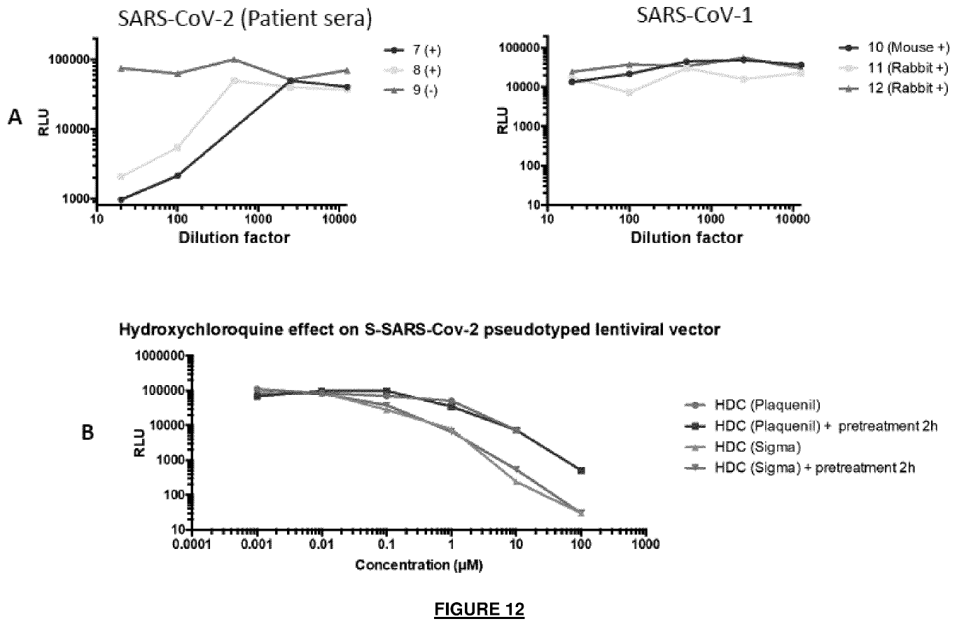

An example Inhibitory dilution curves SARS-CoV-2 infected patient sera and evaluation of cross-reactivity of anti-S Sars-CoV-1 polyclonal antibodies from infected animals is presented in FIG. 12A. The seras of the rabbits and the mouse against the S protein of SARS CoV1 (Hong Kong 2003) did not show any neutralization activity.

### Example 13. Effect of Hydroxychloroquine

Validation of the effect of hydroxychloroquine on the capacity of fusion of viral particles pseudotyped with the S Sars-CoV-2 protein is presented in FIG. 12B.

### Example 14. Detailed Workflow for Robotic Implementation

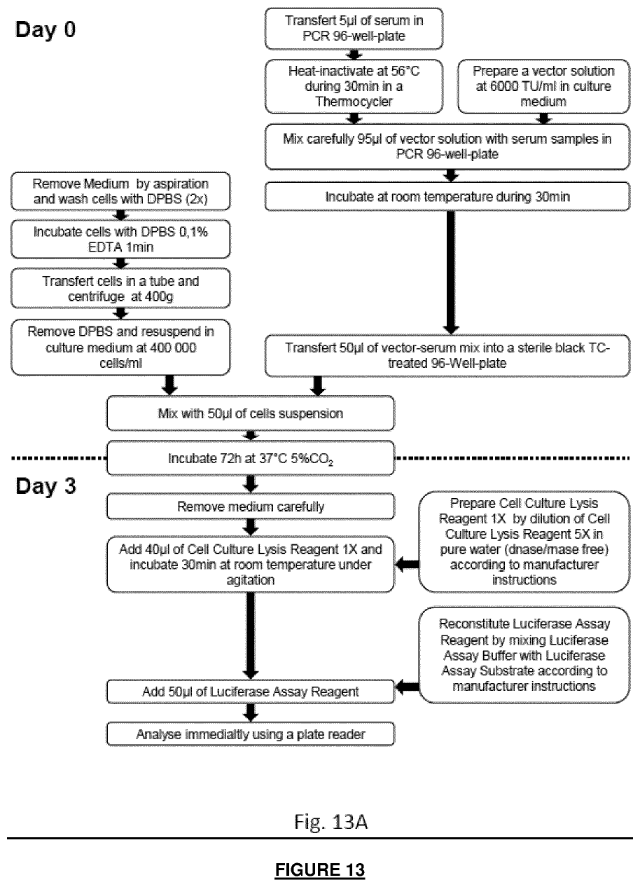

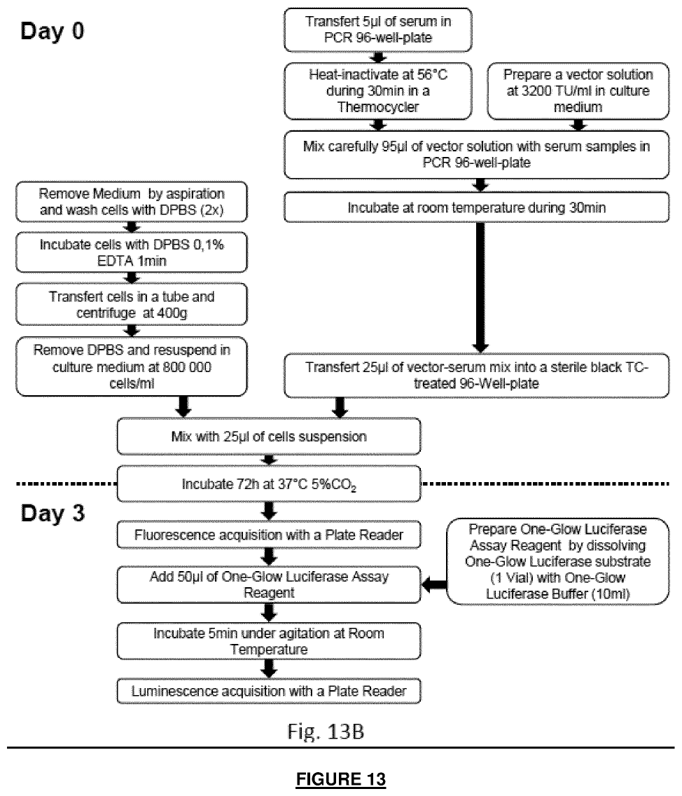

A schematic of the workflow is shown in FIG. 13A (Two-step revelation compatible for HEK293T-hACE2 and HEK293T-hACE2-eGFP) and 13B (One-Step revelation compatible for HEK293T-hACE2-eGFP only)

The workflow shown in FIG. 13B is compatible with Robotic Implementation.

### Example 15: Anti-SARS-CoV-2 Antibody Response in Institut Curie Study Cohort

The remaining examples utilized a different study cohort. Blood samples were collected from 1610 volunteers at the 3 sites of the Institut Curie: Paris (75), Saint Cloud (92) and Orsay (91) from April, 28 until July, 17. None of the individuals showed clinical signs of COVID-19 or had been subjected to a standard RNA detection of SARS-CoV-2, using RT-qPCR, within 14 days prior to blood sampling. All participants were invited to complete a web-based questionnaire which included demographic variables, symptom occurrences and whether these had led to a sick leave, treatment and/or hospitalization. The participant cohort had a strong (77.5%) female bias (Table 1); the mean age was 38 and ranged between 19 and 75 years old. The hospital-working staff represented 74.5% of the volunteers, the rest being researchers and administrative staff.

TABLE 1

| | Whole | | | | | |
|---|---|---|---|---|---|---|
| | Institut Curie | | Hospital | | Research center | |
| | n | % | n | % | n | % |
| Total | 1610 | 100 | 1200 | 74.5 | 410 | 25.5 |
| Female | 1247 | 77.5 | 970 | 80.8 | 277 | 67.6 |
| Male | 363 | 22.5 | 230 | 19.2 | 133 | 32.4 |
| Age (mean) | 38 | | 40 | | 38 | |
| <39 yrs | 822 | 51.1 | 608 | 50.7 | 195.0 | 47.6 |
| >38 yrs | 788 | 48.9 | 592 | 49.3 | 215.0 | 52.4 |
| Lockdown | 716 | 44 | 372 | 31 | 344 | 84 |
| RT-qPCR | 171 | 10.6 | 166 | 13.8 | 5 | 1.2 |
| Positive | 63 | 36.8 | 61 | 36.7 | 2 | 40.0 |
| Serological tests | 1610 | 100 | 1200 | 100 | 410 | 100 |
| IgG/N Positive | 252 | 15.7 | 213 | 17.8 | 39 | 9.5 |
| IgG/S Positive | 162 | 10.1 | 133 | 11.1 | 29 | 7.1 |
| PTN Positive | 154 | 9.6 | 126 | 10.5 | 28 | 6.8 |
| Sero. Positive | 272 | 16.9 | 226 | 18.8 | 46 | 11.2 |
| Female | 207 | 16.6 | 181 | 18.7 | 26 | 9.4 |
| Male | 65 | 17.9 | 45 | 19.6 | 20 | 15.0 |
| <39 yrs | 140 | 17.0 | 113 | 18.6 | 27 | 13.8 |
| >38 yrs | 132 | 16.8 | 113 | 19.1 | 19 | 8.8 |

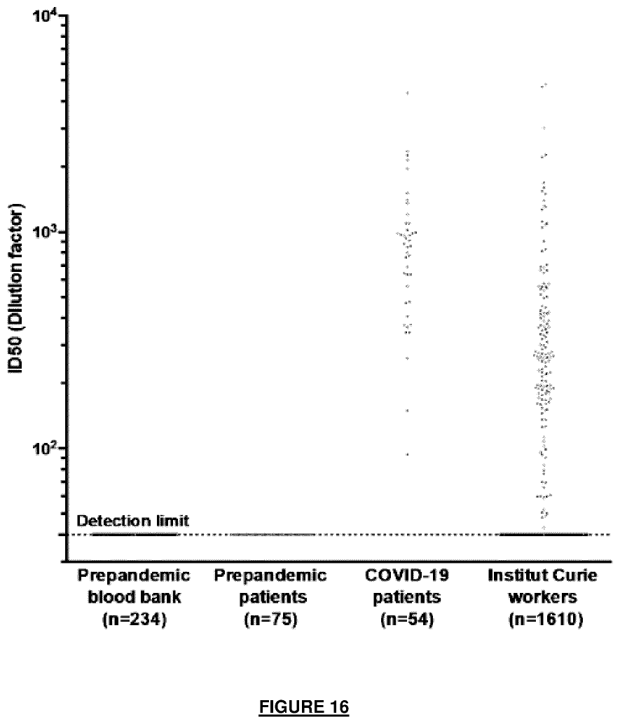

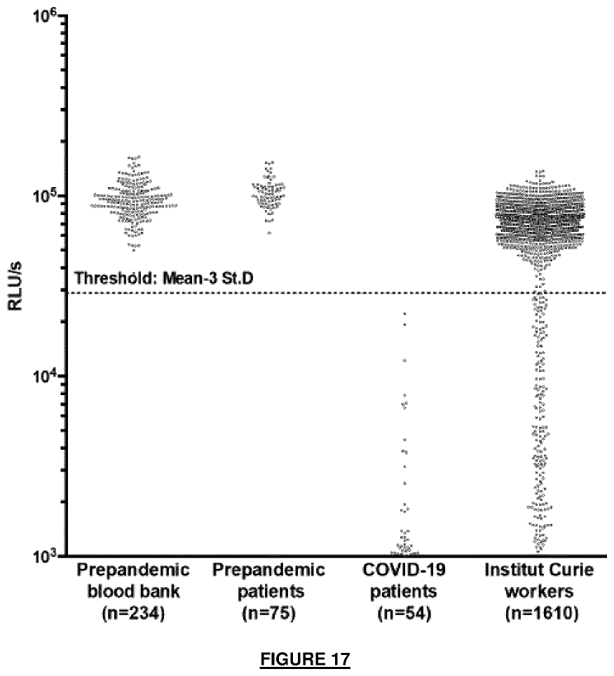

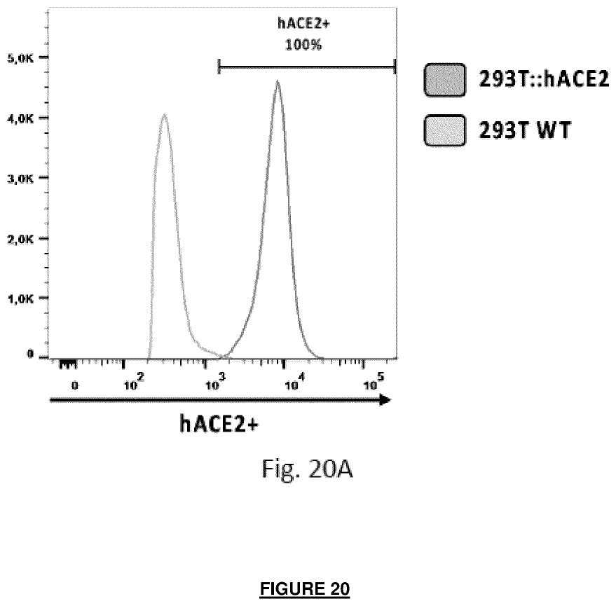

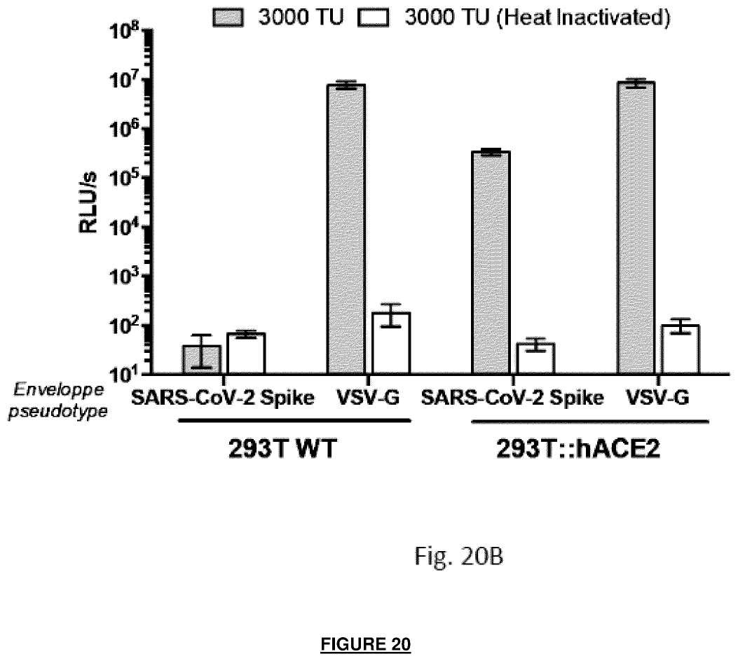

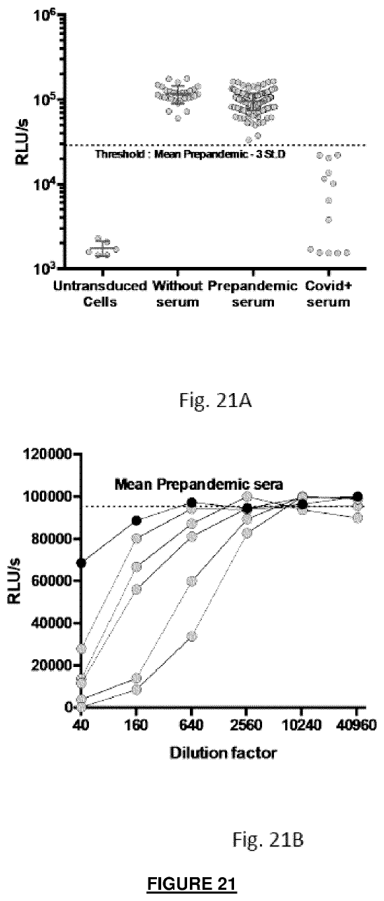

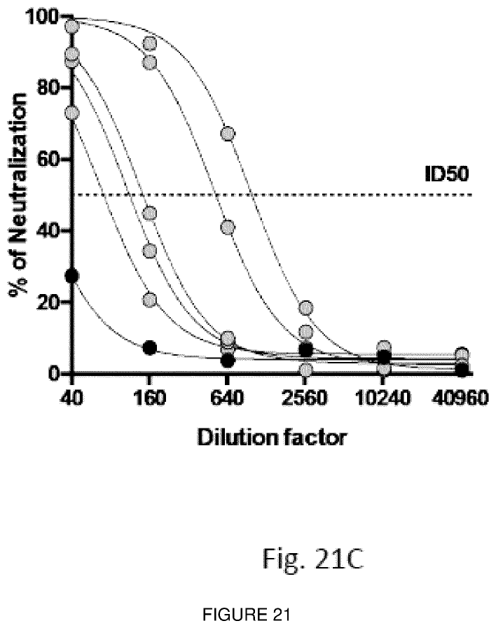

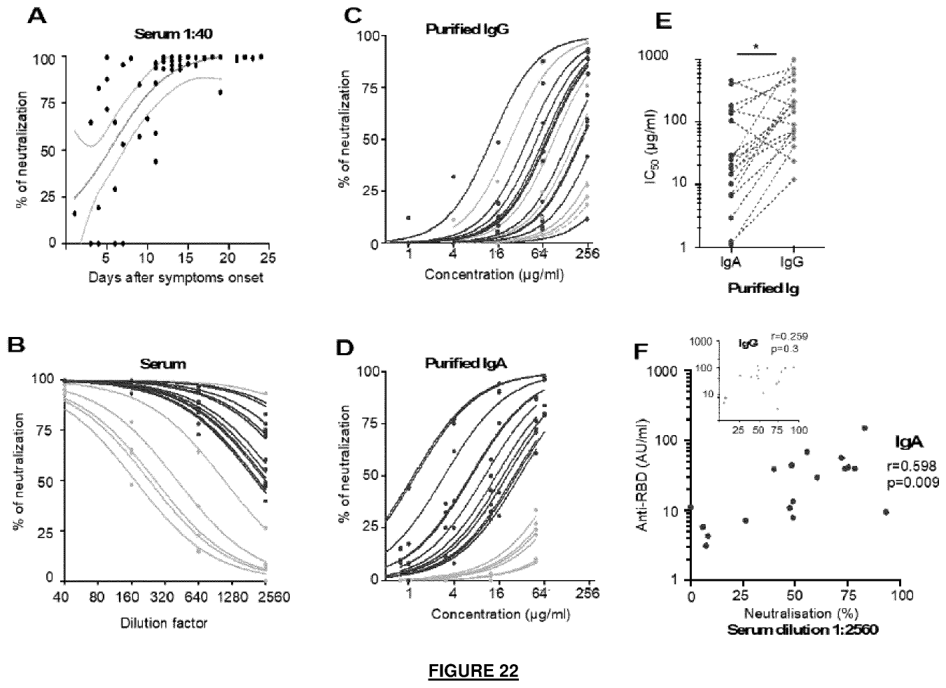

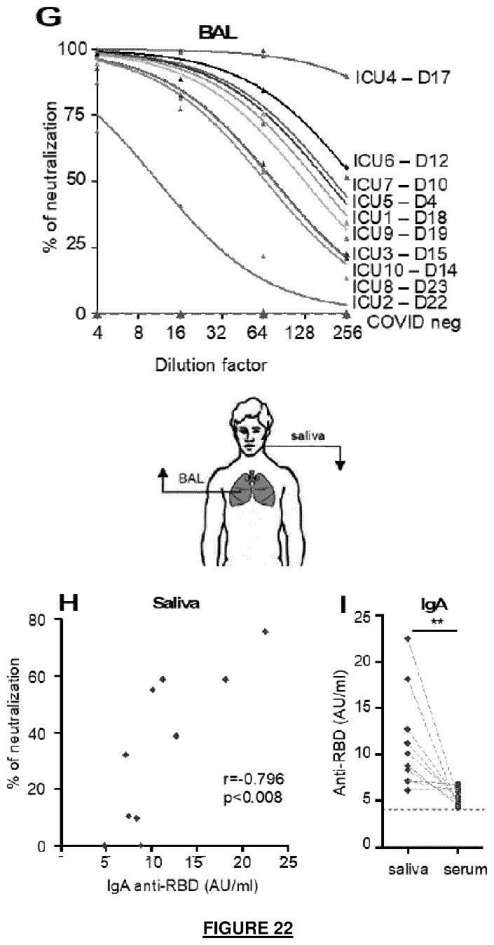

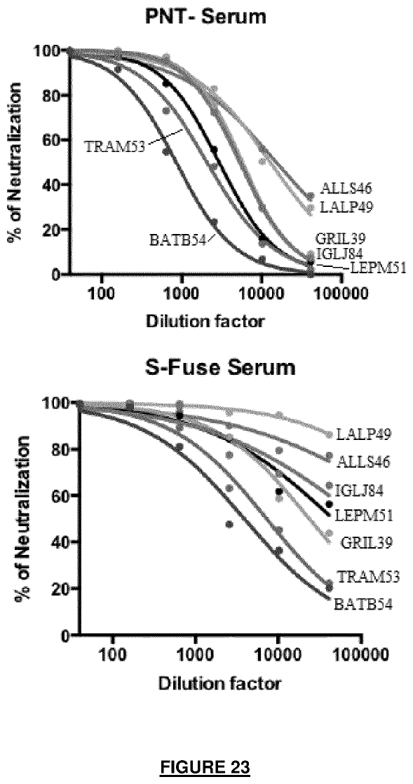

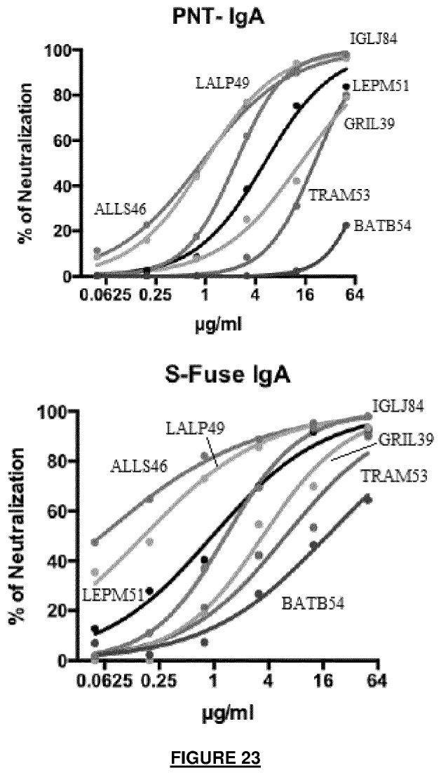

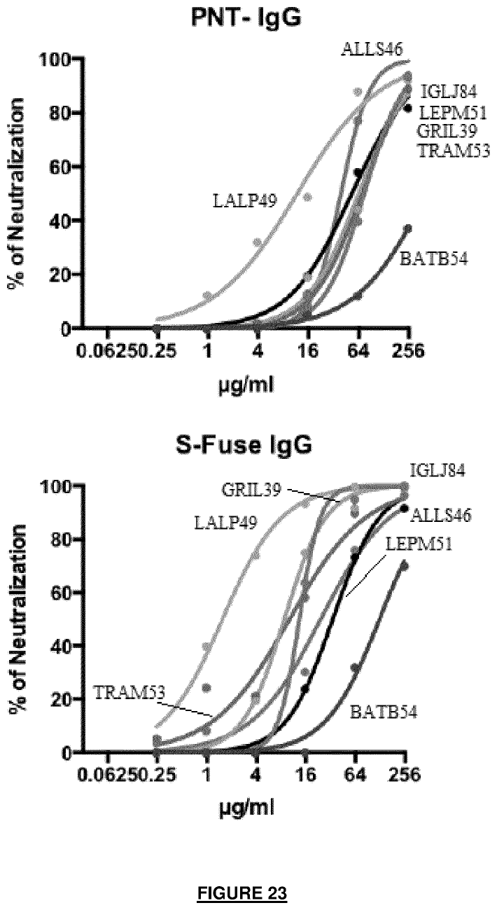

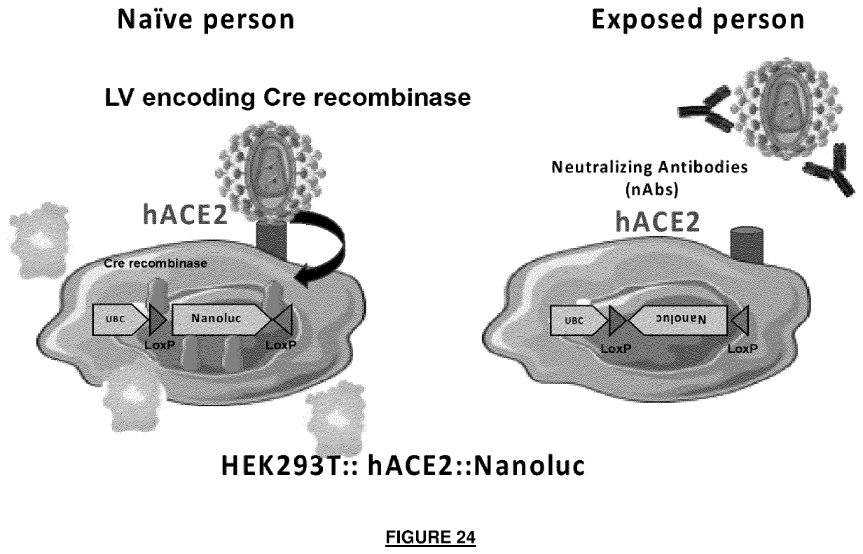

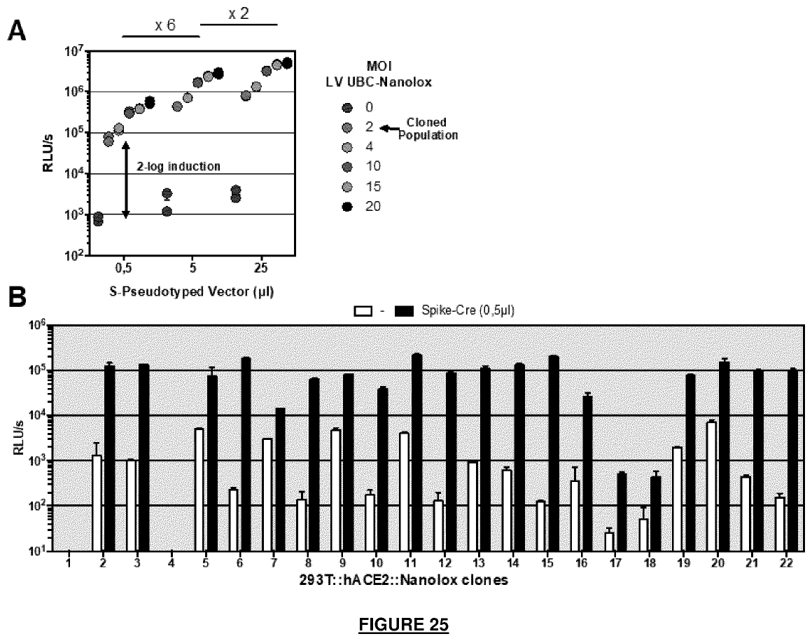

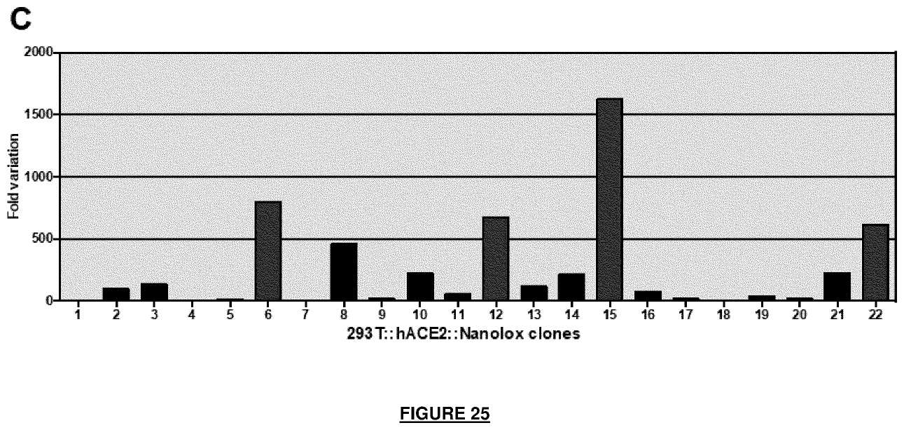

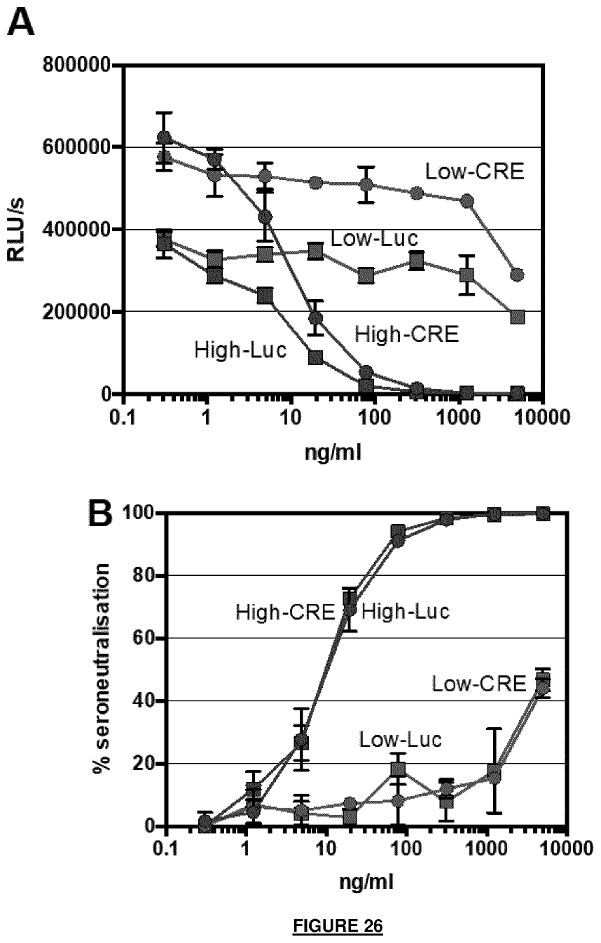

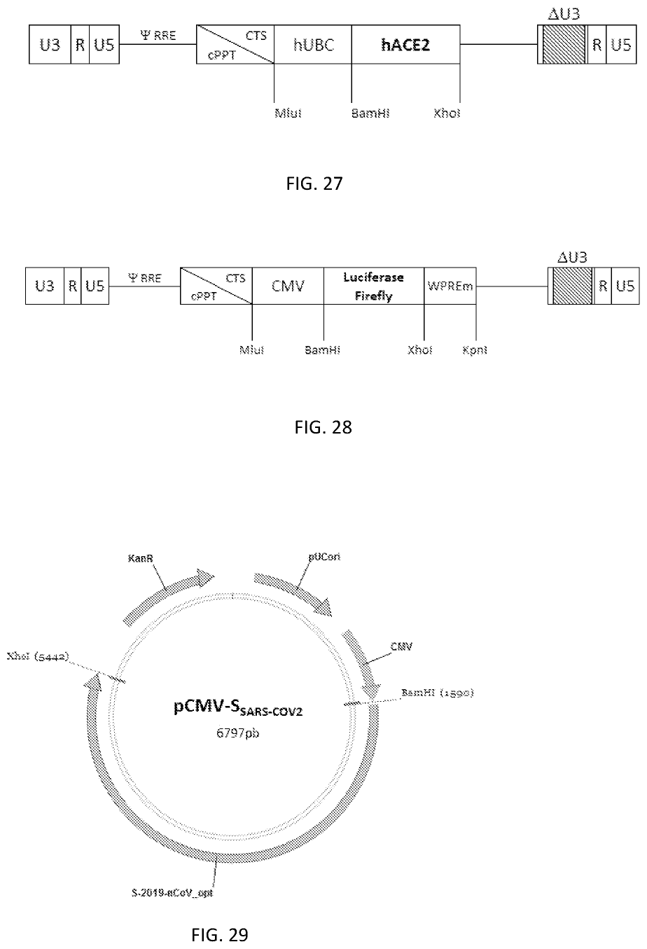

Three serological assays were carried out at the Institut Pasteur in multi-well plates on these 1610 sera samples. Two LuLISA (Luciferase-Linked Immuno-Sorbent Assay)⁸, (not shown), assessed the specific IgG for SARS-CoV-2 Nucleoprotein (N) and Spike (S) proteins. A pseudo-neutralization test (PNT) was also performed⁹, to assess the ability of serum components to neutralize the fusion of a SARS-CoV-2 Spike pseudo-typed lentiviral vector encoding a luciferase gene using a permissive human cell line (HEK 293T) constitutively expressing human ACE2 receptors (FIGS. 20 and 21). The specificity threshold of the three methods were established by using serum samples from 54 COVID-19 patients (March 2020, Institut Cochin), 234 prepandemic negative healthy donors from a blood bank (2014-2018, EFS/ICAReB) and 75 negative serums from prepandemic breast cancer patients (2012, Institut Curie) (FIG. 16). The positivity thresholds were set to 98% specificity for LuLISA assay allowing the detection of anti-N IgG (10,400 RLU/s) and anti-S IgG (8,400 RLU/s) and to a confidence level of 99% for PNT assay (28,783 RLU/s) from prepandemic negative sera.

The robustness of the specificity thresholds and dynamic ranges were assessed using dilution series of COVID-19 positive sera (FIGS. 20 and 21, and not shown). The specificity for SARS-CoV-2 anti-N IgG was assessed against purified Nucleoproteins of SARS-CoV-1 as well as seasonal coronaviruses (HCoV) HKU, OC43, NL63, 229E (not shown).

For the Institut Curie workers, using a 98% specificity threshold, the seroprevalence of IgG directed against N and S proteins was of 15.7% (252/1610, 95% CI: 13.9-17.5) and 10.1% (162/1610, 95% CI: 8.6-11.6), respectively (not shown). Amongst all the serums tested, 9.6% % (154/1610, 95% CI: 7.7-10.5) displayed a pseudo-neutralization activity against the virus (FIG. 16). Considering each of these assays independently as a marker of specific immune response leads to a 16.9% (272/1610, 95% CI: 15.0-18.8) positivity of immunization.

The correlative plots (not shown) indicates that the responses against the N and S are linked when both are above their respective threshold (R²=0.57). Correlation between PNT and LuLISA is mainly detectable when high levels of both IgG against N and S are detected (not shown). Moreover, above the 98% specificity threshold, a higher correlation is observed between PNT and LuLISA IgG/S(R²=0.60) (not shown) than between PNT and LuLISA IgG/N(R²=0.48) (not shown). Remarkably, out of the 272 seropositive samples, only 50% are positive for the 3 assays and 37% are only positive for LuLISA IgG/N (not shown).

### Example 16: Prevalence and Linetic of Symptoms and Serological Responses

Based on the web-based survey, 57% (921/1610) participants mentioned at least one symptom. Symptomatic workers were more seropositive (23%, 211/921, CI 95%: 20.3-25.9) than asymptomatic workers (9.1%, 63/689, CI 95%: 7.1-11.5) (Table 2). Hence, SARS-CoV-2 infection may have been asymptomatic in at least 23.2% (63/272, 95% CI: 18.3-28.6) of the cases (not shown). The amount of anti-N antibodies was higher in the symptomatic versus asymptomatic patients while the levels of anti-S or the pseudo-neutralization capacity did not differ (not shown). This discrepancy suggests that anti-N IgG may even be generated in the course of a mild infection.

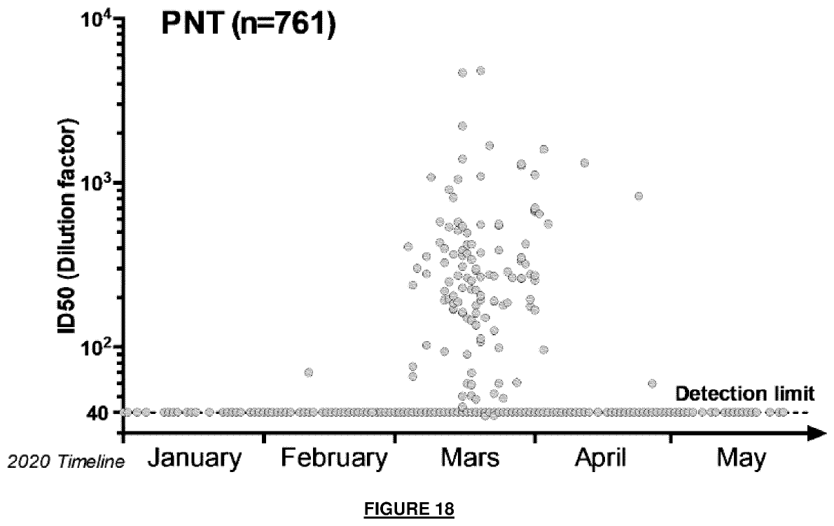

A date for the symptom onset was mentioned in 770 out of 921 cases. Symptoms were mostly (63%) reported in March, 2020 consistent with the reported epidemic development as well as the number of Parisian hospital admissions published daily by Santé Publique France¹⁰ (not shown). The intensity of immune responses according to the date of symptom occurrence is reported in FIG. 18 for PNT and not shown for others. The decrease seen in April (15%) probably reflects the efficacy of the population lock down on the disease spread. The March peak of symptom occurrence represented 78% of the seropositive individuals compared to 57% in people devoid of COVID-19 specific IgG. Although some workers displayed an immune response corresponding to symptoms dated as early as the first week of February, 2020, a sharp peak of seropositive individuals corresponded to symptoms declared in March (73% of the cases). These results indicate that the virus was circulating in early February in the Paris conurbation and achieved a high prevalence in March.

The frequency of declared symptoms was significantly much higher in seropositive workers than in those devoid of COVID-19 specific IgG (Table 2). If tiredness (64%, 174/272) and unusual headache (54%, 146/272) were the most frequent symptoms in the seropositive population they were also noted in individuals lacking antibodies (37%, 495/1338 and 34%, 458/1338 respectively) suggesting a low correlation with a COVID-19 infection (Chi-square scores 1E⁻¹⁸ and 3E⁻¹²) (Table 2). To the opposite, anosmia/ageusia and myalgia symptoms were highly prevalent (41%, 111/272 and 42%, 115/272 respectively) in the seropositive group but were also rare in the seronegative group 3% (39/1338) and 16% (215/1338) respectively (not shown), resulting in a high correlation with COVID-19 (chi-square scores 5E⁻⁷³ and 3E⁻²¹) (Table 2). Only anosmia/ageusia symptoms were temporally correlated with the epidemic peak in March whereas other symptoms such as myalgia and rhinitis (not shown) were declared by seronegative workers mainly before but also after this peak suggesting the effects of other circulating diseases.

A correlation between serological tests, RT-qPCR and symptoms was performed (not shown). In the 174 individuals tested by RT-qPCR, 99% reported symptoms, only 79 (45%, CI 95%: 37.9-53.1) were positive in serological or RT-qPCR-based assays. Moreover, no IgG antibodies were detected in 3 subjects out of 63 with a positive SARS-CoV-2 RT-qPCR indicating that a systemic IgG response (against N or S proteins) may not always be present following a SARS-CoV-2 proven infection (not shown). However, low levels of anti-SARS-CoV-2 IgM, were detected using a commercial lateral flow assay, in 1 of these 3 subjects (data not shown). Anti-N IgM peaked at day 9 after disease onset and then switched to IgG by week 2¹¹. Except for one case, all anosmia/ageusia cases without detectable systemic IgG (n=39) were associated with other COVID-19 typical symptoms and occurred in late February, March or April suggesting that they represent true SARS-CoV-2 infections. Indeed, one of them was associated with a positive SARS-CoV-2 RT-qPCR test and, in 8 cases anti-SARS-CoV-2 IgM were detected using a lateral flow assay (data not shown). Thus, in addition to the 272 SARS-CoV-2 immune cases detected by our survey, the cohort may feature an additional 38 infection cases devoid of detectable systemic IgG antibodies. Assuming that the incidence (41%) of the anosmia/ageusia symptom is similar in immune and non-immune individuals the true prevalence of SARS-CoV-2 infection in this population would then be more than 16.9% (272/1610) and as high as 22.7% ((272+(38/0.41)=365); 365/1610).

TABLE 2

| | Total | | Seroposivitive | | Seronegative | | |
|---|---|---|---|---|---|---|---|
| | (1610) | | (272) | | (1338) | | |
| | n | % | n | % | n | % | p (Chi2) |
| Any symptom | 921 | 57 | 209 | 77 | 712 | 53 | 7E−13 |
| Anosmia, ageusia | 150 | 9 | 111 | 41 | 39 | 3 | 5E−73 |
| Myalgia | 330 | 22 | 115 | 42 | 215 | 16 | 3E−21 |
| Tiredness | 669 | 42 | 174 | 64 | 495 | 37 | 1E−18 |
| Fever | 275 | 17 | 95 | 35 | 180 | 13 | 8E−17 |
| Cough | 373 | 23 | 107 | 39 | 266 | 20 | 9E−13 |
| Unusual Headache | 604 | 38 | 146 | 54 | 458 | 34 | 4E−12 |
| Shortness of breath | 217 | 14 | 70 | 26 | 147 | 11 | 1E−10 |
| Rhinitis | 348 | 22 | 92 | 34 | 256 | 19 | 6E−09 |
| Intestinal symptoms | 232 | 14 | 57 | 21 | 175 | 13 | 8E−04 |
| Conjunctivitis | 85 | 5 | 23 | 8 | 62 | 5 | 6E−03 |

### Example 17: Decrease of Antibody Titer and Neutralization Activity with Time

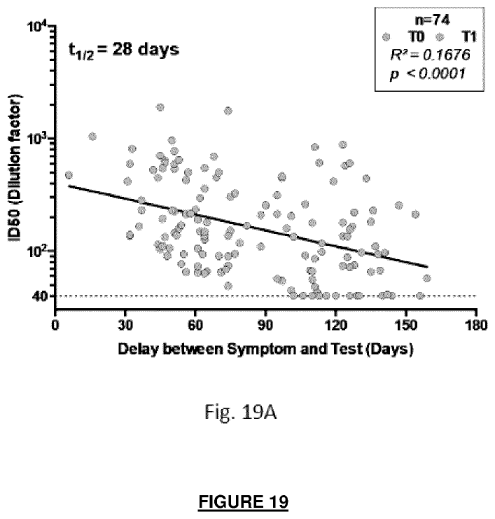

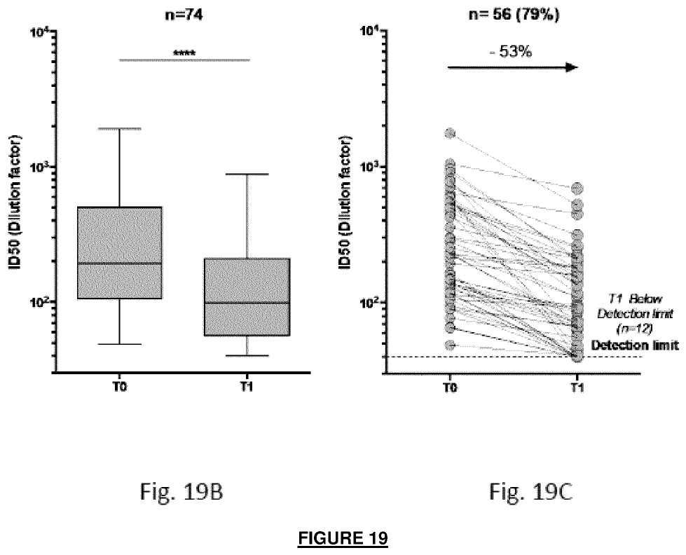

To follow over time the antibody titers and neutralizing activity, a second blood sample (t₁) was obtained 4-8 weeks after the first one (t₀) for more than 1000 individuals. The results, for the 120 samples of individuals previously found positive, are reported in FIG. 19A (for the PNT assay; others not shown) according to the time-interval between symptom onset and sampling. A clear decrease in the antibody titers and virus pseudo-neutralization capacity was observed. The half-lives of the antibody titers were 35, 87 and 28 days for anti-N, anti-S IgG and pseudo-neutralization, respectively. A paired analysis showed a systematic decreased response (p<0.0005) (FIG. 19B for the PNT assay; others not shown). The titers of antibodies decreased by 31% and 17% for anti-N and anti-S IgG, respectively for a majority of workers (>75%) and this correlated with a major decrease of the ID50 pseudo-neutralization capacity (53%) (FIG. 19C for the PNT assay; others not shown). Interestingly, some workers sera became negative in the assays: 15% (16/107) for LuLISA IgG/N (not shown), 14% (10/71) for PNT (FIG. 19C) and 5% (4/84) for LuLISA IgG/S (not shown). Thus, after a few months, a serological-based survey of SARS-CoV-2 may run a risk of underestimating the number of formerly infected individuals.

### Example 18. Discussion

Beyond the simple detection of individuals that have been in contact with the SARS-COV-2 virus, the knowledge of immune protection (or on the contrary facilitation in case of re-infection in individuals with low antibody responses) detected with sensitive tests is key to avoid misuse of serological tests. Neutralizing antibodies have a major role in preventing reinfections for many viral diseases. A major point is the relationship between in vivo protection and the levels of antibody binding to the virus or neutralizing it. We compared multiple serological assays to MNT and pseudovirus neutralization assay. We observed a strong correlation between the extent of anti-full S and even anti-N response and the neutralization capacity of the sera.

The seroneutralization assays described herein will also be critically useful to further characterize a patient's protection and also to characterize alternative seroconversion and other antibody assays.

Non-neutralizing antibodies, or neutralizing antibodies at sub-optimal doses can also lead to Antibody-Dependent Enhancement of infection (ADE). ADE exacerbates diseases caused by feline coronavirus, MERS-CoV and SARS-CoV-1²⁵⁻²⁸. ADE might thus also play a deleterious role in COVID-19. The various techniques described here are instrumental to determine the serological status of individuals or populations and establish potential correlates of disease facilitation or protection.

### Example 19: Additional Method According to the Invention

Vector Production

The production of a lentiviral vector pseudotyped by the SARS-CoV-2 spike protein expressing CRE protein follows a standard lentiviral vector production protocol as described above, mutatis mutandis.

The reagents used are (i) packaging plasmid unmodified p8.74; (ii) transfer plasmid: pTRIP with a transcriptional unit composed of CRE recombinase ORF (from Phage P1 of E. coli) under the control of a human ieCMV promoter; and (iii) envelop plasmid: a modified pCMV-VSV-G with an SV40 replication site and the codon optimized Spike Sars-CoV-2 cdna in place of VSV-G. The productions are standardized at 3 μg/ml of p24 after quantification by ELISA.

Briefly, 1×10⁷ HEK293T cells/Petri dish (100 mm) are cultured in DMEM 10% FCS+pen/strep and were transfected with 1 ml of a mixture of: (i) 10 μg/ml of the p8.74 packaging plasmid, encoding for codon optimized gag-pol-tat-rev, (ii) 5 μg/ml of envelop plasmid, and (iii) 10 μg/ml of “transfer” pTRIP plasmid in Hepes 1× containing 125 mM of Ca(ClO3)2. At 24 h, medium is renewed. At 48 h, Supernatants were harvested and clarified by 6-minute centrifugation at 2500 rpm.

Generation of HEK293T-hACE2-Nanolox Cell Line

A lentiviral vector (pseudotyped VSV-G) comprising a transcriptional unit with the constitutive human ubiquitin-C promoter and the optimized codon ORF was used to transduce HEK293T (MOI 10) and cloned by limiting dilution. Then a second transduction (MOI 1) was performed with a lentiviral vector ((pseudotyped VSV-G) comprising a transcriptional unit with the constitutive human ubiquitin-C promoter and a inverted Nanoluc ORF flanked with two LoxP sites in opposite direction. A clone was selected by limiting dilution.

The line was selected on the level of expression of the nanoluc reporter after a transduction testing with the Spike pseudotyped vector expressing CRE protein.

Pseudo-Neutralisation Assay

The reagents implemented are:
PCR plates (Biorad #HSP9601);
162 cm² Flask (CORNING #3151);
Tubes 50 ml (Falcon #352098);
DPBS1× (GIBCO #14199-094);
DPBS+EDTA 0.1%: Dissolve 1 g of EDTA (Promega #H5032) in 1 liter of DPBS1×;
0.05% Trypsin-EDTA (GIBCO #25300-054);
96-well Flat Clear Bottom White Polystyrene TC-treated Microplates, (Costar #3610) Note: If the signal is too strong for the sensor of the reader even with the shortest acquisition time, it can be considered to use black plates, although less optimal;
Revelation solution (Hikarazin or Furimazine)
DMEM High Glucose, HEPES, No Phenol, L-Glutamine 500 ml (GIBCO #21063-029)
Sodium Pyruvate (GIBCO #11360-070)
Foetal Calf Serum (FCS) 500 ml (CORNING #35-079-CV)
Antibiotic Pen/Strep 100× (GIBCO #15140-122)

Medium Preparation:
DMEM High Glucose, HEPES, No Phenol, L-Glutamine 500 ml
+Sodium Pyruvate 100 mM 5 ml
+Fetal calf serum 50 ml

Culture Guidelines for HEK293T-hACE2-Nanolox Cells:

Grow cells between 10M and 70M cells in 162 cm² flasks. Maintain cells every 3-4 days by passage of cells through trypsinization. Keep cells for a maximum of 10 passages.

Preparation of the 293T-hACE2-Nanolox Cell Suspension:
Preheat the culture medium, the DPBS and the 0.1% DPBS-EDTA at 37° C. in a water bath for 15 min.
Remove the culture medium
Wash cells 1 time with 10 mL of DPBS
Add 10 ml of 0.1% DPBS-EDTA

When preparing for sero-neutralization, trypsin must not be used as it cleaves the hACE2 receptor.
Incubate at 37° C. 1 min
Peel off the cells by tapping the flask, then delicately individualize them with a 10 mL pipette by successive aspiration/delivery
Recover the cells in a 50 ml Falcon tube
Centrifuge 400 g for 5 min
Remove 0.1% DPBS-EDTA.
Resuspend at 400,000 cells/mL in culture medium.
Store at 37° C. until use.

PBS-EDTA 0.1% treated cells are not to be used for culture maintenance.

Preparation of the Vector Solution:

The culture medium is warmed to room temperature. The SARS-CoV-2 pseudotyped vector solution is diluted to 1/50 in culture medium at RT. It is mixed gently (no vortex). Then the vector solution is immediately used after preparation.

If the stock vector solution is in large volume: it must be aliquoted after the 1st thawing according to the number of tests conventionally carried out (count 0.5 μl per test). Vector aliquots must never be refreezed. The diluted vector solution must never be stored.

Preparation of Serum Dilutions:

Serum is heat-inactivated at exactly 56° C. for 30 minutes with a thermocycler in a DNA/RNAfree PCR plate. This step is crucial to ensure the viability of the cells during the test. 10 μl per EC50 (duplicate) must be counted.

If several variants are tested, the volumes must be multiplied accordingly.

A 96-well round-bottom plate must be prepared with 90 μL of medium in the first row then 75 μL in the following 5 rows.

Transfer 10 the sera to the first line and carry out serial dilutions with 25 μL: a first dilution at 1/10 then at 1/4 to 1/10240th (6 dilutions).

A white culture plate must be prepared with 25 μL of the vector solution per well. 25 μL of diluted serum must be transferred and mixed. Incubation lasts 30 min at RT. 50 μl of the cell suspension are then added (20 000 Cells/wells). Incubation is then performed 72 h at 37° C. 5° CO2.

Read-Out

The Revelation solution is prepared according to the manufacturer's instructions. 50 μL of solution are added per well. We then wait 5 min before reading.

As illustrated in FIG. 25, the functionality of the Cre-Nanolox system in a pseudo-seroneutralisation assay has been accordingly validated through observation of the number of photons produced per second (RLU/s) when the quantity of S-pseudotyped vectors comprising the sequence encoding the CRE recombinase increased.

Moreover, as illustrated in FIG. 26, this system has also been validated and compared to the previously described system implementing viral particles pseudotyped with the SARS-CoV-2 protein S (Wuhan strain) comprising lentiviral vector expressing Firefly luciferase, using either a monoclonal antibody having a high affinity for Spike (Wuhan strain) (high) or a monoclonal antibody having a low affinity for Spike (low).

### REFERENCES CITED

1. Wu F, Zhao S, Yu B, et al. A new coronavirus associated with human respiratory disease in China. Nature 2020; 579(7798): 265-9.
2. Zhou P, Yang X L, Wang X G, et al. A pneumonia outbreak associated with a new coronavirus of probable bat origin. Nature 2020; 579(7798): 270-3.
3. Corman V M, Landt O, Kaiser M, et al. Detection of 2019 novel coronavirus (2019-nCoV) by real-time RT-PCR. Eurosurveillance 2020; 25(3): 2000045.
4. Chu D K W, Pan Y, Cheng S M S, et al. Molecular Diagnosis of a Novel Coronavirus (2019-nCoV) Causing an Outbreak of Pneumonia. Clinical Chemistry 2020.
5. Pfefferle S, Reucher S, Nörz D, LOtgehetmann M. Evaluation of a quantitative RT-PCR assay for the detection of the emerging coronavirus SARS-CoV-2 using a high throughput system. Eurosurveillance 2020; 25(9): 2000152.
6. Wölfel R, Corman V M, Guggemos W, et al. Virological assessment of hospitalized patients with COVID-2019. Nature 2020.
7. To K K, Tsang O T, Leung W S, et al. Temporal profiles of viral load in posterior oropharyngeal saliva samples and serum antibody responses during infection by SARS-CoV-2: an observational cohort study. Lancet Infect Dis 2020.
8. Hoffmann M, Kleine-Weber H, Krüger N, Müller M, Drosten C, Pöhlmann S. The novel coronavirus 2019 (2019-nCoV) uses the SARS-coronavirus receptor ACE2 and the cellular protease TMPRSS2 for entry into target cells. Biorxiv 2020: 2020.01.31.929042.
9. Li F, Li W, Farzan M, Harrison S C. Structure of SARS Coronavirus Spike Receptor-Binding Domain Complexed with Receptor. Science 2005; 309(5742): 1864-8.
10. Song W, Gui M, Wang X, Xiang Y. Cryo-EM structure of the SARS coronavirus spike glycoprotein in complex with its host cell receptor ACE2. PLoS Pathog 2018; 14(8): e1007236.
11. Yan R, Zhang Y, Li Y, Xia L, Guo Y, Zhou Q. Structural basis for the recognition of SARS-CoV-2 by full-length human ACE2. Science 2020; 367(6485): 1444-8.
12. Walls A C, Xiong X, Park Y J, et al. Unexpected Receptor Functional Mimicry Elucidates Activation of Coronavirus Fusion. Cell 2019; 176(5): 1026-39 e15.
13. Amanat F, Nguyen T, Chromikova V, et al. A serological assay to detect SARS-CoV-2 seroconversion in humans. medRxiv 2020: 2020.03.17.20037713.
14. OKBA NMA, Muller M A, Li W, et al. SARS-CoV-2 specific antibody responses in COVID-19 patients. medRxiv 2020: 2020.03.18.20038059.
15. Zhao J, Yuan Q, Wang H, et al. Antibody responses to SARS-CoV-2 in patients of novel coronavirus disease 2019. Clin Infect Dis 2020.
16. Guo L, Ren L, Yang S, et al. Profiling Early Humoral Response to Diagnose Novel Coronavirus Disease (COVID-19). Clinical Infectious Diseases 2020.
17. Jiang H-w, Li Y, Zhang H-n, et al. Global profiling of SARS-CoV-2 specific IgG/IgM responses of convalescents using a proteome microarray. medRxiv 2020: 2020.03.20.20039495.
18. Muller M A, Meyer B, Corman V M, et al. Presence of Middle East respiratory syndrome coronavirus antibodies in Saudi Arabia: a nationwide, cross-sectional, serological study. Lancet Infect Dis 2015; 15(6): 629.
19. Ou X, Liu Y, Lei X, et al. Characterization of spike glycoprotein of SARS-CoV-2 on virus entry and its immune cross-reactivity with SARS-CoV. Nature Communications 2020; 11(1): 1620.
20. Wu F, Wang A, Liu M, et al. Neutralizing antibody responses to SARS-CoV-2 in a COVID-19 recovered patient cohort and their implications. medRxiv 2020: 2020.03.30.20047365.
21. Poh C M, Carissimo G, Wang B, et al. Potent neutralizing antibodies in the sera of convalescent COVID-19 patients are directed against conserved linear epitopes on the SARS-CoV-2 spike protein. Biorxiv 2020: 2020.03.30.015461.
22. Ju B, Zhang Q, Ge X, et al. Potent human neutralizing antibodies elicited by SARS-CoV-2 infection. Biorxiv 2020: 2020.03.21.990770.
23. Li R, Pei S, Chen B, et al. Substantial undocumented infection facilitates the rapid dissemination of novel coronavirus (SARS-CoV2). Science 2020.
24. Lescure F-X, Bouadma L, Nguyen D, et al. Clinical and virological data of the first cases of COVID-19 in Europe: a case series. The Lancet Infectious Diseases 2020.
25. Hohdatsu T, Yamada M, Tominaga R, Makino K, Kida K, Koyama H. Antibody-Dependent Enhancement of Feline Infectious Peritonitis Virus Infection in Feline Alveolar Macrophages and Human Monocyte Cell Line U937 by Serum of Cats Experimentally or Naturally Infected with Feline Coronavirus. Journal of Veterinary Medical Science 1998; 60(1):49-55.
26. Kam Y W, Kien F, Roberts A, et al. Antibodies against trimeric S glycoprotein protect hamsters against SARS-CoV challenge despite their capacity to mediate FcγRII-dependent entry into B cells in vitro. Vaccine 2007; 25(4): 729-40.
27. Wan Y, Shang J, Sun S, et al. Molecular Mechanism for Antibody-Dependent Enhancement of Coronavirus Entry. Journal of Virology 2020; 94(5): e02015-19.
28. Wang S-F, Tseng S-P, Yen C-H, et al. Antibody-dependent SARS coronavirus infection is mediated by antibodies against spike proteins. Biochemical and Biophysical Research Communications 2014; 451(2): 208-14.
29. Esterre P, Ait-Saadi A, Arowas L, et al. The ICAReB Platform: A Human Biobank for the Institut Pasteur and Beyond. Open Journal of Bioresources 2020; 7(1): 1.
30. Lorin V, Mouquet H. Efficient generation of human IgA monoclonal antibodies. J Immunol Methods 2015; 422:102-10.
31. Elbe S, Buckland-Merrett G. Data, disease and diplomacy: GISAID's innovative contribution to global health. Glob Chall 2017; 1(1): 33-46.
32. Temmam S, Chretien D, Bigot T, et al. Monitoring Silent Spillovers Before Emergence: A Pilot Study at the Tick/Human Interface in Thailand. Front Microbiol 2019; 10: 2315.
33. Iglesias M C, Mollier K, Beignon A S, et al. Lentiviral vectors encoding HIV-1 polyepitopes induce broad CTL responses in vivo. Mol Ther 2007; 15(6):1203-10.

1. A pseudotyped lentiviral vector particle bearing a SARS-CoV-2 S protein.

2. The pseudotyped lentiviral vector particle of claim 1, wherein the SARS-CoV-2 S protein has an amino acid sequence at least 95% identical to SEQ ID NO: 1.

3. The pseudotyped lentiviral vector particle of claim 1, wherein the SARS-CoV-2 S protein is expressed from a coding sequence having a nucleotide sequence at least 80% identical to SEQ ID NO: 2.

4. The pseudotyped lentiviral vector particle according to claim 1, further comprising a heterologous polynucleotide that encodes a label.

5. The pseudotyped lentiviral vector particle according to claim 4, wherein the label is a fluorescent protein or an enzyme.

6. (canceled)

7. A composition, kit or system, comprising a pseudotyped lentiviral vector particle according to claim 1, and a mammalian cell expressing an Angiotensin-converting Enzyme 2 (ACE2) protein.

8. The composition, kit or system according to claim 7, wherein the mammalian cell further expresses the serine protease TMPRSS2.

9. The composition, kit or system according to claim 7, wherein the mammalian cell is a human cell.

10. The composition, kit or system according claim 9, wherein the human cell is a 293T cell or a HeLa cell.

11. The composition, kit or system according to claim 7, wherein the ACE2 protein has an amino acid sequence at least 95% identical to SEQ ID NO: 3.

12. The composition, kit or system according to claim 7, wherein the ACE2 protein is expressed from a coding sequence having a nucleotide sequence at least 80% identical to SEQ ID NO: 4.

13. The composition, kit or system according to claim 7, further comprising a human serum.

14. The composition, kit or system according to claim 7, further comprising a SARS-CoV-2 S protein binding agent.

15. The composition, kit or system according to claim 7, further comprising an ACE2 binding agent.

16. The composition, kit or system according to claim 7, further comprising reagents for visualizing the label.

17. (canceled)

18. (canceled)

19. A method of assaying for the presence of neutralizing antibodies against a SARS-CoV-2 S protein in a sample comprising antibodies comprising:
a) providing pseudotyped lentiviral vector particles bearing a SARS-CoV-2 S protein and comprising a heterologous polynucleotide that encodes a label;
b) providing mammalian cells expressing an ACE2 protein;
c) contacting the pseudotyped lentiviral vector particles bearing a SARS-CoV-2 S protein with the sample comprising antibodies;
d) contacting the mammalian cells expressing an ACE2 protein with the pseudotyped lentiviral vector particles bearing a SARS-CoV-2 S protein; and
e) assaying for the presence of the label in the mammalian cells.

20. A method of assaying for the presence of neutralizing antibodies against a SARS-CoV-2 S protein in a sample comprising antibodies comprising:
a) providing pseudotyped lentiviral vector particles bearing a SARS-CoV-2 S protein and comprising a heterologous polynucleotide that encodes a recombinase;
b) providing mammalian cells expressing an ACE2 protein and comprising a nanolox nucleotide sequence comprising or consisting of a sequence that is at least 90%, 91%, 92%, 93%, 94%, 95%, 96%, 97%, 98%, or 99% identical to SEQ ID NO: 11;
c) contacting the pseudotyped lentiviral vector particles bearing a SARS-CoV-2 S protein with the sample comprising antibodies;
d) contacting the mammalian cells expressing an ACE2 protein with the pseudotyped lentiviral vector particles bearing a SARS-CoV-2 S protein and comprising the heterologous polynucleotide that encodes the recombinase; and
e) assaying for the presence of the expression of the Nanoluc protein encoded in the nanolox nucleotide sequence in the mammalian cells.

21. The method according to claim 19, wherein c) and d) occur sequentially or simultaneously.

22. (canceled)

23. The method according to claim 19, wherein c) comprises incubating the pseudotyped lentiviral vector particles bearing a SARS-CoV-2 S protein with the sample comprising antibodies for i) at least 15 minutes or ii) from 30 to 60 minutes prior to performing d).

24. (canceled)

25. The method according to claim 19, wherein d) comprises incubating the mammalian cells expressing an ACE2 protein with the pseudotyped lentiviral vector particles bearing a SARS-CoV-2 S protein for from 48 to 72 hours.

26. The method according to claim 19, wherein the mammalian cells in d) are p adhered to a solid support or ii) in a suspension culture.

27. (canceled)

28. The method according to claim 19, wherein the SARS-CoV-2 S protein has an amino acid sequence at least 95% identical to SEQ ID NO: 1.

29. The method according to claim 19, wherein the SARS-CoV-2 S protein is expressed from a coding sequence having a nucleotide sequence at least 80% identical to SEQ ID NO: 2.

30. The method according to any one of claims 19 and 21 to 28, wherein the label is a fluorescent protein or an enzyme.

31. (canceled)

32. The method according to claim 19, wherein the mammalian cells further express the serine protease TMPRSS2.

33. The method according to claim 19, wherein the mammalian cells are human cells.

34. The method according to claim 33, wherein the human cells are 293T cells or a HeLa cells.

35. The method according to claim 19, wherein the ACE2 protein has an amino acid sequence at least 95% identical to SEQ ID NO: 3.

36. The method according to claim 19, wherein the ACE2 protein is expressed from a coding sequence having a nucleotide sequence at least 80% identical to SEQ ID NO: 4.

37. The method according to claim 19, wherein the sample is human serum.

38. The method of claim 19, wherein the step of assaying comprises measuring a level of the label in the mammalian cells.

39. The method of claim 38, wherein a level of the label that is less than or equal to a pre-determined threshold or a measured control value indicates that neutralizing antibodies against a SARS-CoV-2 S protein are present in the sample.

40. The method of claim 38, wherein a level of the label that is equal to or greater than a pre-determined threshold or a measured control value indicates that neutralizing antibodies against a SARS-CoV-2 S protein are not present in the sample.

41. The pseudotyped lentiviral vector particle according to claim 1, further comprising a heterologous polynucleotide that encodes a recombinase.


SEQUENCE LISTING (14 sequences)

SEQ ID NO: 1 - protein, 1273 aa, Artificial Sequence
  “SARS-CoV-2 S Protein”
  SARS-CoV-2 S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3819 bp, Artificial Sequence
  “nucleotide sequence encoding SARS-CoV-2 S Protein”
  nucleotide sequence encoding SARS-CoV-2 S Protein
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacc

SEQ ID NO: 3 - protein, 805 aa, Artificial Sequence [sequence omitted]

SEQ ID NO: 4 - DNA, 2415 bp, Artificial Sequence
  “nucleotide sequence encoding human ACE2 Protein”
  nucleotide sequence encoding human ACE2 Protein
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttt

SEQ ID NO: 5 - DNA, 2436 bp, Artificial Sequence
  “Nucleotide sequence of BamHI-XhoI DNA insert (hACE2)”
  Nucleotide sequence of BamHI-XhoI DNA insert (hACE2)
ggatccgccaccatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttaactcgag

SEQ ID NO: 6 - DNA, 1673 bp, Artificial Sequence
  “Nucleotide sequence of BamHI-XhoI DNA insert (Luciferase FireFly)”
  Nucleotide sequence of BamHI-XhoI DNA insert (Luciferase FireFly)
ggatccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgctcgag

SEQ ID NO: 7 - DNA, 617 bp, Artificial Sequence
  “Nucleotide sequence of XhoI-KpnI DNA insert (WPREm)”
  Nucleotide sequence of XhoI-KpnI DNA insert (WPREm)
ctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacc

SEQ ID NO: 8 - DNA, 3858 bp, Artificial Sequence
  “Nucleotide sequence of BamHI-XhoI DNA insert (Spike S High)”
  Nucleotide sequence of BamHI-XhoI DNA insert (Spike S High)
ggatcccgtacggccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataacgagcgcgcctcgag

SEQ ID NO: 9 - DNA, 795 bp, Artificial Sequence
  “Nucleotide sequence of Kanamycin”
  Nucleotide sequence of Kanamycin
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 10 - protein, 171 aa, Artificial Sequence
  “Protein sequence of Nanoluc”
  Protein sequence of Nanoluc
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 11 - DNA, 590 bp, Artificial Sequence
  “Nucleotide sequence of Nanolox”
  Nucleotide sequence of Nanolox
ataacttcgtatagcatacattatacgaagttatgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatattgaagcatatcgtatgtaatatgcttcaata

SEQ ID NO: 12 - DNA, 34 bp, Artificial Sequence
  “5′ LOXP sequence”
  5′ LOXP sequence
ataacttcgtatagcatacattatacgaagttat

SEQ ID NO: 13 - DNA, 34 bp, Artificial Sequence
  “3′ LOXP sequence”
  3′ LOXP sequence
tattgaagcatatcgtatgtaatatgcttcaata

SEQ ID NO: 14 - protein, 343 aa, Artificial Sequence
  “Protein sequence of recombinase cre Phage P1”
  Protein sequence of recombinase cre Phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD